(19) United States
(12) Patent Application Publication
(10) Pub. No.: US 2009/0104119 A1
(43) Pub. Date: Apr. 23, 2009

(54) Dendrimer Based Compositions And Methods Of Using The Same

(76) Inventors: Istvan J. Majoros, Ypsilanti, MI (US); Thommey P. Thomas, Dexter, MI (US); James R. Baker, Ann Arbor, MI (US); Zhengyi Cao, Ypsilanti, MI (US); Jolanta F. Kukowska-Latallo, Ann Arbor, MI (US)

Correspondence Address: Casimir Jones, S.C., 440 Science Drive, Suite 203, Madison, WI, 53711, US

(21) Appl. No.: 11/661,465

(22) PCT Filed: Aug. 25, 2005
(86) PCT No.: PCT/US2005/030278

§ 371 Date: Jun. 18, 2008

Related U.S. Application Data
(60) Provisional application No. 60/604,321, filed on Aug. 25, 2004.
(60) Provisional application No. 60/690,652, filed on Jun. 15, 2005.

(51) Int. Cl. A61K 47/48; C08G 69/26; A61P 35/00; A61K 31/519; A61K 51/06; A61K 38/00; A61K 49/00; A61K 31/166; A61K 31/675; A61K 31/4375; A61K 31/704; A61K 31/407; A61K 31/7048; A61K 31/138; A61K 31/337; A61K 31/505; A61K 31/7088; A61K 39/395; A61K 39/385

(52) U.S. Cl. 424/1.85; 528/353; 525/418; 525/54.1; 525/54.2; 514/249; 424/1.81; 514/2; 424/9.6; 514/615; 514/110; 514/283; 514/34; 514/411; 514/27; 514/648; 514/449; 514/274; 514/44; 424/178.1; 424/184.1

(57) ABSTRACT

The present invention relates to novel therapeutic and diagnostic dendrimers. In particular, the present invention is directed to dendrimer based multifunctional compositions and systems for use in disease diagnosis and therapy (e.g., cancer diagnosis and therapy). The compositions and systems comprise one or more components for targeting, imaging, sensing, and/or providing a therapeutic or diagnostic material and monitoring the response to therapy of a cell or tissue (e.g., a tumor).

The present invention claims priority to U.S. Provisional Patent Application Ser. Nos. 60/604,321, filed Aug. 25, 2004, and 60/690,652, filed Jun. 15, 2005, the disclosures of which are herein incorporated by reference in their entireties.

This invention was funded, in part, under NIH Contract NO1-CO-97111 and NCI Contract NO₁—CM-97065-32. The government may have certain rights in the invention.

### FIELD OF THE INVENTION

The present invention relates to novel therapeutic and diagnostic dendrimers. In particular, the present invention is directed to dendrimer based multifunctional compositions and systems for use in disease diagnosis and therapy (e.g., cancer diagnosis and therapy). The compositions and systems comprise one or more components for targeting, imaging, sensing, and/or providing a therapeutic or diagnostic material and monitoring the response to therapy of a cell or tissue (e.g., a tumor).

### BACKGROUND OF THE INVENTION

Cancer is the second leading cause of death, resulting in one out of every four deaths, in the United States. In 1997, the estimated total number of new diagnoses for lung, breast, prostate, colorectal and ovarian cancer was approximately two million. Due to the ever increasing aging population in the United States, it is reasonable to expect that rates of cancer incidence will continue to grow.

Cancer is currently treated using a variety of modalities including surgery, radiation therapy and chemotherapy. The choice of treatment modality will depend upon the type, location and dissemination of the cancer. For example, many common neoplasms, such as colon cancer, respond poorly to available therapies.

For tumor types that are responsive to current methods, only a fraction of cancers respond well to the therapies. In addition, despite the improvements in therapy for many cancers, most currently used therapeutic agents have severe side effects. These side effects often limit the usefulness of chemotherapeutic agents and result in a significant portion of cancer patients without any therapeutic options. Other types of therapeutic initiatives, such as gene therapy or immunotherapy, may prove to be more specific and have fewer side effects than chemotherapy. However, while showing some progress in a few clinical trials, the practical use of these approaches remains limited at this time.

Despite the limited success of existing therapies, the understanding of the underlying biology of neoplastic cells has advanced. The cellular events involved in neoplastic transformation and altered cell growth are now identified and the multiple steps in carcinogenesis of several human tumors have been documented (See e.g., Isaacs, Cancer 70:1810 (1992)). Oncogenes that cause unregulated cell growth have been identified and characterized as to genetic origin and function. Specific pathways that regulate the cell replication cycle have been characterized in detail and the proteins involved in this regulation have been cloned and characterized. Also, molecules that mediate apoptosis and negatively regulate cell growth have been clarified in detail (Kerr et al., Cancer 73:2013 (1994)). It has now been demonstrated that manipulation of these cell regulatory pathways has been able to stop growth and induce apoptosis in neoplastic cells (See e.g., Cohen and Tohoku, Exp. Med., 168:351 (1992) and Fujiwara et al., J. Natl. Cancer Inst., 86:458 (1994)). The metabolic pathways that control cell growth and replication in neoplastic cells are important therapeutic targets.

Despite these impressive accomplishments, many obstacles still exist before these therapies can be used to treat cancer cells in vivo. For example, these therapies require the identification of specific pathophysiologic changes in an individual's particular tumor cells. This requires mechanical invasion (biopsy) of a tumor and diagnosis typically by in vitro cell culture and testing. The tumor phenotype then has to be analyzed before a therapy can be selected and implemented. Such steps are time consuming, complex, and expensive.

There is a need for treatment methods that are selective for tumor cells compared to normal cells. Current therapies are only relatively specific for tumor cells. Although tumor targeting addresses this selectivity issue, it is not adequate, as most tumors do not have unique antigens. Further, the therapy ideally should have several, different mechanisms of action that work in parallel to prevent the selection of resistant neoplasms, and should be releasable by the physician after verification of the location and type of tumor. Finally, the therapy ideally should allow the physician to identify residual or minimal disease before and immediately after treatment, and to monitor the response to therapy. This is crucial since a few remaining cells may result in re-growth, or worse, lead to a tumor that is resistant to therapy. Identifying residual disease at the end of therapy (i.e., rather than after tumor regrowth) would facilitate eradication of the few remaining tumor cells.

Thus, an ideal therapy should have the ability to target a tumor, image the extent of the tumor (e.g., tumor metastasis) and identify the presence of the therapeutic agent in the tumor cells. Thus, therapies are needed that allows the physician to select therapeutic molecules based on the pathophysiologic abnormalities in the tumor cells, to activate the therapeutic agents in abnormal cells, to document the response to the therapy, and to identify residual disease.

### SUMMARY OF THE INVENTION

The present invention relates to novel therapeutic and diagnostic dendrimers. In particular, the present invention is directed to dendrimer based multifunctional compositions and systems for use in disease diagnosis and therapy (e.g., cancer diagnosis and therapy). The compositions and systems comprise one or more components for targeting, imaging, sensing, and/or providing a therapeutic or diagnostic material and monitoring the response to therapy of a cell or tissue (e.g., a tumor).

Accordingly, in some embodiments, the present invention provides a composition comprising a dendrimer, the dendrimer comprising a partially acetylated generation 5 (G5) dendrimer (e.g., polyamideamine (PAMAM), polypropylamine (POPAM), or PAMAM-POPAM dendrimer), the dendrimer further comprising one or more functional groups. The present invention is not limited to the use of G5 dendrimers. In some embodiments, the one or more functional groups comprise a therapeutic agent, a targeting agent, and/or an imaging agent. In some embodiments, at least one of the functional groups is conjugated to the dendrimers via an ester bond. In preferred embodiments, the therapeutic agent comprises a chemotherapeutic compound (e.g., methotrexate). In some preferred embodiments, the chemotherapeutic compound is conjugated to the dendrimer via an ester bond. In some preferred embodiments, the targeting agent comprises folic acid. In still other preferred embodiments, the imaging agent comprises fluorescein isothiocyanate or other detectable label. In some embodiments, the functional groups are one of a therapeutic agent, a targeting agent, an imaging agent, or a biological monitoring agent. In some embodiments, the G5 dendrimers are conjugated to the functional groups. In some embodiments, the conjugation comprises covalent bonds, ionic bonds, metallic bonds, hydrogen bonds, Van der Waals bonds, ester bonds or amide bonds.

In some embodiments of the present invention, the therapeutic agent comprises, but is not limited to, a chemotherapeutic agent, an anti-oncogenic agent, an anti-vascularizing agent, a tumor suppressor agent, an anti-microbial agent, or an expression construct comprising a nucleic acid encoding a therapeutic protein, although the present invention is not limited by the nature of the therapeutic agent. In further embodiments, the therapeutic agent is protected with a protecting group selected from photo-labile, radio-labile, and enzyme-labile protecting groups. In some embodiments, the chemotherapeutic agent is selected from a group consisting of, but not limited to, platinum complex, verapamil, podophylltoxin, carboplatin, procarbazine, mechloroethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, bleomycin, etoposide, tamoxifen, paclitaxel, taxol, transplatinum, 5-fluorouracil, vincristin, vinblastin, and methotrexate. In some embodiments, the anti-oncogenic agent comprises an antisense nucleic acid (e.g., RNA, molecule). In certain embodiments, the antisense nucleic acid comprises a sequence complementary to an RNA of an oncogene. In preferred embodiments, the oncogene includes, but is not limited to, abl, Bcl-2, Bcl-xL, erb, fms, gsp, hst, jun, myc, neu, raf; ras, ret, src, or trk. In some embodiments, the nucleic acid encoding a therapeutic protein encodes a factor including, but not limited to, a tumor suppressor, cytokine, receptor, inducer of apoptosis, or differentiating agent. In preferred embodiments, the tumor suppressor includes, but is not limited to, BRCA1, BRCA2, C-CAM, p16, p21, p53, p73, Rb, and p27. In preferred embodiments, the cytokine includes, but is not limited to, GMCSF, IL-1, IL-2, IL-3, IL-4, IL-5, IL-6, IL-7, IL-8, IL-9, IL-10, IL-11, IL-12, IL-13, IL-14, IL-15, β-interferon, γ-interferon, and TNF. In preferred embodiments, the receptor includes, but is not limited to, CFTR, EGFR, estrogen receptor, IL-2 receptor, and VEGFR. In preferred embodiments, the inducer of apoptosis includes, but is not limited to, AdE1B, Bad, Bak, Bax, Bid, Bik, Bim, Harakid, and ICE-CED3 protease. In some embodiments, the therapeutic agent comprises a short-half life radioisotope.

The present invention is not limited by type of anti-oncogenic agent or chemotherapeutic agent used (e.g., conjugated to a dendrimer of the present invention). Indeed, a variety of anti-oncogenic agents and chemotherapeutic agents are contemplated to be useful in the present invention including, but not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda).

Other anti-oncogenic agents and chemotherapeutic agents include antiproliferative agents (e.g., Piritrexim Isothionate), antiprostatic hypertrophy agents (e.g., Sitogluside), benign prostatic hyperplasia therapy agents (e.g., Tamsulosin Hydrochloride), prostate growth inhibitor agents (e.g., pentomone), and radioactive agents.

Yet other anti-oncogenic agents and chemotherapeutic agents may comprise anti-cancer supplementary potentiating agents, including tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); thiol depleters (e.g., buthionine and sulfoximine) and multiple drug resistance reducing agents such as Cremaphor EL.

Still other anti-oncogenic agents and chemotherapeutic agents are those selected from the group consisting of annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; hydroxyurea; docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′ deoxyadenosine; Fludarabine-PO.sub.4; mitoxantrone; mitozolomide; pentostatin; and tomudex.

Yet other anti-oncogenic agents and chemotherapeutic agents comprise taxanes (e.g., paclitaxel and docetaxel). In some embodiments, the anti-oncogenic agent or chemotherapeutic agent comprises tamoxifen or the aromatase inhibitor arimidex (e.g., anastrozole).

In some embodiments of the present invention, the biological monitoring agent comprises an agent that measures an effect of a therapeutic agent (e.g., directly or indirectly measures a cellular factor or reaction induced by a therapeutic agent), however, the present invention is not limited by the nature of the biological monitoring agent. In some embodiments, the monitoring agent is capable of measuring the amount of or detecting apoptosis caused by the therapeutic agent.

In some embodiments of the present invention, the imaging agent comprises a radioactive label including, but not limited to ¹⁴C, ³⁶Cl, ⁵⁷Co, ⁵⁸Co, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, Tc-99m, and ¹⁷⁵Yb. In some embodiments, the imaging agent comprises a fluorescing entity. In a preferred embodiment, the imaging agent is fluorescein isothiocyanate or 6-TAMARA.

In some embodiments of the present invention, the targeting agent includes, but is not limited to an antibody, receptor ligand, hormone, vitamin, and antigen, however, the present invention is not limited by the nature of the targeting agent. In some embodiments, the antibody is specific for a disease-specific antigen. In some preferred embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In a preferred embodiment, the receptor ligand is folic acid. Other embodiments that may be used with the present invention are described in U.S. Pat. No. 6,471,968 and WO 01/87348, each of which is herein incorporated by reference in their entireties.

In some embodiments, the dendrimers of the present invention (e.g., G5 PAMAM dendrimers) contain between 2-250, 10-200, or 100-150 reactive sites on the surface (See, e.g., Example 13). In preferred embodiments, the reactive sites comprise primary amine groups. In some embodiments, the dendrimers contain 50-250 reactive sites. In some embodiments, the dendrimers comprise 150-400 reactive sites. In preferred embodiments, the reactive sites are conjugated to functional groups comprising, but not limited to, therapeutic agents (e.g., methotrexate), targeting agents (e.g., folic acid), imaging agents (e.g., FITC) and biological monitoring agents.

In some embodiments, any one of the functional groups (e.g., therapeutic agent) is provided in multiple copies on a single dendrimer. Thus, in some embodiments, a single dendrimer comprises 2-100 copies of a single functional group (e.g., a therapeutic agent such as methotrexate). In some embodiments, a dendrimer comprises 2-5, 5-10, 10-20 or 20-50 copies of a single functional group. In some embodiments, a dendrimer comprises 5-20 copies. In some embodiments, a dendrimer comprises 50-100 or 100-200 copies of a functional group (e.g., a therapeutic agent, a targeting agent, or an imaging agent). In some embodiments, a dendrimer comprises greater than 200 copies of a functional group. The invention further provides a dendrimer that comprises multiple copies of two or more different functional group. For example, in some embodiments, the present invention provides a dendrimer that comprises multiple copies (e.g., 2-10, 5-10, 10-15, 15-50, 50-100, 100-200, or more than 200 copies) of one type of functional group (e.g., a therapeutic agent such as methotrexate or any one of the other targeting agents discussed herein) and multiple copies (e.g., 2-10, 15-50, 50-100, 100-200, or more than 200 copies) of a second type of functional group (e.g., a targeting agent such as folic acid or any one of the other targeting agents discussed herein). In some embodiments, a dendrimer comprises multiple copies of 2-10 different functional groups. For example, based on data generated during development of the present invention (e.g., data showing that a dendrimer may contain between 100-150 different locations (e.g., reactive sites such as primary amine groups), for conjugation of functional groups (See, e.g., Example 13), in some embodiments, a dendrimer may comprise 2-100 copies of a therapeutic agent (e.g., methotrexate), 2-100 copies of a targeting agent (e.g., folic acid) and 2-100 copies of an imaging agent (e.g., FITC or 6-TAMARA).

The present invention also provides methods for manufacturing dendrimers, the method comprising one or more of the steps (in any order): acetylating a G5 dendrimer to generate a partially acetylated dendrimer; conjugating an imaging agent (e.g., fluorescein isothiocyanate) to the partially acetylated dendrimer to generate a mono-functional dendrimer; conjugating a target agent (e.g., folic acid) to the partially acetylated mono-functional dendrimer to generate a two-functional dendrimer; conjugating glycidol to the partially acetylated two-functional dendrimer; and conjugating a therapeutic agent (e.g., methotrexate) to the partially acetylated two-functional glycidylated two-functional dendrimer. In preferred embodiments, the G5 dendrimer is generated according to the steps of: (a) obtaining an initiator core aliphatic diamine; (b) conducting a Michael reaction with a Michael acceptor; (c) condensating with a monoprotected diamine NH2-(CH2)ₙ-NHPG, (n=1-10); and (d) repeating steps (a)-(c); wherein the monoprotected diamine is used during the amide formation of each generation. The present invention is not limited by the nature of the initiator core aliphatic diamine chosen. In some embodiments, the initiator core aliphatic diamine is selected from the group comprising, but not limited to, NH2-(CH2)ₙ—NH2 (n=1-10), NH2-(CH2)ᵣ-NHPG, (n=1-10), NH2-((CH2)ₙNH2)₃ (n=1-10), or unsubstituted or substituted 1,2-; 1,3-; or 1,4-phenylenedi-n-alkylamine. In a preferred embodiment, the Michael acceptor is methyl acrylate. In some embodiments, the protecting group (PG) used is selected from the group comprising, but not limited to, t-butoxycarbamate (N-t-Boc), allyloxycarbamate (N-Alloc), benzylcarbamate (N-Cbz) 9-fluorenylmethylcarbamate (FMOC), or phthalimide (Phth).

The present invention also provides a composition comprising a dendrimer, the dendrimer comprising a protected core diamine. In some embodiments, the dendrimer comprises polyamideamine (PAMAM), polypropylamine (POPAM), or PAMAM-POPAM dendrimers. In particularly preferred embodiments, the core diamine is monoprotected. In some embodiments, the core diamine is NH2-(CH2)ₙ-NHPG (n=1-10). In preferred embodiments, the protected core diamine is NH2-CH2-CH2-NHPG. In some embodiments, the protected core diamine comprises a protecting group (PG), the protecting group selected from a group comprising, but not limited to, t-butoxycarbamate (N-t-Boc), allyloxycarbamate (N-Alloc), benzylcarbamate (N-Cbz), 9-fluorenylmethylcarbamate (FMOC), or phthalimide (Phth). In preferred embodiments, the dendrimer is partially acetylated. In particularly preferred embodiments, the dendrimer is conjugated to a functional group.

The present invention also provides a method of manufacturing a dendrimer comprising a protected core diamine, the method comprising the steps of: a) using a monoprotected initiator core aliphatic diamine NH2-(CH2)ₙ-NHPG, (n=1-10); b) conducting a Michael reaction with a Michael acceptor; c) condensating with equivalents of the monoprotected diamine; and d) repeating steps (a)-(c); wherein the monoprotected initiator core aliphatic diamine is used during the amide formation of each generation. In a preferred embodiment, the Michael acceptor is methyl acrylate. In some embodiments, each iteration produces a new generation (G=1-10) of a covalently bound radiating shell of repeating units with surface amino groups. In some embodiments, the protecting group (PG) comprises t-butoxycarbamate (N-t-Boc), allyloxycarbamate (N-Alloc), benzylcarbamate (N-Cbz) 9-fluorenylmethylcarbamate (FMOC), or phthalimide (Phth).

The present invention also provides a method of manufacturing a composition comprising a dendrimer, the method comprising the steps of a) using an initiator core aliphatic diamine; b) conducting a Michael reaction with a Michael acceptor; c) condensating with a monoprotected diamine NH2-(CH2)ₙ-NHPG, (n=1-10); and d) repeating steps (a)-(c); wherein the monoprotected diamine is used during the amide formation of each generation. The present invention is not limited by the nature of the initiator core aliphatic diamine chosen. In some embodiments, the initiator core aliphatic diamine is selected from NH2-(CH2)ₙ—NH2 (n=1-10), NH2-((CH2)ₙNH2)₃ (n=1-10), or unsubstituted or substituted 1,2-; 1,3-; or 1,4-phenylenedi-n-alkylamine. In some embodiments, the Michael acceptor is methyl acrylate. In some embodiments, each iteration produces a new generation (G=1-10) of a covalently bound radiating shell of repeating units with surface amino groups. In some embodiments, the protecting group (PG) comprises t-butoxycarbamate (N-t-Boc), allyloxycarbamate (N-Alloc), benzylcarbamate (N-Cbz) 9-fluorenylmethylcarbamate (FMOC), or phthalimide (Phth).

The present invention also provides a method of treating a disease (e.g., cancer or infectious disease) comprising administering to a subject suffering from or susceptible to the disease a therapeutically effective amount of a composition comprising a dendrimer of the present invention. In preferred embodiments, the dendrimers of the present invention are configured such that they are readily cleared from the subject (e.g., so that there is little to no detectable toxicity at efficacious doses). In some embodiments, the disease is a neoplastic disease, selected from, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma. In some embodiments, the disease is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome. In some embodiments, the disease is a viral disease selected from the group consisting of, but not limited to, viral disease caused by hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

The present invention also provides a method of treating a disease comprising administering to a subject suffering from or susceptible to the disease a therapeutically effective amount of a composition comprising a dendrimer, the dendrimer comprising a partially acetylated G5 PAMAM, POPAM, or PAMAM-POPAM dendrimer, the dendrimer further comprising one or more functional groups, the one or more functional groups selected from the group consisting of a therapeutic agent, a targeting agent, and an imaging agent. In some embodiments, the disease is a neoplastic disease. In some embodiments, the neoplastic disease is selected from the group consisting of leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

The present invention also provides a method of altering tumor growth in a subject, comprising providing a composition comprising a dendrimer, the dendrimer comprising a partially acetylated dendrimer, the dendrimer further comprising one or more functional groups, the one or more functional groups selected from the group consisting of a therapeutic agent, a targeting agent, and an imaging agent; and administering the composition to the subject under conditions such that the tumor growth is altered. In some embodiments, altering comprises inhibiting tumor growth in the subject. In some embodiments, altering comprises reducing the size of the tumor in the subject. In some embodiments, the composition comprising a dendrimer is co-administered with a chemotherapeutic agent or anti-oncogenic agent. In some embodiments, altering tumor growth sensitizes the tumor to chemotherapeutic or anti-oncogenic treatment.

### DESCRIPTION OF THE DRAWINGS

FIG. 1 depicts the (A) classical process, versus the (B) process in some embodiments of the present invention, used to synthesize PAPAM dendrimers.

FIG. 2 depicts a preferred protecting group (PG) of the protected core domain.

FIG. 3 depicts a core diamine when the core diamine is phenylenediamine, N—((CH2)ₙ—NH2)₃ (n=1-10).

FIG. 4 depicts the phenylenediamine of FIG. 3, but with substituents, where R and R1 are independently selected to be hydrogen, C1-C6 straight-chain or branched alkyls, C3-C6 cycloalkyls, C5-C10 aryl unsubstituted or substituted with C1-C6 alkyls, C1-C6 alkoxyls, 1,3-dioxolanyl, trihaloalkyl, carboxyl, C1-C6 dialkylamino, C1-C6 sulfanatoalkyl, C1-C6 sulfamylalkyl, or C1-C6 phosphanatoalkyl.

FIG. 5 depicts the synthesis of the phenylenediamines by catalytic reduction of the commercially available phenylenebisacetonitriles.

FIG. 6 depicts a synthetic scheme for generating multifunctional G5 PAMAM dendrimers.

FIG. 7 depicts potentiometric titration curves of G5 PAMAM dendrimers.

FIG. 8 depicts gel permeation chromatography eluograms of the partially acetylated carrier and final products, with the R1 signal and laser light scattering signal at 90° overlapping.

FIG. 9 depicts the theoretical and defected chemical structures of the G5 PAMAM dendrimer.

FIG. 10 depicts the (A) H1-NMR spectrum and (B) HPLC eluogram of the G5-Ac2 dendrimer.

FIG. 11 depicts the chemical structures of fluorescein isothiocyanate, folic acid and methotrexate, with the group used for conjugation marked with an asterisk.

FIG. 12 depicts the proton NMR imaging of fluorescein isothiocyanate, folic acid and methotrexate.

FIG. 13 depicts the HPLC eluogram of (A) G5-Ac²-FITC-OH-MTX^{e} and (B) G5-Ac³-FITC-OH-MTX^{e} at 305 nm.

FIG. 14 depicts the H1-NMR spectrum of G5-Ac²-FITC-FA-OH-MTX^{e}.

FIG. 15 depicts the HPLC eluogram of G5-Ac-FITC-FA-OH-MTX^{e} at 305 nm.

FIG. 16 depicts the UV spectra of free fluorescein isothiocyanate, folic acid and methotrexate.

FIG. 17 depicts the UV spectra of G5-Ac, G5-Ac³-FITC, G5-Ac³-FITC-FA, and G5-Ac³-FITC-FA-MTXe.

FIG. 18 depicts the (A) raw and (B) normalized fluorescence of dose-dependent binding of G5-FITC-FA-MTX in KB cells.

FIG. 19 depicts the effect of free FA on the uptake of the G5-FITC-FA and G5-FITC-FA-MTX in KB cells expressing high and low FA receptor.

FIG. 20 depicts confocal microscopy of KB cells treated with dendrimers.

FIG. 21 depicts (A) time course and (B) dose-dependent inhibition of cell growth using dendrimers.

FIG. 22 depicts growth inhibition of KB cells by dendrimers determined by XTT assays.

FIG. 23 depicts a comparison of cell growth inhibition using G5-FITC-FA-MTX and equimolar concentrations of mixtures of MTX and free FA.

FIG. 24 depicts reversal of G5-FA-MTX-induced inhibition of cell growth by free FA.

FIG. 25 depicts dendrimer stability in cell culture medium.

FIG. 26 depicts cytotoxicity of the dendrimers.

FIG. 27 shows the biodistribution of radiolabeled (A) nontargeted and (B) targeted conjugate in nu/nu mice bearing KB xenograft tumor depicted as a percentage of injected dose of dendrimer recovered per gram of organ.

FIG. 28 shows confocal microscopy analysis of cryosectioned tumor samples from SCID mice that were injected with 10 mmol of either (A) nontargeted G5-6-TAMRA or (B) targeted G5-FA-6-TAMRA conjugate (B) 15 hours or (D) 4 days before tumor isolation. Specific uptake by tumor cells of G5-FA-6-TAMRA versus G5-6-TAMRA is shown in (C).

FIG. 29 depicts tumor growth in SCID mice bearing KB xenografts during treatment with G5-FI-FA-MTX conjugate and free methotrexate (MTX).

FIG. 30 depicts survival rate of SCID mice bearing KB tumors.

FIG. 31 depicts a synthesis scheme for G5-Ac-AF-RGD.

FIG. 32 shows binding of G5-Ac-AF-RGD to HUVEC cells.

FIG. 33 shows binding of G5-Ac-AF-RGD to various cell lines.

FIG. 34 shows the dose dependent binding of G5-Ac-AF-RGD to HUVEC cells determined by confocal microscopy.

FIG. 35 shows the inhibition of uptake of G5-Ac-AF-RGD by HUVEC cells with addition of free peptide.

### DEFINITIONS

To facilitate an understanding of the present invention, a number of terms and phrases are defined below:

As used herein, the term “agent” refers to a composition that possesses a biologically relevant activity or property. Biologically relevant activities are activities associated with biological reactions or events or that allow the detection, monitoring, or characterization of biological reactions or events. Biologically relevant activities include, but are not limited to, therapeutic activities (e.g., the ability to improve biological health or prevent the continued degeneration associated with an undesired biological condition), targeting activities (e.g., the ability to bind or associate with a biological molecule or complex), monitoring activities (e.g., the ability to monitor the progress of a biological event or to monitor changes in a biological composition), imaging activities (e.g., the ability to observe or otherwise detect biological compositions or reactions), and signature identifying activities (e.g., the ability to recognize certain cellular compositions or conditions and produce a detectable response indicative of the presence of the composition or condition). The agents of the present invention are not limited to these particular illustrative examples. Indeed any useful agent may be used including agents that deliver or destroy biological materials, cosmetic agents, and the like. In preferred embodiments of the present invention, the agent or agents are associated with at least one dendrimer (e.g., incorporated into the dendrimer, surface exposed on the dendrimer, etc.). In some embodiments of the present invention, one dendrimer is associated with two or more agents that are different than” each other (e.g., one dendrimer associated with a targeting agent and a therapeutic agent). “Different than” refers to agents that are distinct from one another in chemical makeup and/or functionality.

As used herein, the term “nanodevice” refers to small (e.g., invisible to the unaided human eye) compositions containing or associated with one or more “agents.” In its simplest form, the nanodevice consists of a physical composition (e.g., a dendrimer) associated with at least one agent that provides biological functionality (e.g., a therapeutic agent). However, the nanodevice may comprise additional components (e.g., additional dendrimers and/or agents). In preferred embodiments of the present invention, the physical composition of the nanodevice comprises at least one dendrimer and a biological functionality is provided by at least one agent associated with a dendrimer.

The term “biologically active,” as used herein, refers to a protein or other biologically active molecules (e.g., catalytic RNA or small molecule) having structural, regulatory, or biochemical functions of a naturally occurring molecule.

The term “agonist,” as used herein, refers to a molecule which, when interacting with a biologically active molecule, causes a change (e.g., enhancement) in the biologically active molecule, which modulates the activity of the biologically active molecule. Agonists may include proteins, nucleic acids, carbohydrates, or any other molecules which bind or interact with biologically active molecules. For example, agonists can alter the activity of gene transcription by interacting with RNA polymerase directly or through a transcription factor.

The terms “antagonist” or “inhibitor,” as used herein, refer to a molecule which, when interacting with a biologically active molecule, blocks or modulates the biological activity of the biologically active molecule. Antagonists and inhibitors may include proteins, nucleic acids, carbohydrates, or any other molecules that bind or interact with biologically active molecules. Inhibitors and antagonists can effect the biology of entire cells, organs, or organisms (e.g., an inhibitor that slows tumor growth).

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “antigenic determinant” as used herein refers to that portion of an antigen that makes contact with a particular antibody (e.g., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (e.g., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (e.g., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labelled “A” and the antibody will reduce the amount of labelled A bound to the antibody.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “gene transfer system” refers to any means of delivering a composition comprising a nucleic acid sequence to a cell or tissue. For example, gene transfer systems include, but are not limited to vectors (e.g., retroviral, adenoviral, adeno-associated viral, and other nucleic acid-based delivery systems), microinjection of naked nucleic acid, and polymer-based delivery systems (e.g., liposome-based and metallic particle-based systems). As used herein, the term “viral gene transfer system” refers to gene transfer systems comprising viral elements (e.g., intact viruses and modified viruses) to facilitate delivery of the sample to a desired cell or tissue. As used herein, the term “adenovirus gene transfer system” refers to gene transfer systems comprising intact or altered viruses belonging to the family Adenoviridae.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables).

As used herein, the terms “photosensitizer,” and “photodynamic dye,” refer to materials which undergo transformation to an excited state upon exposure to a light quantum. Examples of photosensitizers and photodynamic dyes include, but are not limited to, Photofrin 2, benzoporphyrin, m-tetrahydroxyphenylchlorin, tin etiopurpurin, copper benzochlorin, and other porphyrins.

### DETAILED DESCRIPTION OF THE INVENTION

The present invention provides novel systems and compositions for the treatment, analysis, and monitoring of diseases (e.g., cancer). For example, the present invention provides systems and compositions that target, image, and sense pathophysiological defects, provide the appropriate therapeutic based on the diseased state, monitor the response to the delivered therapeutic, and identify residual disease. In preferred embodiments of the present invention, the compositions are small enough to readily enter a patient's or subjects cells and to be cleared from the body with little to no toxicity at therapeutic doses.

In preferred embodiments, the systems and compositions of the present invention are used in treatment and/or monitoring during cancer therapy. However, the systems and compositions of the present invention find use in the treatment and monitoring of a variety of disease states or other physiological conditions, and the present invention is not limited to use with any particular disease state or condition. Other disease states that find particular use with the present invention include, but are not limited to, cardiovascular disease, viral disease, inflammatory disease, and other proliferative disorders.

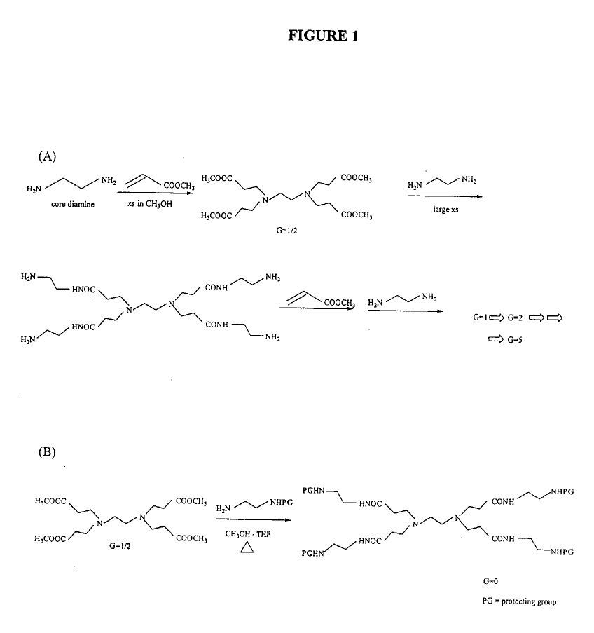

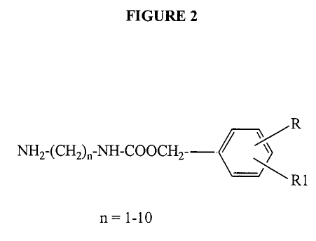

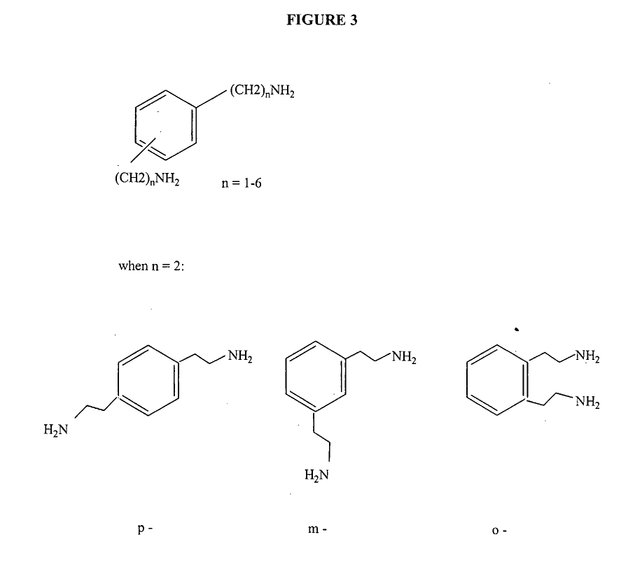

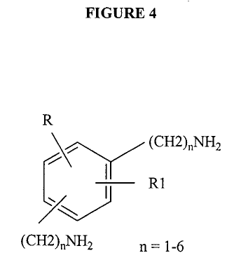

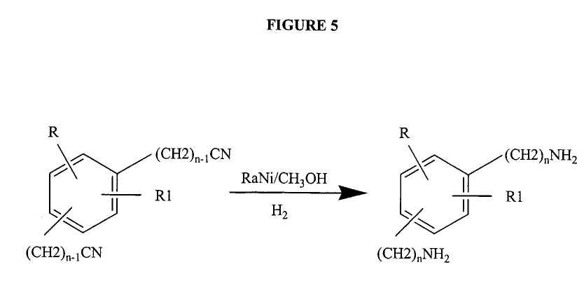

In preferred embodiments, the present invention provides a partially acetylated generation 5 (G5) polyamideamine (PAMAM), dendrimer (See, e.g., Example 1). In other preferred embodiments, the present invention provides methods of manufacturing a multifunctional G5 dendrimer (See, e.g., Example 2) and a method of manufacturing a dendrimer comprising a protected core diamine (See, e.g., FIGS. 1-5).

Preferred embodiments of the present invention provide compositions comprising a dendrimer conjugated to one or more functional groups, the functional groups including, but not limited to, therapeutic agents, biological monitoring components, biological imaging components, targeting components, and components to identify the specific signature of cellular abnormalities. As such, the therapeutic nanodevice is made up of individual dendrimers, each with one or more functional groups being specifically conjugated with or covalently linked to the dendrimer (See, e.g., Examples 2 and 6). In preferred embodiment, at least one of the functional groups is conjugated to the dendrimer via an ester bond (See, e.g., Example 7).

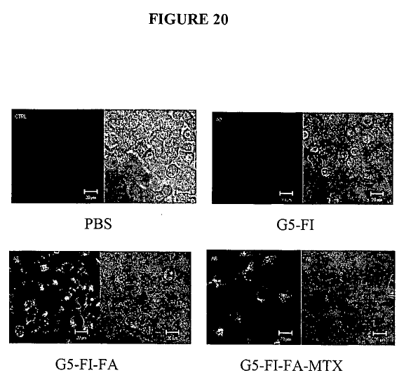

The following discussion describes individual component parts of the dendrimer and methods of making and using the same in some embodiments of the present invention. To illustrate the design and use of the systems and compositions of the present invention, the discussion focuses on specific embodiments of the use of the compositions in the treatment and monitoring of breast adenocarcinoma and colon adenocarcinoma. These specific embodiments are intended only to illustrate certain preferred embodiments of the present invention and are not intended to limit the scope thereof (e.g., compositions and methods of the present invention find use in the identification and treatment of prostate cancer and virally infected cells and tissue). In some embodiments, the dendrimers of the present invention target neoplastic cells through cell-surface moieties and are taken up by the tumor cell for example through receptor mediated endocytosis (See, e.g., Example 9, FIG. 20). In preferred embodiments, an imaging component (e.g., conjugated to a dendrimer of the present invention) allows the tumor to be imaged (e.g., through the use of MRI).

In some embodiments, the release of a therapeutic agent is facilitated by the therapeutic component being attached to a labile protecting group, such as, for example, cisplatin being attached to a photolabile protecting group that becomes released by laser light directed at those cells emitting the color of fluorescence activated as mentioned above (e.g., red-emitting cells). Optionally, the therapeutic device (e.g., compositions comprising dendrimers of the present invention) also may have a component to monitor the response of a target cell or tissue (e.g., a tumor) to therapy. For example, where a chemotherapeutic agent (e.g., methotrexate) conjugated to a dendrimer of the present invention induces apoptosis of a targeted cell, the caspase activity of the targeted cells may be used to activate a green fluorescence. This allows apoptotic cells to turn orange, (combination of red and green) while residual cells remain red. Any normal cells that are induced to undergo apoptosis in collateral damage fluoresce green.

As is clear from the above example, the use of the compositions of the present invention facilitates non-intrusive sensing, signaling, and intervention for cancer and other diseases and conditions. Since specific protocols of molecular alterations in cancer cells are identified using this technique, non-intrusive sensing through the dendrimers is achieved and may then be employed automatically against various tumor phenotypes.

### I. Dendrimers

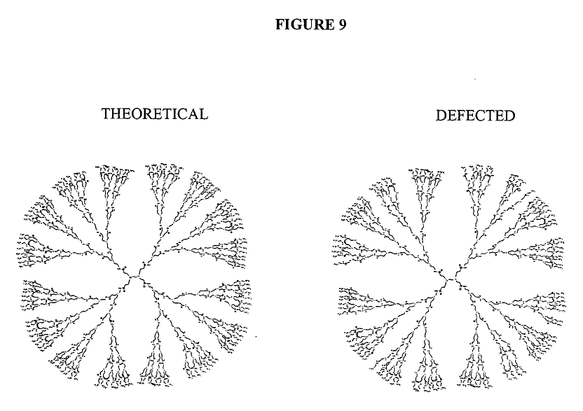

In preferred embodiments, the compositions of the present invention comprise dendrimers (See, e.g, FIGS. 1-5 and Example 2). Dendrimeric polymers have been described extensively (See, Tomalia, Advanced Materials 6:529 (1994); Angew, Chem. Int. Ed. Engl., 29:138 (1990); incorporated herein by reference in their entireties). Dendrimer polymers can be synthesized as defined spherical structures typically ranging from 1 to 20 nanometers in diameter. Methods for manufacturing a G5 PAMAM dendrimer with a protected core is shown (FIGS. 1-5). In some embodiments, the protected core diamine is NH2-CH2-CH2-NHPG. Molecular weight and the number of terminal groups increase exponentially as a function of generation (the number of layers) of the polymer (See, e.g., FIG. 9). Different types of dendrimers can be synthesized based on the core structure that initiates the polymerization process (See e.g., FIGS. 1-5).

The dendrimer core structures dictate several characteristics of the molecule such as the overall shape, density and surface functionality (Tomalia et al., Chem. Int. Ed. Engl., 29:5305 (1990)). Spherical dendrimers may have ammonia as a trivalent initiator core or ethylenediamine (EDA) as a tetravalent initiator core (See, e.g., FIG. 9). Recently described rod-shaped dendrimers (Yin et al., J. Am. Chem. Soc., 120:2678 (1998)) use polyethyleneimine linear cores of varying lengths (e.g., the longer the core, the longer the rod). Dendritic macromolecules are available commercially in kilogram quantities and are produced under current good manufacturing processes (GMP) for biotechnology applications.

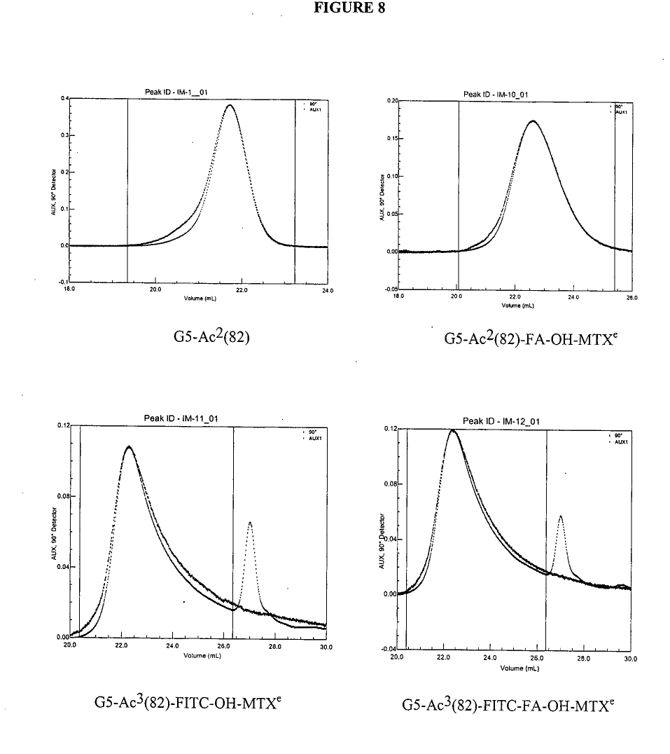

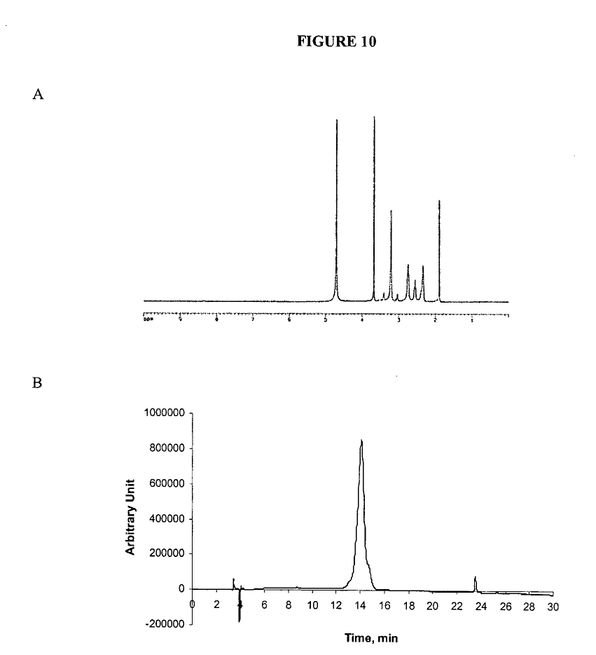

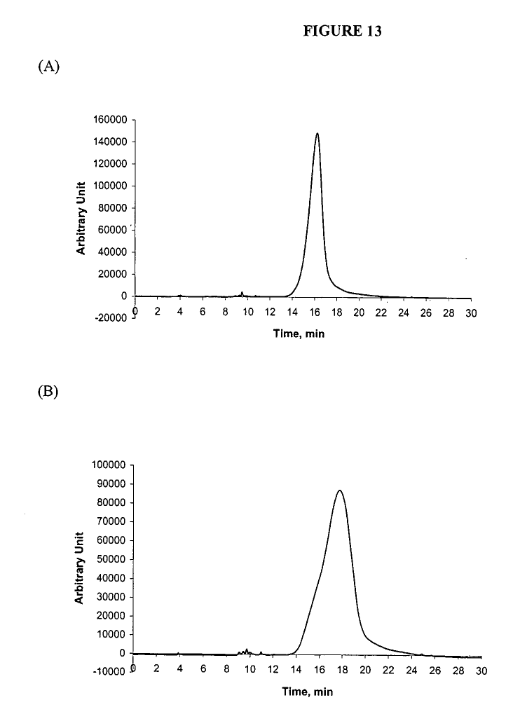

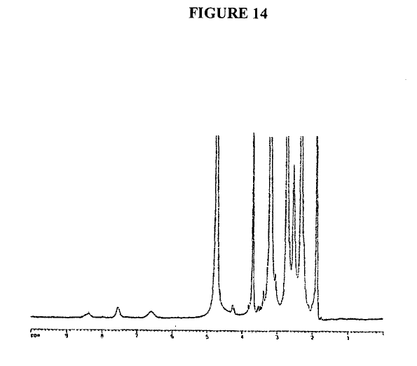

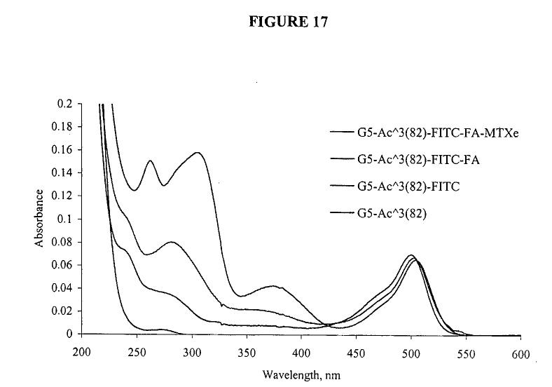

Dendrimers may be characterized by a number of techniques including, but not limited to, electrospray-ionization mass spectroscopy, ¹³C nuclear magnetic resonance spectroscopy, ¹H nuclear magnetic resonance spectroscopy (See, e.g., Example 5, FIG. 10(A) and Example 7, FIG. 14), high performance liquid chromatography (See, e.g., Example 5, FIG. 10(B); and Example 6, FIG. 13), size exclusion chromatography with multi-angle laser light scattering (See, e.g., Example 4, FIG. 8), ultraviolet spectrophotometry (See, e.g., Example 8, FIG. 17), capillary electrophoresis and gel electrophoresis. These tests assure the uniformity of the polymer population and are important for monitoring quality control of dendrimer manufacture for GMP applications and in vivo usage.

Numerous U.S. patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describe methods of making dense star polymers with terminal densities greater than conventional star polymers. These polymers have greater/more uniform reactivity than conventional star polymers, i.e. 3rd generation dense star polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

U.S. Pat. No. 6,471,968 describes a dendrimer complex comprising covalently linked first and second dendrimers, with the first dendrimer comprising a first agent and the second dendrimer comprising a second agent, wherein the first dendrimer is different from the second dendrimer, and where the first agent is different than the second agent.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

The use of dendrimers as metal ion carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjutants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polycation non-covalently coupled to the polynucleotide.

Dendrimer-antibody conjugates for use in in vitro diagnostic applications has previously been demonstrated (Singh et al., Clin. Chem., 40:1845 (1994)), for the production of dendrimer-chelant-antibody constructs, and for the development of boronated dendrimer-antibody conjugates (for neutron capture therapy); each of these latter compounds may be used as a cancer therapeutic (Wu et al., Bioorg. Med. Chem. Lett., 4:449 (1994); Wiener et al., Magn. Reson. Med. 31:1 (1994); Barth et al., Bioconjugate Chem. 5:58 (1994); and Barth et al.).

Some of these conjugates have also been employed in the magnetic resonance imaging of tumors (Wu et al., (1994) and Wiener et al., (1994), supra). Results from this work have documented that, when administered in vivo, antibodies can direct dendrimer-associated therapeutic agents to antigen-bearing tumors. Dendrimers also have been shown to specifically enter cells and carry either chemotherapeutic agents or genetic therapeutics. In particular, studies show that cisplatin encapsulated in dendrimer polymers has increased efficacy and is less toxic than cisplatin delivered by other means (Duncan and Malik, Control Rel. Bioact. Mater. 23:105 (1996)).

Dendrimers have also been conjugated to fluorochromes or molecular beacons and shown to enter cells. They can then be detected within the cell in a manner compatible with sensing apparatus for evaluation of physiologic changes within cells (Baker et al., Anal. Chem. 69:990 (1997)). Finally, dendrimers have been constructed as differentiated block copolymers where the outer portions of the molecule may be digested with either enzyme or light-induced catalysis (Urdea and Hom, Science 261:534 (1993)). This would allow the controlled degradation of the polymer to release therapeutics at the disease site and could provide a mechanism for an external trigger to release the therapeutic agents.

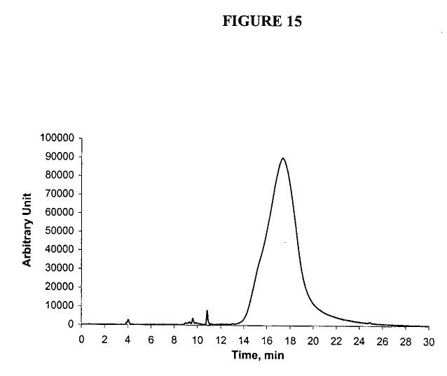

In some embodiments, the present invention provides dendrimers wherein one or more functional groups, each with a specific functionality, are provided in a single dendrimer (See, e.g., Examples 7 and 8, FIGS. 14 and 15). For example, a preferred composition of the present invention comprises a partially acetylated generation 5 (G5) PAMAM dendrimer further comprising a therapeutic agent, a targeting agent, and an imaging agent, wherein the therapeutic agent comprises methotrexate, the targeting agent comprises folic acid, and the imaging agent comprises fluorescein isothiocyanate (See, e.g., Examples 7 and 8). Hence, the present invention provides a single, multifunction dendrimer. In some embodiments, any one of the above functional groups (e.g., therapeutic agents) is provided in multiple copies on a single dendrimer. For example, in some embodiments, a single dendrimer comprises 2-100 copies of a single functional group (e.g., a therapeutic agent such as methotrexate). In yet other preferred embodiments, the present invention provides a partially acetylated generation 5 (G5) PAMAM dendrimer further comprising a therapeutic agent, a targeting agent, and an imaging agent, wherein the targeting agent comprises an RGD peptide (See, e.g., Example 14). In some embodiments, the present invention provides a a partially acetylated generation 5 (G5) PAMAM dendrimer comprising a therapeutic agent, a targeting agent, and an imaging agent, wherein the therapeutic agent comprises tritium (See, e.g., Example 13).

### II. Therapeutic Agents

A wide range of therapeutic agents find use with the present invention. Accordingly, the present invention is not limited by the type of therapeutic agent(s) that may be conjugated to a dendrimer of the present invention. Any therapeutic agent that can be associated with a dendrimer may be delivered using the methods, systems, and compositions of the present invention. To illustrate delivery of therapeutic agents, the following discussion focuses mainly on the delivery of methotrexate, cisplatin and taxol for the treatment of cancer. Also discussed are various photodynamic therapy compounds, and various antimicrobial compounds.

i. Methotrexate, Cisplatin and Taxol

The cytotoxicity of methotrexate depends on the duration for which a threshold intracellular level is maintained (Levasseur et al., Cancer Res 58, 5749 (1998); Goldman & Matherly, Pharmacol Ther 28, 77 (1985)). Cells contain high concentrations of DHFR, and, to shut off the DHFR activity completely, anti-folate levels six orders of magnitude higher than the Ki for DHFR is required (Sierrra & Goldman, Seminars in Oncology 26, 11 (1999)). Furthermore, less than 5% of the enzyme activity is sufficient for full cellular enzymatic function (White & Goldman, Biol Chem 256, 5722 (1981)). Cisplatin and Taxol have a well-defined action of inducing apoptosis in tumor cells (See e.g., Lanni et al., Proc. Natl. Acad. Sci., 94:9679 (1997); Tortora et al., Cancer Research 57:5107 (1997); and Zaffaroni et al., Brit. J. Cancer 77:1378 (1998)). However, treatment with these and other chemotherapeutic agents is difficult to accomplish without incurring significant toxicity. The agents currently in use are generally poorly water soluble, quite toxic, and given at doses that affect normal cells as wells as diseased cells. For example, paclitaxel (Taxol), one of the most promising anticancer compounds discovered, is poorly soluble in water.

Paclitaxel has shown excellent antitumor activity in a wide variety of tumor models such as the B16 melanoma, L1210 leukemias, MX-1 mammary tumors, and CS-1 colon tumor xenografts. However, the poor aqueous solubility of paclitaxel presents a problem for human administration. Accordingly, currently used paclitaxel formulations require a cremaphor to solubilize the drug. The human clinical dose range is 200-500 mg. This dose is dissolved in a 1:1 solution of ethanol:cremaphor and diluted to one liter of fluid given intravenously. The cremaphor currently used is polyethoxylated castor oil. It is given by infusion by dissolving in the cremaphor mixture and diluting with large volumes of an aqueous vehicle. Direct administration (e.g., subcutaneous) results in local toxicity and low levels of activity. Thus, there is a need for more efficient and effective delivery systems for these chemotherapeutic agents.

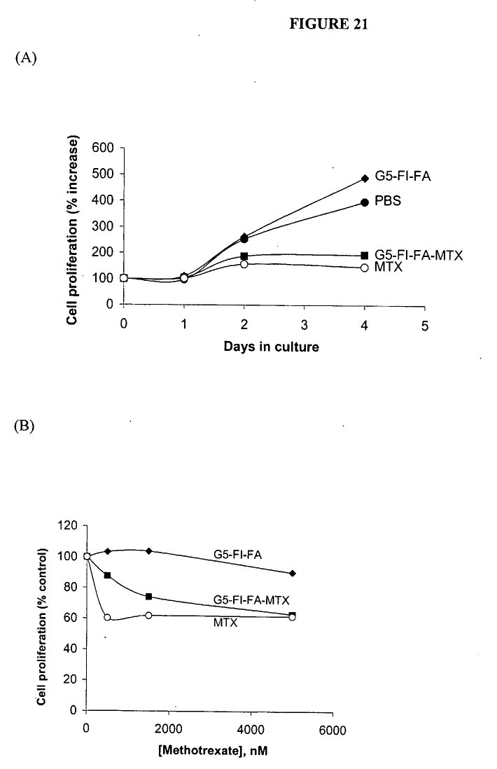

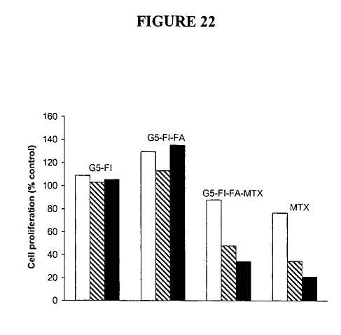

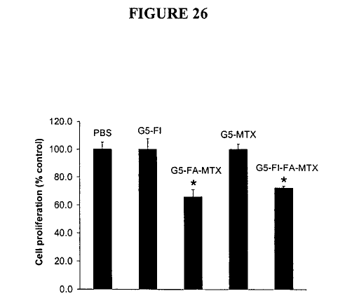

The present invention overcomes these problems by providing methods and compositions for specific drug delivery. The present invention also provides the ability to administer combinations of agents (e.g., two or more different therapeutic agents) to produce an additive effect. The use of multiple agents may be used to counter disease resistance to any single agent. For example, resistance of some cancers to single drugs (taxol) has been reported (Yu et al., Molecular Cell. 2:581 (1998)). Experiments conducted during the development of the present invention have demonstrated that methotrexate, conjugated to dendrimers, is able to efficiently kill cancer cells (See, Example 10, FIGS. 21 and 22, and Example 12, FIG. 26).

The present invention also provides the opportunity to monitor therapeutic success following delivery of methotrexate and/or cisplatin and/or Taxol to a subject. For example, measuring the ability of these drugs to induce apoptosis in vitro is reported to be a marker for in vivo efficacy (Gibb, Gynecologic Oncology 65:13 (1997)). Therefore, in addition to the targeted delivery of either one, two or all of these drugs (or other therapeutic agents) to provide effective anti-tumor therapy and reduce toxicity, the effectiveness of the therapy can be gauged by techniques of the present invention that monitor the induction of apoptosis. Importantly, these therapeutics are active against a wide-range of tumor types including, but not limited to, breast cancer and colon cancer (Akutsu et al., Eur. J. Cancer 31A:2341 (1995)).

Although the above discussion describes three specific agents, any agent (e.g., pharmaceutical) that is routinely used in a cancer therapy context finds use in the present invention. In treating cancer according to the invention, the therapeutic component of the dendrimer may comprise compounds including, but not limited to, adriamycin, 5-fluorouracil, etoposide, camptothecin, actinomycin-D, mitomycin C, or more preferably, cisplatin. The agent may be prepared and used as a combined therapeutic composition, or kit, by combining it with an immunotherapeutic agent, as described herein.

In some embodiments of the present invention, the dendrimer is contemplated to comprise one or more agents that directly cross-link nucleic acids (e.g., DNA) to facilitate DNA damage leading to a synergistic, antineoplastic agents of the present invention. Agents such as cisplatin, and other DNA alkylating agents may be used. Cisplatin has been widely used to treat cancer, with efficacious doses used in clinical applications of 20 mg/M² for 5 days every three weeks for a total of three courses. The dendrimers may be delivered via any suitable method, including, but not limited to, injection intravenously, subcutaneously, intratumorally, intraperitoneally, or topically (e.g., to mucosal surfaces).

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 Mg/M² at 21 day intervals for adriamycin, to 35-50 Mg/M² for etoposide intravenously or double the intravenous dose orally.

Agents that disrupt the synthesis and fidelity of nucleic acid precursors and subunits also lead to DNA damage and find use as chemotherapeutic agents in the present invention. A number of nucleic acid precursors have been developed. Particularly useful are agents that have undergone extensive testing and are readily available. As such, agents such as 5-fluorouracil (5-FU) are preferentially used by neoplastic tissue, making this agent particularly useful for targeting to neoplastic cells. The doses delivered may range from 3 to 15 mg/kg/day, although other doses may vary considerably according to various factors including stage of disease, amenability of the cells to the therapy, amount of resistance to the agents and the like.

The anti-cancer therapeutic agents that find use in the present invention are those that are amenable to incorporation into dendrimer structures or are otherwise associated with dendrimer structures such that they can be delivered into a subject, tissue, or cell without loss of fidelity of its anticancer effect. For a more detailed description of cancer therapeutic agents such as a platinum complex, veraparnil, podophyllotoxin, carboplatin, procarbazine, mechlorethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate and other similar anti-cancer agents, those of skill in the art are referred to any number of instructive manuals including, but not limited to, the Physician's Desk reference and to Goodman and Gilman's “Pharmaceutical Basis of Therapeutics” ninth edition, Eds. Hardman et al., 1996.

In some embodiments, the drugs are preferably attached to the dendrimers with photocleavable linkers. For example, several heterobifunctional, photocleavable linkers that find use with the present invention are described by Ottl et al. (Ottl et al., Bioconjugate Chem., 9:143 (1998)). These linkers can be either water or organic soluble. They contain an activated ester that can react with amines or alcohols and an epoxide that can react with a thiol group. In between the two groups is a 3,4-dimethoxy-6-nitrophenyl photoisomerization group, which, when exposed to near-ultraviolet light (365 nm), releases the amine or alcohol in intact form. Thus, the therapeutic agent, when linked to the compositions of the present invention using such linkers, may be released in biologically active or activatable form through exposure of the target area to near-ultraviolet light.

In a preferred embodiment, methotrexate is conjugated to the dendrimer via an ester bond (See, e.g., Example 7). In an exemplary embodiment, the alcohol group of taxol is reacted with the activated ester of the organic-soluble linker. This product in turn is reacted with the partially-thiolated surface of appropriate dendrimers (the primary amines of the dendrimers can be partially converted to thiol-containing groups by reaction with a sub-stoichiometric amount of 2-iminothiolano). In the case of cisplatin, the amino groups of the drug are reacted with the water-soluble form of the linker. If the amino groups are not reactive enough, a primary amino-containing active analog of cisplatin, such as Pt(II) sulfadiazine dichloride (Pasani et al., Inorg. Chim. Acta 80:99 (1983) and Abel et al, Eur. J. Cancer 9:4 (1973)) can be used. Thus conjugated, the drug is inactive and will not harm normal cells. When the conjugate is localized within tumor cells, it is exposed to laser light of the appropriate near-UV wavelength, causing the active drug to be released into the cell.

Similarly, in other embodiments of the present invention, the amino groups of cisplatin (or an analog thereof) is linked with a very hydrophobic photocleavable protecting group, such as the 2-nitrobenzyloxycarbonyl group (Pillai, V. N. R. Synthesis: 1-26 (1980)). With this hydrophobic group attached, the drug is loaded into and very preferentially retained by the hydrophobic cavities within the PAMAM dendrimer (See e.g., Esfand et al., Pharm. Sci., 2:157 (1996)), insulated from the aqueous environment. When exposed to near-LV light (about 365 nm), the hydrophobic group is cleaved, leaving the intact drug. Since the drug itself is hydrophilic, it diffuses out of the dendrimer and into the tumor cell, where it initiates apoptosis.

An alternative to photocleavable linkers are enzyme cleavable linkers. A number of photocleavable linkers have been demonstrated as effective anti-tumor conjugates and can be prepared by attaching cancer therapeutics, such as doxorubicin, to water-soluble polymers with appropriate short peptide linkers (See e.g., Vasey et al., Clin. Cancer Res., 5:83 (1999)). The linkers are stable outside of the cell, but are cleaved by thiolproteases once within the cell. In a preferred embodiment, the conjugate PKI is used. As an alternative to the photocleavable linker strategy, enzyme-degradable linkers, such as Gly-Phe-Leu-Gly may be used.

The present invention is not limited by the nature of the therapeutic technique. For example, other conjugates that find use with the present invention include, but are not limited to, using conjugated boron dusters for BNCT (Capala et al., Bioconjugate Chem., 7:7 (1996)), the use of radioisotopes, and conjugation of toxins such as ricin to the nanodevice.

ii. Photodynamic Therapy

Photodynamic therapeutic agents may also be used as therapeutic agents in the present invention. In some embodiments, the dendrimeric compositions of the present invention containing photodynamic compounds are illuminated, resulting in the production of singlet oxygen and free radicals that diffuse out of the fiberless radiative effector to act on the biological target (e.g., tumor cells or bacterial cells). Some preferred photodynamic compounds include, but are not limited to, those that can participate in a type II photochemical reaction:

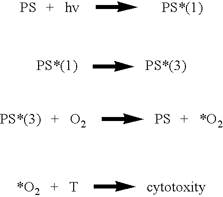

where PS=photosenstizer, PS*(1)=excited singlet state of PS, PS*(3)=excited triplet state of PS, hv=light quantum, *O₂=excited singlet state of oxygen, and T=biological target. Other photodynamic compounds useful in the present invention include those that cause cytotoxicity by a different mechanism than singlet oxygen production (e.g., copper benzochlorin, Selman, et al., Photochem. Photobiol., 57:681-85 (1993), incorporated herein by reference). Examples of photodynamic compounds that find use in the present invention include, but are not limited to Photofrin 2, phtalocyanins (See e.g., Brasseur et al., Photochem. Photobiol., 47:705-11 (1988)), benzoporphyrin, tetrahydroxyphenylporphyrins, naphtalocyanines (See e.g., Firey and Rodgers, Photochem. Photobiol., 45:535-38 (1987)), sapphyrins (Sessler et al., Proc. SPIE, 1426.318-29 (1991)), porphinones (Chang et al., Proc. SPEE, 1203:281-86 (1990)), tin etiopurpurin, ether substituted porphyrins (Pandey et al., Photochem. Photobiol., 53:65-72 (1991)), and cationic dyes such as the phenoxazines (See e.g., Cincotta et al., SPIE Proc., 1203:202-10 (1990)).
iii. Antimicrobial Therapeutic Agents

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins, antisense nucleic acids, membrane disruptive agents and the like, used alone or in combination. Indeed, any type of antibiotic may be used including, but not limited to, anti-bacterial agents, anti-viral agents, anti-fungal agents, and the like.

### III. Signature Identifying Agents

In certain embodiments, the nanodevices of the present invention contain one or more signature identifying agents that are activated by, or are able to interact with, a signature component (“signature”). In preferred embodiments, the signature identifying agent is an antibody, preferably a monoclonal antibody, that specifically binds the signature (e.g., cell surface molecule specific to a cell to be targeted).

In some embodiments of the present invention, tumor cells are identified. Tumor cells have a wide variety of signatures, including the defined expression of cancer-specific antigens such as Muc1, HER-2 and mutated p53 in breast cancer. These act as specific signatures for the cancer, being present in 30% (HER-2) to 70% (mutated p53) of breast cancers. In a preferred embodiment, a dendrimer of the present invention comprises a monoclonal antibody that specifically binds to a mutated version of p53 that is present in breast cancer.

In some embodiments of the present invention, cancer cells expressing susceptibility genes are identified. For example, in some embodiments, there are two breast cancer susceptibility genes that are used as specific signatures for breast cancer: BRCA1 on chromosome 17 and BRCA2 on chromosome 13. When an individual carries a mutation in either BRCA1 or BRCA2, they are at an increased risk of being diagnosed with breast or ovarian cancer at some point in their lives. These genes participate in repairing radiation-induced breaks in double-stranded DNA. It is thought that mutations in BRCA1 or BRCA2 might disable this mechanism, leading to more errors in DNA replication and ultimately to cancerous growth.

In addition, the expression of a number of different cell surface receptors find use as targets for the binding and uptake of the nano-device. Such receptors include, but are not limited to, EGF receptor, folate receptor, FGR receptor 2, and the like.

In some embodiments of the present invention, changes in gene expression associated with chromosomal abborations are the signature component. For example, Burkitt lymphoma results from chromosome translocations that involve the Myc gene. A chromosome translocation means that a chromosome is broken, which allows it to associate with parts of other chromosomes. The classic chromosome translocation in Burkitt lymphoma involves chromosome 8, the site of the Myc gene. This changes the pattern of Myc expression, thereby disrupting its usual function in controlling cell growth and proliferation.

In other embodiments, gene expression associated with colon cancer are identified as the signature component. Two key genes are known to be involved in colon cancer: MSH2 on chromosome 2 and MLH1 on chromosome 3. Normally, the protein products of these genes help to repair mistakes made in DNA replication. If the MSH2 and MLH1 proteins are mutated, the mistakes in replication remain unrepaired, leading to damaged DNA and colon cancer. MEN1 gene, involved in multiple endocrine neoplasia, has been known for several years to be found on chromosome 11, was more finely mapped in 1997, and serves as a signature for such cancers. In preferred embodiments of the present invention, an antibody specific for the altered protein or for the expressed gene to be detected is complexed with nanodevices of the present invention.

In yet another embodiment, adenocarcinoma of the colon has defined expression of CEA and mutated p53, both well-documented tumor signatures. The mutations of p53 in some of these cell lines are similar to that observed in some of the breast cancer cells and allows for the sharing of a p53 sensing component between the two nanodevices for each of these cancers (i.e., in assembling the nanodevice, dendrimers comprising the same signature identifying agent may be used for each cancer type). Both colon and breast cancer cells may be reliably studied using cell lines to produce tumors in nude mice, allowing for optimization and characterization in animals.

From the discussion above it is clear that there are many different tumor signatures that find use with the present invention, some of which are specific to a particular type of cancer and others which are promiscuous in their origin. The present invention is not limited to any particular tumor signature or any other disease-specific signature. For example, tumor suppressors that find use as signatures in the present invention include, but are not limited to, p53, Muc1, CEA, p16, p21, p27, CCAM, RB, APC, DCC, NF-1, NF-2, WT-1, MEN-1, MEN-II, p73, VHL, FCC and MCC.

### IV. Biological Imaging Component

In some embodiments of the present invention, the nanodevice comprises at least one dendrimer-based nanoscopic building block that can be readily imaged. The present invention is not limited by the nature of the imaging component used. In some embodiments of the present invention, imaging modules comprise surface modifications of quantum dots (See e.g., Chan and Nie, Science 281:2016 (1998)) such as zinc sulfide-capped cadmium selenide coupled to biomolecules (Sooklal, Adv. Mater., 10:1083 (1998)).

However, in preferred embodiments, the imaging module comprises dendrimers produced according to the “nanocomposite” concept (Balogh et al., Proc. of ACS PMSE 77:118 (1997) and Balogh and Tomalia, J. Am. Che. Soc., 120:7355 (1998)). In these embodiments, dendrimers are produced by reactive encapsulation, where a reactant is preorganized by the dendrimer template and is then subsequently immobilized in/on the polymer molecule by a second reactant. Size, shape, size distribution and surface functionality of these nanoparticles are determined and controlled by the dendritic macromolecules. These materials have the solubility and compatibility of the host and have the optical or physiological properties of the guest molecule (i.e., the molecule that permits imaging). While the dendrimer host may vary according to the medium, it is possible to load the dendrimer hosts with different compounds and at various guest concentration levels. Complexes and composites may involve the use of a variety of metals or other inorganic materials. The high electron density of these materials considerably simplifies the imaging by electron microscopy and related scattering techniques. In addition, properties of inorganic atoms introduce new and measurable properties for imaging in either the presence or absence of interfering biological materials. In some embodiments of the present invention, encapsulation of gold, silver, cobalt, iron atoms/molecules and/or organic dye molecules such as fluorescein are encapsulated into dendrimers for use as nanoscopic composite labels/tracers, although any material that facilitates imaging or detection may be employed. In a preferred embodiment, the imaging agent is fluorescein isothiocyanate

In some embodiments of the present invention, imaging is based on the passive or active observation of local differences in density of selected physical properties of the investigated complex matter. These differences may be due to a different shape (e.g., mass density detected by atomic force microscopy), altered composition (e.g. radiopaques detected by X-ray), distinct light emission (e.g., fluorochromes detected by spectrophotometry), different diffraction (e.g., electron-beam detected by TEM), contrasted absorption (e.g., light detected by optical methods), or special radiation emission (e.g., isotope methods), etc. Thus, quality and sensitivity of imaging depend on the property observed and on the technique used. The imaging techniques for cancerous cells have to provide sufficient levels of sensitivity to is observe small, local concentrations of selected cells. The earliest identification of cancer signatures requires high selectivity (i.e., highly specific recognition provided by appropriate targeting) and the highest possible sensitivity.

### A. Magnetic Resonance Imaging

Once the targeted nanodevice has attached to (or been internalized into) tumor cells, one or more modules on the device serve to image its location. Dendrimers have already been employed as biomedical imaging agents, perhaps most notably for magnetic resonance imaging (MRI) contrast enhancement agents (See e.g., Wiener et al., Mag. Reson. Med. 31:1 (1994); an example using PAMAM dendrimers). These agents are typically constructed by conjugating chelated paramagnetic ions, such as Gd(III)-diethylenetriaminepentaacetic acid (Gd(III)-DTPA), to water-soluble dendrimers. Other paramagnetic ions that may be useful in this context of the include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof. In some embodiments of the present invention, the dendrimer is also conjugated to a targeting group, such as epidermal growth factor (EGF), to make the conjugate specifically bind to the desired cell type (e.g., in the case of EGF, EGFR-expressing tumor cells). In a preferred embodiment of the present invention, DTPA is attached to dendrimers via the isothiocyanate of DTPA as described by Wiener (Wiener et al., Mag. Reson. Med. 31:1 (1994)).

Dendrimeric MRI agents are particularly effective due to the polyvalency, size and architecture of dendrimers, which results in molecules with large proton relaxation enhancements, high molecular relaxivity, and a high effective concentration of paramagnetic ions at the target site. Dendrimeric gadolinium contrast agents have even been used to differentiate between benign and malignant breast tumors using dynamic MRI, based on how the vasculature for the latter type of tumor images more densely (Adam et al., Ivest. Rad. 31:26 (1996)). Thus, MRI provides a particularly useful imaging system of the present invention.

### B. Microscopic Imaging

Static structural microscopic imaging of cancerous cells and tissues has traditionally been performed outside of the patient. Classical histology of tissue biopsies provides a fine illustrative example, and has proven a powerful adjunct to cancer diagnosis and treatment. After removal, a specimen is sliced thin (e.g., less than 40 microns), stained, fixed, and examined by a pathologist. If images are obtained, they are most often 2-D transmission bright-field projection images. Specialized dyes are employed to provide selective contrast, which is almost absent from the unstained tissue, and to also provide for the identification of aberrant cellular constituents. Quantifying sub-cellular structural features by using computer-assisted analysis, such as in nuclear ploidy determination, is often confounded by the loss of histologic context owing to the thinness of the specimen and the overall lack of 3-D information. Despite the limitations of the static imaging approach, it has been invaluable to allow for the identification of neoplasia in biopsied tissue. Furthermore, its use is often the crucial factor in the decision to perform invasive and risky combinations of chemotherapy, surgical procedures, and radiation treatments, which are often accompanied by severe collateral tissue damage, complications, and even patient death.

The nanodevices of the present invention allow functional microscopic imaging of tumors and provide improved methods for imaging. The methods find use in vivo, in vitro, and ex vivo. For example, in one embodiment of the present invention, dendrimers of the present invention are designed to emit light or other detectable signals upon exposure to light. Although the labeled dendrimers may be physically smaller than the optical resolution limit of the microscopy technique, they become self-luminous objects when excited and are readily observable and measurable using optical techniques. In some embodiments of the present invention, sensing fluorescent biosensors in a microscope involves the use of tunable excitation and emission filters and multiwavelength sources (Farkas et al., SPEI 2678:200 (1997)). In embodiments where the imaging agents are present in deeper tissue, longer wavelengths in the Near-infrared (NMR) are used (See e.g., Lester et al., Cell Mol. Biol. 44:29 (1998)). Dendrimeric biosensing in the Near-IR has been demonstrated with dendrimeric biosensing antenna-like architectures (Shortreed et al., J. Phys. Chem., 101:6318 (1997)). Biosensors that find use with the present invention include, but are not limited to, fluorescent dyes and molecular beacons.

In some embodiments of the present invention, in vivo imaging is accomplished using functional imaging techniques. Functional imaging is a complementary and potentially more powerful techniques as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce. When excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments of the present invention, biosensor-comprising dendrimers of the present invention are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic dendrimeric biosensors for pH, oxygen concentration, Ca²+ concentration, and other physiologically relevant analytes.

### V. Biological Monitoring Component

The biological monitoring or sensing component of the nanodevice of the present invention is one which that can monitor the particular response in the tumor cell induced by an agent (e.g., a therapeutic agent provided by the therapeutic component of the nanodevice). While the present invention is not limited to any particular monitoring system, the invention is illustrated by methods and compositions for monitoring cancer treatments. In preferred embodiments of the present invention, the agent induces apoptosis in cells and monitoring involves the detection of apoptosis. In particular embodiments, the monitoring component is an agent that fluoresces at a particular wavelength when apoptosis occurs. For example, in a preferred embodiment, caspase activity activates green fluorescence in the monitoring component. Apoptotic cancer cells, which have turned red as a result of being targeted by a particular signature with a red label, turn orange while residual cancer cells remain red. Normal cells induced to undergo apoptosis (e.g., through collateral damage), if present, will fluoresce green.

In these embodiments, fluorescent groups such as fluorescein are employed in the monitoring component. Fluorescein is easily attached to the dendrimer surface via the isothiocyanate derivatives, available from Molecular Probes, Inc. This allows the nanodevices to be imaged with the cells via confocal microscopy. Sensing of the effectiveness of the nanodevices is preferably achieved by using fluorogenic peptide enzyme substrates. For example, apoptosis caused by the therapeutic agents results in the production of the peptidase caspase-1 (ICE). Calbiochem sells a number of peptide substrates for this enzyme that release a fluorescent moiety. A particularly useful peptide for use in the present invention is: MCA-Tyr-Glu-Val-Asp-Gly-Trp-Lys-(DNP)-NH₂ (SEQ ID NO: 1) where MCA is the (7-methoxycoumarin-4-yl)acetyl and DNP is the 2,4-dinitrophenyl group (Talanian et al., J. Biol. Chem., 272: 9677 (1997)). In this peptide, the MCA group has greatly attenuated fluorescence, due to fluorogenic resonance energy transfer (FRET) to the DNP group. When the enzyme cleaves the peptide between the aspartic acid and glycine residues, the MCA and DNP are separated, and the MCA group strongly fluoresces green (excitation maximum at 325 nm and emission maximum at 392 nm).

In preferred embodiments of the present invention, the lysine end of the peptide is linked to the nanodevice, so that the MCA group is released into the cytosol when it is cleaved. The lysine end of the peptide is a useful synthetic handle for conjugation because, for example, it can react with the activated ester group of a bifunctional linker such as Mal-PEG-OSu. Thus the appearance of green fluorescence in the target cells produced using these methods provides a clear indication that apoptosis has begun (if the cell already has a red color from the presence of aggregated quantum dots, the cell turns orange from the combined colors).

Additional fluorescent dyes that find use with the present invention include, but are not limited to, acridine orange, reported as sensitive to DNA changes in apoptotic cells (Abrams et al., Development 117:29 (1993)) and cis-parinaric acid, sensitive to the lipid peroxidation that accompanies apoptosis (Hockenbery et al., Cell 75:241 (1993)). It should be noted that the peptide and the fluorescent dyes are merely exemplary. It is contemplated that any peptide that effectively acts as a substrate for a caspase produced as a result of apoptosis finds use with the present invention.

### VI. Targeting Components

As described above, another component of the present invention is that the nanodevice compositions are able to specifically target a particular cell type (e.g., tumor cell). Although an understanding of the mechanism is not necessary to practice the present invention and the present invention is not limited to any particular mechanism of action, in some embodiments, the nanodevice targets a cell (e.g., a neoplastic cell) through a cell surface moiety and is taken into the cell through receptor mediated endocytosis.

Any moiety known to be located on the surface of target cells (e.g. tumor cells) finds use with the present invention. For example, an antibody directed against such a moiety targets the compositions of the present invention to cell surfaces containing the moiety. Alternatively, the targeting moiety may be a ligand directed to a receptor present on the cell surface or vice versa. In a preferred embodiment of the present invention, the targeting moiety is the folic acid receptor. In some embodiments, the targeting moiety is an RGD peptide receptor (e.g., αᵥβ₃ integrin). Similarly, vitamins also may be used to target the therapeutics of the present invention to a particular cell.

In some embodiments of the present invention, the targeting moiety may also function as a signatures component. For example, tumor specific antigens including, but not limited to, carcinoembryonic antigen, prostate specific antigen, tyrosinase, ras, a sialyl)-lewis antigen, erb, MAGE-1, MAGE-3, BAGE, MN, gp100, gp75, p97, proteinase 3, a mucin, CD81, CID9, CD63; CD53, CD38, CO-029, CA125, GD2, GM2 and O-acetyl GD3, M-TAA, M-fetal or M-urinary find use with the present invention. Alternatively the targeting moiety may be a tumor suppressor, a cytokine, a chemokine, a tumor specific receptor ligand, a receptor, an inducer of apoptosis, or a differentiating agent.

Tumor suppressor proteins contemplated for targeting include, but are not limited to, p16, p21, p27, p53, p73, Rb, Wilns tumor (WT-1), DCC, neurofibromatosis type I (NF-1), von Hippel-Lindau (VHL) disease tumor suppressor, Maspin, Brush-1, BRCA-1, BRCA-2, the multiple tumor suppressor (MTS), gp95/p97 antigen of human melanoma, renal cell carcinoma-associated G250 antigen, KS ¼ pan-carcinoma antigen, ovarian carcinoma antigen (CA125), prostate specific antigen, melanoma antigen gp75, CD9, CD63, CD53, CD37, R2, CD81, C0029, TI-1, L6 and SAS. Of course these are merely exemplary tumor suppressors and it is envisioned that the present invention may be used in conjunction with any other agent that is or becomes known to those of skill in the art as a tumor suppressor.

In preferred embodiments of the present invention targeting is directed to factors expressed by an oncogene. These include, but are not limited to, tyrosine kinases, both membrane-associated and cytoplasmic forms, such as members of the Src family, serine/threonine kinases, such as Mos, growth factor and receptors, such as platelet derived growth factor (PDDG), SMALL GTPases (G proteins) including the ras family, cyclin-dependent protein kinases (cdk), members of the myc family members including c-myc, N-myc, and L-myc and bcl-2 and family members.

Cytokines that may be targeted by the present invention include, but are not limited to, IL-1, IL-2, IL-3, IL-4, IL-5, IL-6, IL-7, IL-8, IL-9, IL-10, ILA 1, IL-12, IL-13, IL-14, IL-15, TNF, GMCSF, α-interferon and γ-interferon. Chemokines that may be used include, but are not limited to, M1P1α, M1P1β, and RANTES.

Enzymes that may be targeted by the present invention include, but are not limited to, cytosine deaminase, hypoxanthine-guanine phosphoribosyltransferase, galactose-1-phosphate uridyltransferase, phenylalanine hydroxylase, glucocerbrosidase, sphingomyelinase, .alpha.-L-iduronidase, glucose-6-phosphate dehydrogenase, HSV thymidine kinase, and human thymidine kinase.

Receptors and their related ligands that find use in the context of the present invention include, but are not limited to, the folate receptor, adrenergic receptor, growth hormone receptor, luteinizing hormone receptor, estrogen receptor, epidermal growth factor receptor, fibroblast growth factor receptor, and the like.

Hormones and their receptors that find use in the targeting aspect of the present invention include, but are not limited to, growth hormone, prolactin, placental lactogen, luteinizing hormone, follicle-stimulating hormone, chorionic gonadotropin, thyroid-stimulating hormone, leptin, adrenocorticotropin (ACTH), angiotensin I, angiotensin II, .beta.-endorphin, .beta.-melanocyte stimulating hormone (β-MSH), cholecystokinin, endothelin I, galanin, gastric inhibitory peptide (GIP), glucagon, insulin, amylin, lipotropins, GLP-1 (7-37) neurophysins, and somatostatin.

In addition, the present invention contemplates that vitamins (both fat soluble and non-fat soluble vitamins) placed in the targeting component of the nanodevice may be used to target cells that have receptors for, or otherwise take up these vitamins. Particularly preferred for this aspect are the fat soluble vitamins, such as vitamin D and its analogues, vitamin E, Vitamin A, and the like or water soluble vitamins such as Vitamin C, and the like.

In some embodiments of the present invention, any number of cancer cell targeting groups are attached to dendrimers. The targeting dendrimers are, in turn, conjugated to a core dendrimer. Thus the nanodevice of the present invention is such that it is specific for targeting cancer cells (i.e., much more likely to attach to cancer cells and not to healthy cells). In addition, the polyvalency of dendrimers allows the attachment of polyethylene glycol (PEG) or polyethyloxazoline (PEOX) chains to help increase the blood circulation time and decrease the immunogenicity of the conjugates.

In preferred embodiments of the present invention, targeting groups are conjugated to dendrimers with either short (e.g., direct coupling), medium (e.g. using small-molecule bifunctional linkers such as SPDP, sold by Pierce Chemical Company), or long (e.g., PEG bifunctional linkers, sold by Shearwater Polymers) linkages. Since dendrimers have surfaces with a large number of functional groups, more than one targeting group may be attached to each dendrimer. As a result, there are multiple binding events between the dendrimer and the target cell. In these embodiments, the dendrimers have a very high affinity for their target cells via this “cooperative binding” or polyvalent interaction effect.

For steric reasons, the smaller the ligands, the more can be attached to the surface of a dendrimer. Recently, Wiener reported that dendrimers with attached folic acid would specifically accumulate on the surface and within tumor cells expressing the high-affinity folate receptor (hFR) (Wiener et al., Invest. Radiol., 32:748 (1997)). The hFR receptor is expressed or upregulated on epithelial tumors, including breast cancers. Control cells lacking hFR showed no significant accumulation of folate-derivatized dendrimers. Folic acid can be attached to full generation PAMAM dendrimers via a carbodiimide coupling reaction. Folic acid is a good targeting candidate for the dendrimers, with its small size and a simple conjugation procedure.

A larger, yet still relatively small ligand is epidermal growth factor (EGF), a single-chain peptide with 53 amino acid residues. It has been shown that PAMAM dendrimers conjugated to EGF with the linker SPDP bind to the cell surface of human glioma cells and are endocytosed, accumulating in lysosomes (Casale et al., Bioconjugate Chem., 7:7 (1996)). Since EGF receptor density is up to 100 times greater on brain tumor cells compared to normal cells, EGF provides a useful targeting agent for these kinds of tumors. Since the EGF receptor is also overexpressed in breast and colon cancer, EGF may be used as a targeting agent for these cells as well. Similarly, the fibroblast growth factor receptors (EGER) also bind the relatively small polypeptides (FGF), and many are known to be expressed at high levels in breast tumor cell lines (particularly FGF1, 2 and 4) (Penault-Llorca et al., Int. J. Cancer 61:170 (1995)).

In preferred embodiments of the present invention, the targeting moiety is an antibody or antigen binding fragment of an antibody (e.g., Fab units). For example, a well-studied antigen found on the surface of many cancers (including breast HER2 tumors) is glycoprotein p185, which is exclusively expressed in malignant cells (Press et al., Oncogene 5:953 (1990)). Recombinant humanized anti-HER2 monoclonal antibodies (rhuMabHER2) have even been shown to inhibit the growth of HER2 overexpressing breast cancer cells, and are being evaluated (in conjunction with conventional chemotherapeutics) in phase III clinical trials for the treatment of advanced breast cancer (Pegram et al., Proc. Am. Soc. Clin. Oncol., 14:106 (1995)). Park and Papahadjopoulos have attached Fab fragments of rhuMabHER2 to small unilamellar liposomes, which then can be loaded with the chemotherapeutic doxorubicin (dox) and targeted to HER2 overexpressing tumor xenografts (Park et al., Cancer Lett., 118:153 (1997) and Kirpotin et al., Biochem., 36:66 (1997)). These dox-loaded “immunoliposomes” showed increased cytotoxicity against tumors compared to corresponding non-targeted dox-loaded liposomes or free dox, and decreased systemic toxicity compared to free dox.

Antibodies can be generated to allow for the targeting of antigens or immunogens (e.g., tumor, tissue or pathogen specific antigens) on various biological targets (e.g., pathogens, tumor cells, normal tissue). Such antibodies include, but are not limited to polyclonal, monoclonal, chimeric, single chain, Fab fragments, and an Fab expression library.

In some preferred embodiments, the antibodies recognize tumor specific epitopes (e.g., TAG-72 (Kjeldsen et al., Cancer Res. 48:2214-2220 (1988); U.S. Pat. Nos. 5,892,020; 5,892,019; and 5,512,443); human carcinoma antigen (U.S. Pat. Nos. 5,693,763; 5,545,530; and 5,808,005); TP1 and TP3 antigens from osteocarcinoma cells (U.S. Pat. No. 5,855,866); Thomsen-Friedenreich (TF) antigen from adenocarcinoma cells (U.S. Pat. No. 5,110,911); “KC-4 antigen” from human prostrate adenocarcinoma (U.S. Pat. Nos. 4,708,930 and 4,743,543); a human colorectal cancer antigen (U.S. Pat. No. 4,921,789); CA125 antigen from cystadenocarcinoma (U.S. Pat. No. 4,921,790); DF3 antigen from human breast carcinoma (U.S. Pat. Nos. 4,963,484 and 5,053,489); a human breast tumor antigen (U.S. Pat. No. 4,939,240); p97 antigen of human melanoma (U.S. Pat. No. 4,918,164); carcinoma or orosomucoid-related antigen (CORA) (U.S. Pat. No. 4,914,021); a human pulmonary carcinoma antigen that reacts with human squamous cell lung carcinoma but not with human small cell lung carcinoma (U.S. Pat. No. 4,892,935); T and Tn haptens in glycoproteins of human breast carcinoma (Springer et al., Carbohydr. Res. 178:271-292 (1988)), MSA breast carcinoma glycoprotein termed (Tjandra et al., Br. J. Surg. 75:811-817 (1988)); MFGM breast carcinoma antigen (Ishida et al., Tumor Biol. 10:12-22 (1989)); DU-PAN-2 pancreatic carcinoma antigen (Lan et al., Cancer Res. 45:305-310 (1985)); CA125 ovarian carcinoma antigen (Hanisch et al., Carbohydr. Res. 178:29-47 (1988)); YH206 lung carcinoma antigen (Hinoda et al., (1988) Cancer J. 42:653-658 (1988)). Each of the foregoing references are specifically incorporated herein by reference.

In other preferred embodiments, the antibodies recognize specific pathogens (e.g., Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Staphylococcus aureus, human papilloma virus, human immunodeficiency virus, rubella virus, polio virus, and the like).

Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Kohler and Milstein (Kohler and Milstein, Nature 256:495-497 (1975)), as well as the trioma technique, the human B-cell hybridoma technique (See e.g., Kozbor et al. Immunol. Today 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

In an additional embodiment of the invention, monoclonal antibodies can be produced in germ-free animals utilizing recent technology (See e.g., PCT/US90/02545). According to the invention, human antibodies may be used and can be obtained by using human hybridomas (Cote et al., Proc. Natl. Acad. Sci. U.S.A. 80:2026-2030 (1983)) or by transforming human B cells with EBV virus in vitro (Cole et al., in Monoclonal Antibodies and Cancer Therapy, Alan R. Liss, pp. 77-96 (1985)).

According to the invention, techniques described for the production of single chain antibodies (U.S. Pat. No. 4,946,778; herein incorporated by reference) can be adapted to produce specific single chain antibodies. An additional embodiment of the invention utilizes the techniques described for the construction of Fab expression libraries (Huse et al., Science 246:1275-1281 (1989)) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of the antibody molecule; the Fab′ fragments that can be generated by reducing the disulfide bridges of the F(ab′)2 fragment, and the Fab fragments that can be generated by treating the antibody molecule with papain and a reducing agent.

In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.).

The dendrimer systems of the present invention have many advantages over liposomes, such as their greater stability, better control of their size and polydispersity, and generally lower toxicity and immunogenicity (See e.g., Duncan et al, Polymer Preprints 39:180 (1998)). Thus, in some embodiments of the present invention, anti-HER2 antibody fragments, as well as other targeting antibodies are conjugated to dendrimers, as targeting agents for the nanodevices of the present invention.

In some embodiments, for cancer (e.g., breast cancer), the cell surface may be targeted with folic acid, EGF, FGF, and antibodies (or antibody fragments) to the tumor-associated antigens MUCI, cMet receptor and CD56 (NCAM). Once internalized into the cell, the nanodevice binds (via conjugated antibodies) to HER2, MUCI or mutated p53.

The bifunctional linkers SPDP and SMCC and the longer Mal-PEG-OSu linkers are particularly useful for antibody-dendrimer conjugation. In addition, many tumor cells contain surface lectins that bind to oligosaccharides, with specific recognition arising chiefly from the terminal carbohydrate residues of the latter (Sharon and L is, Science 246:227 (1989)). Attaching appropriate monosaccharides to nonglycosylated proteins such as BSA provides a conjugate that binds to tumor lectin much more tightly than the free monosaccharide (Monsigny et al., Biochemie 70:1633 (1988)).

Mannosylated PAMAM dendrimers bind mannoside-binding lectin up to 400 more avidly than monomeric mannosides (Page and Roy, Bioconjugate Chem., 8:714 (1997)). Sialylated dendrimers and other dendritic polymers bind to and inhibit a variety of sialate-binding viruses both in vitro and in vivo. By conjugating multiple monosaccharide residues (e.g., .alpha.-galactoside, for galactose-binding cells) to dendrimers, polyvalent conjugates are created with a high affinity for the corresponding type of tumor cell. The attachment reaction are easily carried out via reaction of the terminal amines with commercially-available .alpha.-galactosidyl-phenylisothiocyanate. The small size of the carbohydrates allows a high concentration to be present on the dendrimer surface.

A very flexible method to identify and select appropriate peptide targeting groups is the phage display technique (See e.g., Cortese et al., Curr. Opin. Biotechol., 6:73 (1995)), which can be conveniently carried out using commercially available kits. The phage display procedure produces a large and diverse combinatorial library of peptides attached to the surface of phage, which are screened against immobilized surface receptors for tight binding. After the tight-binding, viral constructs are isolated and sequenced to identify the peptide sequences. The cycle is repeated using the best peptides as starting points for the next peptide library. Eventually, suitably high-affinity peptides are identified and then screened for biocompatibility and target specificity. In this way, it is possible to produce peptides that can be conjugated to dendrimers, producing multivalent conjugates with high specificity and affinity for the target cell receptors (e.g., tumor cell receptors) or other desired targets.

Related to the targeting approaches described above is the “pretargeting” approach (See e.g., Goodwin and Meares, Cancer (suppl.) 80:2675 (1997)). An example of this strategy involves initial treatment of the patient with conjugates of tumor-specific monoclonal antibodies and streptavidin. Remaining soluble conjugate is removed from the bloodstream with an appropriate biotinylated clearing agent. When the tumor-localized conjugate is all that remains, a radiolabeled, biotinylated agent is introduced, which in turn localizes at the tumor sites by the strong and specific biotin-streptavidin interaction. Thus, the radioactive dose is maximized in dose proximity to the cancer cells and minimized in the rest of the body where it can harm healthy cells.

It has been shown that if streptavidin molecules bound to a polystyrene well are first treated with a biotinylated dendrimer, and then radiolabeled streptavidinis introduced, up to four of the labeled streptavidin molecules are bound per polystyrene-bound streptavidin (Wilbur et al., Bioconjugate Chem., 9:813 (1998)). Thus, biotinylated dendrimers may be used in the methods of the present invention, acting as a polyvalent receptor for the radiolabel in vivo, with a resulting amplification of the radioactive dosage per bound antibody conjugate. In the preferred embodiments of the present invention, one or more multiply-biotinylated module(s) on the clustered dendrimer presents a polyvalent target for radiolabeled or boronated (Barth et al., Cancer Investigation 14:534 (1996)) avidin or streptavidin, again resulting in an amplified dose of radiation for the tumor cells.

Dendrimers may also be used as clearing agents by, for example, partially biotinylating a dendrimer that has a polyvalent galactose or mannose surface. The conjugate-clearing agent complex would then have a very strong affinity for the corresponding hepatocyte receptors.

In other embodiments of the present invention, an enhanced permeability and retention (EPR) method is used in targeting. The enhanced permeability and retention (EPR) effect is a more “passive” way of targeting tumors (See, Duncan and Sat, Ann. Oncol., 9:39 (1998)). The EPR effect is the selective concentration of macromolecules and small particles in the tumor microenvironment, caused by the hyperpermeable vasculature and poor lymphatic drainage of tumors. The dendrimer compositions of the present invention provide ideal polymers for this application, in that they are relatively rigid, of narrow polydispersity, of controlled size and surface chemistry, and have interior “cargo” space that can carry and then release antitumor drugs. In fact, PAMAM dendrimer-platinates have been shown to accumulate in solid tumors (Pt levels about 50 times higher than those obtained with cisplatin) and have in vivo activity in solid tumor models for which cisplatin has no effect (Malik et al., Proc. Int'l. Symp. Control. Rel. Bioact. Mater., 24:107 (1997) and Duncan et al., Polymer Preprints 39:180 (1998)).

The targeting moieties of the present invention may recognize a variety of other epitopes on biological targets (e.g., on pathogens). In some embodiments, molecular recognition elements are incorporated to recognize, target or detect a variety of pathogenic organisms including, but not limited to, sialic acid to target HIV (Wies et al., Nature 333: 426 (1988)), influenza (White et al., Cell 56: 725 (1989)), Chlamydia (Infect. 1 mm. 57: 2378 (1989)), Neisseria meningitidis, Streptococcus suis, Salmonella, mumps, newcastle, and various viruses, including reovirus, Sendai virus, and myxovirus; and 9-OAC sialic acid to target coronavirus, encephalomyelitis virus, and rotavirus; non-sialic acid glycoproteins to detect cytomegalovirus (Virology 176: 337 (1990)) and measles virus (Virology 172: 386 (1989)); CD4 (Khatzman et al., Nature 312: 763 (1985)), vasoactive intestinal peptide (Sacerdote et al., J. of Neuroscience Research 18: 102 (1987)), and peptide T (Ruff et al., FEBS Letters 211: 17 (1987)) to target HIV; epidermal growth factor to target vaccimia (Epstein et al., Nature 318: 663 (1985)); acetylcholine receptor to target rabies (Lentz et al., Science 215: 182 (1982)); Cd3 complement receptor to target Epstein-Barr virus (Carel et al., J. Biol. Chem. 265: 12293 (1990)); .beta.-adrenergic receptor to target reovirus (Co et al., Proc. Natl. Acad. Sci. 82: 1494 (1985)); ICAM-1 (Marlin et al., Nature 344: 70 (1990)), N-CAM, and myelin-associated glycoprotein MAb (Shephey et al., Proc. Natl. Acad. Sci. 85: 7743 (1988)) to target rhinovirus; polio virus receptor to target polio virus (Mendelsohn et al., Cell 56: 855 (1989)); fibroblast growth factor receptor to target herpes virus (Kaner et al., Science 248: 1410 (1990)); oligomannose to target Escherichia coli; ganglioside G.sub.M1 to target Neissetia meningitidis; and antibodies to detect a broad variety of pathogens (e.g., Neissetia gonorrhoeae, V. vulnificus, V. parahaemolyticus, V. cholerae, and V. alginolyticus).

In some embodiments of the present invention, the targeting moities are preferably nucleic acids (e.g., RNA or DNA). In some embodiments, the nucleic acid targeting moities are designed to hybridize by base pairing to a particular nucleic acid (e.g., chromosomal DNA, mRNA, or ribosomal RNA). In other embodiments, the nucleic acids bind a ligand or biological target. Nucleic acids that bind the following proteins have been identified: reverse transcriptase, Rev and Tat proteins of HIV (Tuerk et al., Gene 137(1):33-9 (1993)); human nerve growth factor (Binkley et al., Nuc. Acids Res. 23(16):3198-205 (1995)); and vascular endothelial growth factor (Jellinek et al., Biochem. 83(34):10450-6 (1994)). Nucleic acids that bind ligands are preferably identified by the SELEX procedure (See e.g., U.S. Pat. Nos. 5,475,096; 5,270,163; and 5,475,096; and in PCT publications WO 97/38134, WO 98/33941, and WO 99/07724, all of which are herein incorporated by reference), although many methods are known in the art.

### VII. Synthesis and Conjugation

The present section provides a description of the synthesis and formation of the individual components (i.e., individual dendrimers containing one or more of the components described above) of the nanodevice and the conjugation of such components to the dendrimer.

In preferred embodiments of the present invention, the preparation of PAMAM dendrimers is performed according to a typical divergent (building up the macromolecule from an initiator core) synthesis. It involves a two-step growth sequence that consists of a Michael addition of amino groups to the double bond of methyl acrylate (MA) followed by the amidation of the resulting terminal carbomethoxy, —(CO₂ CH₃) group, with ethylenediamine (EDA).

In the first step of this process, ammonia is allowed to react under an inert nitrogen atmosphere with MA (molar ratio: 1:4.25) at 47° C. for 48 hours. The resulting compound is referred to as generation=0, the star-branched PAMAM tri-ester. The next step involves reacting the tri-ester with an excess of EDA to produce the star-branched PAMAM tri-amine (G=O). This reaction is performed under an inert atmosphere (nitrogen) in methanol and requires 48 hours at 0.degree. C. for completion. Reiteration of this Michael addition and amidation sequence produces generation=1.

Preparation of this tri-amine completes the first full cycle of the divergent synthesis of PAMAM dendrimers. Repetition of this reaction sequence results in the synthesis of larger generation (G=1-5) dendrimers (i.e., ester- and amine-terminated molecules, respectively). For example, the second iteration of this sequence produces generation 1, with an hexa-ester and hexa-amine surface, respectively. The same reactions are performed in the same way as for all subsequent generations from 1 to 9, building up layers of branch cells giving a core-shell architecture with precise molecular weights and numbers of terminal groups as shown above. Carboxylate-surfaced dendrimers can be produced by hydrolysis of ester-terminated PAMAM dendrimers, or reaction of succinic anhydride with amine-surfaced dendrimers (e.g., full generation PAMAM, POPAM or POPAM-PAMAM hybrid dendrimers).

Various dendrimers can be synthesized based on the core structure that initiates the polymerization process. These core structures dictate several important characteristics of the dendrimer molecule such as the overall shape, density, and surface functionality (Tomalia et al., Angew. Chem. Int. Ed. Engl., 29:5305 (1990)). S pherical dendrimers derived from ammonia possess trivalent initiator cores, whereas EDA is a tetra-valent initiator core. Recently, rod-shaped dendrimers have been reported which are based upon linear poly(ethyleneimine) cores of varying lengths the longer the core, the longer the rod (Yin et al., J. Am. Chem. Soc., 120:2678 (1998)).

In preferred embodiments, the dendrimer of the present invention comprises a protected core diamine. In particularly preferred embodiments, the protected initiator core diamine is NH2-(CH2)ₙ—NHPG, (n=1-10). In other preferred embodiments, the intitor core is selected from the group comprising, but not limited to, NH2-(CH2)ₙ-NH2 (n=1-10), NH2-((CH2)ₙNH2)₃ (n=1-10), or unsubstituted or substituted 1,2-; 1,3-; or 1,4-phenylenedi-n-alkylamine, with a monoprotected diamine (e.g., NH2-(CH2)ₙ-NHPG) used during the amide formation of each generation. In these approaches, the protected diamine allows for the large scale production of dendrimers without the production of non-uniform nanostructures that can make characterization and analysis difficult. By limiting the reactivity of the diamine to only one terminus, the opportunities of dimmer/polymer formation and intramolecular reactions are obviated without the need of employing large excesses of diamine. The terminus monoprotected intermediates can be readily purified since the protecting groups provide suitable handle for productive purifications by classical techniques like crystallization and or chromatography.

The protected intermediates can be deprotected in a deprotection step, and the resulting generation of the dendrimer subjected to the next iterative chemical reaction without the need for purification. The invention is not limited to a particular protecting group. Indeed a variety of protecting groups are contemplated including, but not limited to, t-butoxycarbamate (N-t-Boc), allyloxycarbamate (N-Alloc), benzylcarbamate (N-Cbz), 9-fluorenylmethylcarbamate (FMOC), or phthalimide (Phth). In preferred embodiments of the present invention, the protecting group is benzylcarbamate (N-Cbz). N-Cbz is ideal for the present invention since it alone can be easily cleaved under “neutral” conditions by catalytic hydrogenation (Pd/C) without resorting to strongly acidic or basic conditions needed to remove an F-MOC group. The use of protected monomers finds particular use in high through-put production runs because a lower amount of monomer can be used, reducing production costs.

The dendrimers may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC) size exclusion chromatography (SEC) (equipped with multi-angle laser light scattering, dual UV and refractive index detectors), capillary electrophoresis and get electrophoresis. These analytical methods assure the uniformity of the dendrimer population and are important in the quality control of dendrimer production for eventual use in in vivo applications. Most importantly, extensive work has been performed with dendrimers showing no evidence of toxicity when administered intravenously (Roberts et al., J. Biomed. Mater. Res., 30:53 (1996) and Bourne et al., J. Magnetic Resonance Imaging, 6:305 (1996)).

### VIII. Evaluation of Anti-Tumor Efficacy and Toxicity of Nanodevice

The anti-tumor effects of various therapeutic agents on cancer cell lines and primary cell cultures may be evaluated using the nanodevices of the present invention. For example, in preferred embodiments, assays are conducted, in vitro, using established tumor cell line models or primary culture cells (See, e.g., Examples 10-12), or alternatively, assays can be conducted in vivo using animal models (See, e.g., Example 13).

### A. Quantifying the Induction of Apoptosis of Human Tumor Cells In Vitro

In an exemplary embodiment of the present invention, the nanodevices of the present invention are used to assay apoptosis of human tumor cells in vitro. Testing for apoptosis in the cells determines the efficacy of the therapeutic agent. Multiple aspects of apoptosis can and should be measured. These aspects include those described above, as well as aspects including, but are not limited to, measurement of phosphatidylserine (PS) translocation from the inner to outer surface of plasma membrane, measurement of DNA fragmentation, detection of apoptosis related proteins, and measurement of Caspase-3 activity.

### B. In Vitro Toxicology

In some embodiments of the present invention, to gain a general perspective into the safety of a particular nanodevice platform or component of that system, toxicity testing is performed. Toxicological information may be derived from numerous sources including, but not limited to, historical databases, in vitro testing, and in vivo animal studies.

In vitro toxicological methods have gained popularity in recent years due to increasing desires for alternatives to animal experimentation and an increased perception to the potential ethical, commercial, and scientific value. In vitro toxicity testing systems have numerous advantages including improved efficiency, reduced cost, and reduced variability between experiments. These systems also reduce animal usage, eliminate confounding systemic effects (e.g., immunity), and control environmental conditions.

Although any in vitro testing system may be used with the present invention, the most common approach utilized for in vitro examination is the use of cultured cell models. These systems include freshly isolated cells, primary cells, or transformed cell cultures. Cell culture as the primary means of studying in vitro toxicology is advantageous due to rapid screening of multiple cultures, usefulness in identifying and assessing toxic effects at the cellular, subcellular, or molecular level. In vitro cell culture methods commonly indicate basic cellular toxicity through measurement of membrane integrity, metabolic activities, and subcellular perturbations. Commonly used indicators for membrane integrity include cell viability (cell count), clonal expansion tests, trypan blue exclusion, intracellular enzyme release (e.g. lactate dehydrogenase), membrane permeability of small ions (K¹, Ca²⁺), and intracellular Ala accumulation of small molecules (e.g., ⁵¹Cr, succinate). Subcellular perturbations include monitoring mitochondrial enzyme activity levels via, for example, the MTT test, determining cellular adenine triphosphate (ATP) levels, neutral red uptake into lysosomes, and quantification of total protein synthesis. Metabolic activity indicators include glutathione content, lipid peroxidation, and lactate/pyruvate ratio.

### C. MTT Assay

The MTT assay is a fast, accurate, and reliable methodology for obtaining cell viability measurements. The MTT assay was first developed by Mosmann (Mosmann, J. Immunol. Meth., 65:55 (1983)). It is a simple calorimetric assay numerous laboratories have utilized for obtaining toxicity results (See e.g., Kuhlmann et al., Arch. Toxicol., 72:536 (1998)). Briefly, the mitochondria produce ATP to provide sufficient energy for the cell. In order to do this, the mitochondria metabolize pyruvate to produce acetyl CoA. Within the mitochondria, acetyl CoA reacts with various enzymes in the tricarboxylic acid cycle resulting in subsequent production of ATP. One of the enzymes particularly useful in the MTT assay is succinate dehydrogenase. MTT (3-(4,5-dimethylthiazol-2-yl)-2 diphenyl tetrazolium bromide) is a yellow substrate that is cleaved by succinate dehydrogenase forming a purple formazan product. The alteration in pigment identifies changes in mitochondria function. Nonviable cells are unable to produce formazan, and therefore, the amount produced directly correlates to the quantity of viable cells. Absorbance at 540 nm is utilized to measure the amount of formazan product.

The results of the in vitro tests can be compared to in vivo toxicity tests in order to extrapolate to live animal conditions (See, e.g., Example 13). Typically, acute toxicity from a single dose of the substance is assessed. Animals are monitored over 14 days for any signs of toxicity (increased temperature, breathing difficulty, death, etc). Traditionally, the standard of acute toxicity is the median lethal dose (LD₅₀), which is the predicted dose at which half of the treated population would be killed. The determination of this dose occurs by exposing test animals to a geometric series of doses under controlled conditions. Other tests include subacute toxicity testing, which measures the animal's response to repeated doses of the nanodevice for no longer than 14 days. Subchronic toxicity testing involves testing of a repeated dose for 90 days. Chronic toxicity testing is similar to subchronic testing but may last for over a 90-day period. In vivo testing can also be conducted to determine toxicity with respect to certain tissues. For example, in some embodiments of the present invention tumor toxicity (i.e., effect of the compositions of the present invention on the survival of tumor tissue) is determined (e.g., by detecting changes in the size and/or growth of tumor tissues).

### IX. Gene Therapy Vectors

In particular embodiments of the present invention, the dendrimer compositions comprise transgenes for delivery and expression to a target cell or tissue, in vitro, ex vivo, or in vivo. In such embodiments, rather than containing the actual protein, the dendrimer complex comprises an expression vector construct containing, for example, a heterologous DNA encoding a gene of interest and the various regulatory elements that facilitate the production of the particular protein of interest in the target cells.

In some embodiments, the gene is a therapeutic gene that is used, for example, to treat cancer, to replace a defective gene, or a marker or reporter gene that is used for selection or monitoring purposes. In the context of a gene therapy vector, the gene may be a heterologous piece of DNA. The heterologous DNA may be derived from more than one source (i.e., a multigene construct or a fusion protein). Further, the heterologous DNA may include a regulatory sequence derived from one source and the gene derived from a different source.

Tissue-specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, promoters may be used to target gene expression in other tissues (e.g., insulin, elastin amylase, pdr-1, pdx-1 and glucokinase promoters target to the pancreas; albumin PEPCK, HBV enhancer, alpha fetoproteinapolipoprotein C, alpha-1 antitrypsin, vitellogenin, NF-AB and transthyretin promoters target to the liver; myosin H chain, muscle creatine kinase, dystrophin, calpain p94, skeletal alpha-actin, fast troponin 1 promoters target to skeletal muscle; keratin promoters target the skin; sm22 alpha; SM-.alpha.-actin promoters target smooth muscle; CFTR; human cytokeratin 18 (K18); pulmonary surfactant proteins A, B and Q CC-10; P1 promoters target lung tissue; endothelin-1; E-selectin; von Willebrand factor; KDR/flk-1 target the endothelium; tyrosinase targets melanocytes).

The nucleic acid may be either cDNA or genomic DNA. The nucleic acid can encode any suitable therapeutic protein. Preferably, the nucleic acid encodes a tumor suppressor, cytokine, receptor, inducer of apoptosis, or differentiating agent. The nucleic acid may be an antisense nucleic acid. In such embodiments, the antisense nucleic acid may be incorporated into the nanodevice of the present invention outside of the context of an expression vector.

In preferred embodiments, the nucleic acid encodes a tumor suppressor, cytokines, receptors, or inducers of apoptosis. Suitable tumor suppressors include BRCA1, BRCA2, C-CAM, p16, p211 p53, p73, or Rb. Suitable cytokines include GMCSF, IL-1, IL-2, IL-3, IL-4, IL-5, IL6, IL-7, IL-8, IL-9, IL-10, IL-11, IL-12, IL-13, IL-14, IL-15, β-interferon, γ-interferon, or TNF. Suitable receptors include CFTR, EGFR, estrogen receptor, IL-2 receptor, or VEGFR. Suitable inducers of apoptosis include AdE1B, Bad, Bak, Bax, Bid, Bik, Bim, Harakiri, or ICE-CED3 protease.

### X. Methods of Combined Therapy

Tumor cell resistance to DNA damaging agents represents a major problem in clinical oncology. The nanodevices of the present invention provide means of ameliorating this problem by effectively administering a combined therapy approach. However, it should be noted that traditional combination therapy may be employed in combination with the nanodevices of the present invention. For example, in some embodiments of the present invention, nanodevices may be used before, after, or in combination with the traditional therapies.

To kill cells, inhibit cell growth, or metastasis, or angiogenesis, or otherwise reverse or reduce the malignant phenotype of tumor cells using the methods and compositions of the present invention in combination therapy, one contacts a “target” cell with the nanodevices compositions described herein and at least one other agent. These compositions are provided in a combined amount effective to kill or inhibit proliferation of the cell. This process may involve contacting the cells with the immunotherapeutic agent and the agent(s) or factor(s) at the same time. This may be achieved by contacting the cell with a single composition or pharmacological formulation that includes both agents, or by contacting the cell with two distinct compositions or formulations, at the same time, wherein one composition includes, for example, an expression construct and the other includes a therapeutic agent.

Alternatively, the nanodevice treatment may precede or follow the other agent treatment by intervals ranging from minutes to weeks. In embodiments where the other agent and immunotherapy are applied separately to the cell, one would generally ensure that a significant period of time did not expire between the time of each delivery, such that the agent and nanodevice would still be able to exert an advantageously combined effect on the cell. In such instances, it is contemplated that cells are contacted with both modalities within about 12-24 hours of each other and, more preferably, within about 6-12 hours of each other, with a delay time of only about 12 hours being most preferred. In some situations, it may be desirable to extend the time period for treatment significantly, however, where several days (2 to 7) to several weeks (1 to 8) lapse between the respective administrations.

In some embodiments, more than one administration of the immunotherapeutic composition of the present invention or the other agent are utilized. Various combinations may be employed, where the dendrimer is “A” and the other agent is “B”, as exemplified below:

A/B/A, B/A/B, B/B/A, A/A/B, B/A/A, A/B/B, B/B/B/A,
B/B/A/B, A/A/B/B, A/B/A/B, A/B/B/A, B/B/A/A,
B/A/B/A, B/A/A/B, B/B/B/A, A/A/A/B, B/A/A/A,
A/B/A/A, A/A/B/A, A/B/B/B, B/A/B/B, B/B/A/B.

Other combinations are contemplated. Again, to achieve cell killing, both agents are delivered to a cell in a combined amount effective to kill or disable the cell.

Other factors that may be used in combination therapy with the nanodevices of the present invention include, but are not limited to, factors that cause DNA damage such as .gamma.-rays, X-rays, and/or the directed delivery of radioisotopes to tumor cells. Other forms of DNA damaging factors are also contemplated such as microwaves and UV-irradiation. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In preferred embodiments of the present invention, the regional delivery of the nanodevice to patients with cancers is utilized to maximize the therapeutic effectiveness of the delivered agent. Similarly, a nanodevice comprising one or more functional groups (e.g., a therapeutic agent such as a chemotherapeutic or radiotherapeutic) may be directed to particular, affected region of the subjects body. Alternatively, systemic delivery of a nanodevide (e.g., a dendrimer comprising a therapeutic agent, targeting agent, and/or imaging agent) may be appropriate in certain circumstances, for example, where extensive metastasis has occurred, or where metastasis is suspected.

In addition to combining the nanodevice with chemo- and radiotherapies, it also is contemplated that traditional gene therapies are used. For example, targeting of p53 or p16 mutations along with treatment of the nanodevices provides an improved anti-cancer treatment. The present invention contemplates the co-treatment with other tumor-related genes including, but not limited to, p21, Rb, APC, DCC, NF-I, NF-2, BCRA2, p16, FHIT, WT-I, MEN-I, MEN-II, BRCA1, VHL, FCC, MCC, ras, myc, neu, raf erb, src, fms, jun, trk, ret, gsp, hst, bcl, and abl.

In vivo and ex vivo treatments are applied using the appropriate methods worked out for the gene delivery of a particular construct for a particular subject. For example, for viral vectors, one typically delivers 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies.

An attractive feature of the present invention is that the therapeutic compositions may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (Scimed Life Systems, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

### XI. Photodynamic Therapy

In some embodiments, the therapeutic complexes of the present invention comprise a photodynamic compound and a targeting agent that is administered to a patient. In some embodiments, the targeting agent is then allowed a period of time to bind the “target” cell (e.g. about 1 minute to 24 hours) resulting in the formation of a target cell-target agent complex. In some embodiments, the therapeutic complexes comprising the targeting agent and photodynamic compound are then illuminated (e.g., with a red laser, incandescent lamp, X-rays, or filtered sunlight). In some embodiments, the light is aimed at the jugular vein or some other superficial blood or lymphatic vessel. In some embodiments, the singlet oxygen and free radicals diffuse from the photodynamic compound to the target cell (e.g. cancer cell or pathogen) causing its destruction.

### XII. Pharmaceutical Formulations

Where clinical applications are contemplated, in some embodiments of the present invention, the nanodevices are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight dendrimer formulation may be administered using one or more of the routes described herein.

In preferred embodiments, the nanodevices are used in conjunction with appropriate salts and buffers to render delivery of the compositions in a stable manner to allow for uptake by target cells. Buffers also are employed when the nanodevices are introduced into a patient. Aqueous compositions comprise an effective amount of the nanodevice to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active nanodevices may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

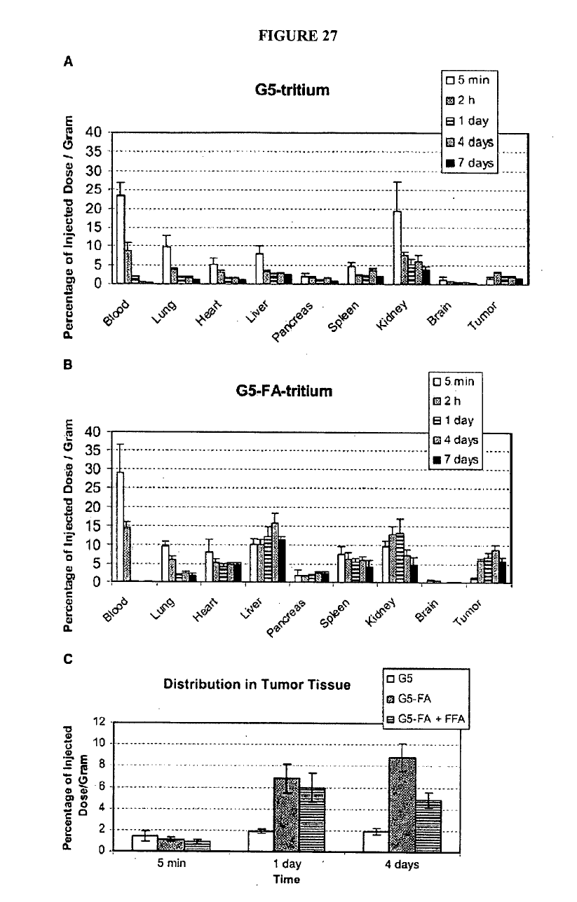

In some embodiments, the present invention provides a composition comprising a dendrimer comprising a targeting agent, a therapeutic agent and an imaging agent. In preferred embodiments, the dendrimer is used for delivery of a therapeutic agent (e.g., methotrexate) to tumor cells in vivo (See, e.g., Example 13, FIG. 27). In some embodiments, the therapeutic agent is conjugated to the dendrimer via an acid-labile linker. Thus, in some embodiments, the therapeutic agent is released from the dendrimer within a target cell (e.g., within an endosome). This type of intracellular release (e.g., endosomal disruption of the acid-labile linker) is contemplated to provide additional specificity for the compositions and methods of the present invention. In preferred embodiments, the dendrimers of the present invention (e.g., G5 PAMAM dendrimers) contain between 100-150 primary amines on the surface (See, e.g., Example 13). Thus, the present invention provides dendrimers with multiple (e.g., 100-150) reactive sites for the conjugation of functional groups comprising, but not limited to, therapeutic agents, targeting agents, imaging agents and biological monitoring agents.

The compositions and methods of the present invention are contemplated to be equally effective whether or not the dendrimer compositions of the present invention comprise a fluorescein (e.g. FITC) imaging agent (See, e.g., Example 13). Thus, each functional group present in a dendrimer composition is able to work independently of the other functional groups. Thus, the present invention provides a dendrimer that can comprise multiple combinations of targeting, therapeutic, imaging, and biological monitoring functional groups.

The present invention also provides a very effective and specific method of delivering molecules (e.g., therapeutic and imaging functional groups) to the interior of target cells (e.g., cancer cells). Thus, in some embodiments, the present invention provides methods of therapy that comprise or require delivery of molecules into a cell in order to function (e.g., delivery of genetic material such as siRNAs).

In some embodiments, pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, the dendrimer compositions are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The nanodevices also may be formulated as inhalants for the treatment of lung cancer and such like.

### XIII. Method of Treatment or Prevention of Cancer and Pathogenic Diseases

In specific embodiments of the present invention methods and compositions are provided for the treatment of tumors in cancer therapy (See, e.g., Example 13). It is contemplated that the present therapy can be employed in the treatment of any cancer for which a specific signature has been identified or which can be targeted. Cell proliferative disorders, or cancers, contemplated to be treatable with the methods of the present invention include, but are not limited to, human sarcomas and carcinomas, including, but not limited to, fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

It is contemplated that the present therapy can be employed in the treatment of any pathogenic disease for which a specific signature has been identified or which can be targeted for a given pathogen. Examples of pathogens contemplated to be treatable with the methods of the present invention include, but are not limited to, Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Staphylococcus aureus, human papilloma virus, human immunodeficiency virus, rubella virus, polio virus, and the like.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: g (grams); l or L (liters); μg (micrograms); μl (microliters); μm (micrometers); μM (micromolar); μmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); pmol (picomoles); Aldrich (Sigma/Aldrich, Milwaukee, Wis.); Sigma (Sigma Chemical Co., St. Louis, Mo.); Fisher Scientific (Fisher Scientific, Pittsburgh, Pa.); Millipore (Millipore, Billerica, Mass.); Mettler Toledo Mettler Toledo (Columbus, Ohio); Waters (Waters Corporation, Milford, Mass.); Wyatt Technology (Wyatt Technology Corp., Santa Barbara, Calif.); TosoHaas (TosoHaas Corp., Montgomeryville, Pa.); Perkin Elmer (Perkin Elmer, Wellesley, Mass.); Beckman Coulter (Beckman Coulter Corp., Fullerton, Calif.); Phenomenex (Phenomenex, Torrance, Calif.); GiboBRL (GibcoBRL/Life Technologies, Gaithersburg, Md.); Pierce (Pierce Chemical Company, Rockford, Ill.); Roche (F. Hoffmann-La Roche Ltd, Basel, Switzerland).

### Example 1

### Materials and Methods

The G5 PAMAM dendrimer was synthesized and characterized at the Center for Biologic Nanotechnology, University of Michigan. MeOH(HPLC grade), acetic anhydride (99%), triethylamine (99.5%), DMSO (99.9%), fluorescein isothiocyanate (98%), glycidol (racemic form, 96%), DMF (99.8%), 1-(3-(Dimethylamino)-propyl)-3-ethylcarbodiimide HCl (EDC, 98%), citric acid (99.5%), sodium azide (99.99%), D₂O, NaCl, and volumetric solutions (0.1M HCl and 0.1M NaOH) for potentiometric titration were all purchased from Aldrich and used as received. Methotrexate (99+%) and Folic Acid (98%) were from Sigma, Spectra/Por® dialysis membrane (MWCO 3,500), Millipor Centricon ultrafiltration membrane YM-10, and phosphate buffer saline (PBS, pH=7.4) were from Fisher Scientific.

Potentiometric Titration. Titration was carried out manually using a Mettler Toledo MP230 pH Meter and MicroComb pH electrode at room temperature, 23±1° C. A 10 mL solution of 0.1 M NaCl was added to precisely weighed 100 mg of PAMAM dendrimer to shield amine group interactions. Titration was performed with 0.1028 N HCl, and 0.1009 N NaOH was used for back titration. The numbers of primary and tertiary amines were determined from back titration data.

Gel Permeation Chromatography (GPC). GPC experiments were performed on an Alliance Waters 2690 Separation Module equipped with 2487 Dual Wavelength UV Absorbance Detector (Waters), a Wyatt Dawns DSP Laser Photometer, an Optilab DSP Interferometric Refractometer (Wyatt Technology), and with TosoHaas TSK-Gel® Guard PHW 06762 (75×7.5 mm, 12 μm), G 2000 PW 05761 (300×7.5 mm, 10 μm), G 3000 PW 05762 (300×7.5 mm, 10 μm), and G 4000 PW (300×7.5 mm, 17 μm) columns. Column temperature was maintained at 25±0.1° C. by a Waters Temperature Control Module. The isocratic mobile phase was 0.1 M citric acid and 0.025 w % sodium azide, pH 2.74, at a flow rate of 1 ml/min. Sample concentration was 10 mg/5 ml with an injection volume of 100 μL. Molecular weight, and molecular weight distribution of the PAMAM dendrimer and its conjugates were determined using Astra 4.7 software (Wyatt Technology).

Nuclear Magnetic Resonance Spectroscopy: 1H and ¹³C NMR spectra were taken in D₂O and were used to provide integration values for structural analysis by means of a Bruker AVANCE DRX 500 instrument.

UV Spectrophotometry. UV spectra were recorded using Perkin Elmer UV/VIS Spectrometer Lambda 20 and Lambda 20 software, in PBS.

Reverse Phase High Performance Liquid Chromatography. A reverse phase ion-pairing high performance liquid chromatography (RP-HPLC) system consisted of a System GOLD™ 126 solvent module, a Model 507 auto sampler equipped with a 100 μl loop, and a Model 166 UV detector (Beckman Coulter). A Phenomenex Jupiter C5 silica based HPLC column (250×4.6 mm, 300 Å) was used for separation of analytes. Two Phenomenex Widepore C5 guard columns (4×3 mm) were also installed upstream of the HPLC column. The mobile phase for elution of PAMAM dendrimers was a linear gradient beginning with 90:10 water/acetonitrile (ACN) at a flow rate of 1 ml/min, reaching 50:50 after 30 minutes. Trifluoroacetic acid (TFA) at 0.14 w % concentration in water as well as in ACN was used as counter-ion to make the dendrimer-conjugate surfaces hydrophobic. The conjugates were dissolved in the mobile phase (90:10 water/ACN). The injection volume in each case was 50 μl with a sample concentration of approximately 1 mg/ml and the detection of eluted samples was performed at 210, or 242, or 280 nm. The analysis was performed using Beckman's System GOLD™ Nouveau software. Characterization of each device and all intermediates has been performed through the use of UV, HPLC, NMR, and GPC.

The KB cells were obtained from ATCC (CLL17; Rockville, Md.). Trypsin-EDTA, Dulbecco's phosphate-buffered saline (PBS), fetal bovine serum, cell culture antibiotics and RPMI medium were obtained from Gibco/BRL. All other reagents were from Sigma. The synthesis and characterization of the dendrimer-conjugates is reported as a separate communication. All the dendrimer preparations used in this study were synthesized at our center and have been surface neutralized by acetylation of the free surface amino groups.

Cell culture and treatment. KB cells were maintained in folate-free medium containing 10% serum (See, e.g., Quintana et al, Pharm. Res. 19, 1310 (2002)) to provide extracellular FA similar to that found in human serum. Cells were plated in 12-well plates for uptake studies, in 24-well plates for cell growth analysis, and in 96-well plates for XTT assay. Cells were rinsed with FA-free medium containing dialyzed serum and incubated at 37° C. with dendrimer-drug conjugates for the indicated time periods and concentrations. KB cells were also maintained in RPMI medium containing 2 μM FA to obtain cells which express low FAR.

Flow Cytometry and Confocal Microscopy. The standard fluorescence of the dendrimer solutions was quantified using a Beckman spectrofluorimeter. For flow cytometric analysis of the uptake of the targeted polymer, cells were trypsinized and suspended in PBS containing 0.1% bovine serum albumin (PBSB) and analyzed using a Becton Dickinson FACScan analyzer. The FL1-fluorescence of 10,000 cells was measured and the mean fluorescence of gated viable cells was quantified. Confocal microscopic analysis was performed in cells plated on a glass cover-slip, using a Carl Ziess confocal microscope. Fluorescence and differential interference contrast (DIC) images were collected simultaneously using an argon laser, using the appropriate filters for FITC.

Evaluation of dendrimer cytotoxicity. Cell growth was determined by assay of the total protein in lysates of treated cells using a bicinchoninic acid reagent (PIERCE, and by XTT assay, using a kit from Roche.

### Example 2

### Syntheses of Dendrimer

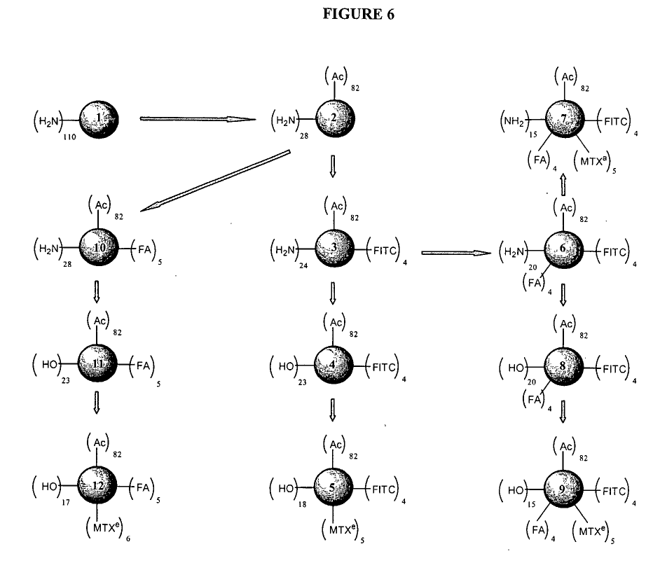

Dendrimers were synthesized according to the following process (See, e.g., FIG. 6):

1. G5 carrier 7. G5-Ac¹(82)-FITC-FA-MTX^{a}

2. G5-Ac³(82) 8. G5-Ac³(82)-FITC-FA-OH

3. G5-Ac³(82)-FITC 9. G5-Ac³(82)-FITC-FA-OH-MTX^{e}

4. G5-Ac³(82)-FITC-OH 10. G5-Ac²(82)-FA

5. G5-Ac³(82)-FITC-OH-MTX^{e} 11. G5-Ac²(82)-FA-OH

6. G5-Ac³(82)-FITC-FA 12. G5-Ac²(82)-FA-OH-MTX^{e}

(Note: The superscripts indicated in Ac¹, Ac². Ac³ are utilized to differentiate different sets of acetylation reactions).

1. G5 carrier. The PAMAM G5 dendrimer was synthesized and characterized at the Center for Biologic Nanotechnology, University of Michigan. PAMAM dendrimers are composed of an ethylenediamine (EDA) initiator core with four radiating dendron arms, and are synthesized using repetitive reaction sequences comprised of exhaustive Michael addition of methyl acrylate (MA) and condensation (amidation) of the resulting ester with large excesses of EDA to produce each successive generation. Each successive reaction therefore theoretically doubles the number of surface amino groups, which can be activated for functionalization. The synthesized dendrimer has been analyzed and the molecular weight has been found to be 26,380 g/mol by GPC and the average number of primary amino groups has been determined by potentiometric titration to be 110.

2. G5-Ac³(82). 2.38696 g (8.997*10⁻⁵ mol) of G5 PAMAM dendrimer (MW=26,380 g/mol by GPC, number of primary amines=110 by potentiometric titration) in 160 ml of abs. MeOH was allowed to react with 679.1 μl (7.198*10⁻³ mol) of acetic anhydride in the presence of 1.254 ml (8.997*10⁻³ mol, 25% molar excess) triethylamine. After intensive dialysis and lyophilization 2.51147 g (93.4%) G5-Ac³(82) product was yielded. The average number of acetyl groups (82) has been determined based on ¹H NMR calibration (Majoros, I. J., Keszler, B., Woehler, S., Bull, T., and Baker, J. R., Jr. (2003)).

3. G5-Ac³(82)-FITC. 1.16106 g (3.884*10⁻⁵ mol) of G5-Ac³(82) partially acetylated PAMAM (MW=29,880 g/mol by GPC) in 110 ml of abs. DMSO was allowed to react with 0.08394 g (90% pure) (1.94*10⁻⁴ mol) of FITC under nitrogen overnight. After intensive dialysis, lyophilization 1.10781 g, (89.58%) G5-Ac³(82)-FITC product was yielded. Further purification was done through membrane filtration.

4. G5-Ac³(82)-FITC-OH. 0.20882 g (6.51*10⁻⁶ mol) of G5-Ac³(82)-FITC was allowed to react with 19.9 μl (2.99*10⁻⁴ mol) of glycidol (racemic) in 150 ml of DI water. Two glycidol molecules were calculated for each remaining primary amino group. The reaction mixture was stirred vigorously for 3 hrs at room temperature. After intensive dialysis for 2 days, and lyophilization, the yield of the product G5-Ac³(82)-FITC-OH was 0.18666 g (84.85%).

5. G5-Ac³(82)-FITC-OH-MTX^{e}. 0.02354 g MTX (5.18*10⁻⁵ mol) was allowed to react with 0.13269 g (6.92*10⁻⁴ mol) EDC in 27 ml DMF and 9 ml DMSO for 1 hr at room temperature with vigorous stirring. This solution was added drop wise to 150 ml DI water solution containing 0.09112 g (2.72*10⁻⁶ mol) of G5-Ac³(82)-FITC-OH. The reaction was vigorously stirred for 3 days at room temperature. After intense dialysis, and lyophilization, the yield of the targeted molecule G5-Ac³(82)-FITC-OH-MTX^{e} was 0.08268 g (73.5%).

6. G5-Ac³(82)-FITC-FA. 0.03756 g (8.51*10⁻⁵ mol) of FA (MW=441.4 g/mol) was allowed to react with 0.23394 g (1.22*10⁻³ mol) of EDC (1-(3-(dimethylamino)-propyl)-3-ethylcarbodiimide HCl; MW=191.71 g/mol) in 27 ml dry DMF and 9 ml dry DMSO mixture under nitrogen atmosphere for 1 hr. Then this organic reaction mixture was added drop wise to the DI water solution (100 ml) of 0.49597 g (1.55*10⁻⁵ mol; MW=32,150 g/mol) G5-Ac³(82)-FITC. The reaction mixture was vigorously stirred for 2 days. After dialysis and lyophilization G5-Ac³(82)-FITC-FA weight was 0.5202 g (98.1%). Further purification was done through membrane filtration.

7. G5-Ac¹(82)-FITC-FA-MTX^{a}. 2.1763*10⁻⁵ mol (0.00989 g) of MTX (MW=454.45 g/mol) was allowed to react with 3.0948*10⁻⁴ mol (0.05933 g) of EDC in 66 ml dry DMF and 22 ml dry DMSO mixture under nitrogen atmosphere for 1 hr. This organic reaction mixture was added drop wise to the DI water solution (260 ml) of 0.09254 g (2.7051*10⁻⁶ mol; MW=34,710 g/mol by GPC) G5-Ac¹(82)-FITC-FA-NH₂. The solution was vigorously stirred for 2 days. After dialysis and lyophilization G5-Ac¹(82)-FITC-FA-MTX^{a} weight was 0.09503 g (96.5%). Further purification was done by membrane filtration before analysis. This three-functional device served as a control compound in drug cleavage in in vitro cytotoxicity study.

8. G5-Ac³(82)-FITC-FA-OH. 0.29597 g (8.63*10⁻⁶ mol) of G5-Ac³(82)-FITC-FA partially acetylated PAMAM dendrimer conjugate (MW=34,710 g/mol by GPC) in 200 ml of DI water was allowed to react with 20.6 μl (3.1*10⁻⁴ mol, 25% molar excess) of glycidol (MW=74.08 g/mol) for 3 hrs. After intensive dialysis, lyophilization and repeated membrane filtration 0.27787 g (90.35%) fully glycidylated G5-Ac³(82)-FITC-FA-OH product was yielded. Non-specific uptake was not observed in in vitro study (see Part II of this research for uptake study (24).

9. G5-Ac³(82)-FITC-FA-OH-MTX^{e}. 0.03848 g (8.4674*10⁻⁵ mol) of MTX (MW=454.45 g/mol) was allowed to react with 0.22547 g (1.176*10⁻³ mol) of EDC in 54 ml dry DMF and 18 ml dry DMSO mixture under nitrogen atmosphere for 1 hr. This organic reaction mixture was added drop wise to the DI water solution (240 ml) of 0.16393 g (4.6339*10⁻⁶ mol; MW=36,820 g/mol) G5-Ac³(82)-FITC-FA-OH. The solution was vigorously stirred for 3 days. After dialysis, repeated membrane filtration and lyophilization G5-Ac³(82)-FITC-FA-MTX^{e} weight was 0.18205 g (90.88%).

10. G5-Ac²(82)-FA. FA was attached to G5-Ac²(82) in two consecutive reactions. 0.03278 g (7.426*10⁻⁵ mol) FA was allowed to react with a 14-fold excess of EDC 0.19979 g (1.042*10⁻³ mol) in a 24 ml DMF, 8 ml DMSO solvent mixture at room temperature, then this FA-active ester solution was added drop wise to an aqueous solution of the partially acetylated product G5-Ac²(82) (0.40366 g, 1.347*10⁻⁵ mol) in 90 ml water. After dialysis and lyophilization, the product weight was 0.41791 g (96.7%). The number of FA molecules was determined by UV spectroscopy. As an additional characterization, no free FA was observed by a GPC equipped with a UV detector, or by agarose gel.

11. G5-Ac²(82)-FA-OH. 0.21174 g (6.60*10⁻⁶ mol) of mono-functional dendritic device, G5-Ac²(82) —FA, was allowed to react with 20.1 μl (3.04*10⁻⁴ mol) of glycidol in 154 ml DI water under vigorous stirring for 3 hrs. After dialysis and lyophilization the glycidolated mono-functional device, having hydroxyl groups on the surface (yield: 0.20302 g, 91.05%), participated in the conjugation reaction with methotrexate.

12. G5-Ac²(82)-FA-OH-MTX^{e}. In 27 ml of DMF and 9 ml of DMSO solvent mixture, 0.02459 g (5.41*10⁻⁵ mol) of MTX and 0.14315 g (7.46*10⁻⁴ mol) of 1-(3-(dimethylamino)-propyl)-3-ethylcarbodiimide hydrochloride (EDC) was allowed to react under nitrogen at room temperature for 1 hr. The reaction mixture was vigorously stirred. The MTX-active ester solution was added drop wise to the 0.09975 g (2.95*10⁻⁶ mol) of mono-functional dendritic device, having hydroxyl groups on the surface, in 150 ml DI water, and this reaction mixture was stirred at room temperature for 3 days. After dialysis and lyophilization this bi-functional device G5-Ac²(82)-FA-OH-MTX^{e} (yield: 0.11544 g, 93.9%) was tested by compositional and biological matter.

### Example 3

### Potentiometric Titration Curves to Analyze Terminal Primary Amino Groups of G5 PAPAM Dendrimer

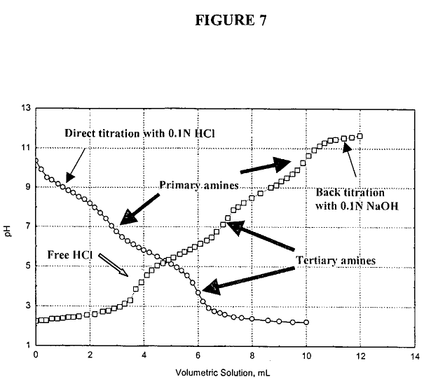

Potentiometric titration was performed to determine the number of primary and tertiary amino groups. Theoretically, the G5 PAMAM dendrimer has 128 primary amine groups on its surface, and 126 tertiary amine groups. These values can be determined through use of mathematical models. Potentiometric titration revealed that there were 110 primary amines present on the surface of the G5 PAMAM dendrimer carrier (See, e.g., FIG. 7, which shows the titration curves performed by direct titration with 0.1 M HCl volumetric solution and back-titration with 0.1 M NaOH volumetric solution). The average number of primary amino groups was calculated using back titration data performed with 0.1M NaOH volumetric solution.

The determination of molecular weight of each conjugate structure was also necessary in order to produce a well-defined multi-functional dendrimer. GPC equipped with multi-angle laser light scattering and an RI detector as a concentration detector was used for this purpose (See, e.g., Table 1, which presents PAMAM dendrimer carrier and its mono-, bi-, and tri-functional conjugates with molecular weights and molecular weight distribution given for each. The superscript numerals 2 and 3 (ex.—G5-Ac² and G5-Ac³) indicate that these are two independent acetylation reactions).

TABLE 1

| | M̅ₙ̅, g/mol | M̅_̅{̅w̅}̅, g/mol | M̅_̅{̅w̅}̅/ M̅ₙ̅ |
|---|---|---|---|
| G5 | 26,380 | 26,890 | 1.020 |
| G5-Ac² | 29,830 | 30,710 | 1.030 |
| G5-Ac²-FA | 32,380 | 35,470 | 1.095 |
| G5-Ac²-FA-OH | 34,460 | 40,580 | 1.178 |
| G5-Ac²-FA-OH-MTX^{e} | 36,730 | 36,960 | 1.006 |
| G5-Ac³ | 29,880 | 30,760 | 1.030 |
| G5-Ac³-FITC | 32,150 | 32,460 | 1.100 |
| G5-Ac³-FITC-OH | 34,380 | 34,790 | 1.012 |
| G5-Ac³-FITC-OH-MTX^{e} | 37,350 | 37,800 | 1.012 |
| G5-Ac³-FITC-FA | 34,710 | 35,050 | 1.010 |
| G5-Ac³-FITC-FA-OH | 36,820 | 37,390 | 1.016 |
| G5-Ac³-FITC-FA-OH-MTX^{e} | 39,550 | 39,870 | 1.008 |

### Example 4

### Dendrimer Characterization Via Gel Permeation Chromatography

The measured molecular weight of the G5 dendrimer of 26,380 g/mol is slightly lower than the theoretical one, (28,826 g/mol). These results indicate a deviation from the theoretical structure. The values in Table 1 were calculated utilizing GPC data for each conjugate (See, e.g., FIG. 8) and were calculated in order to derive the precise number of each functional group attached to the carrier. The average number of each functional molecule can be calculated by subtracting the M̅ₙ̅ value of the conjugate without the functional molecule in question from the M̅ₙ̅ value of the conjugate containing the functional molecule, and dividing by the molecular weight of the functional molecule. GPC eluograms of G5-Ac², G5-Ac²(82)-FA-OH-MTX^{e}, G5-Ac³(82)-FITC-OH-MTX^{e}, and G5-Ac²(82)-FITC-FA-OH-MTX^{e}, can be presented, with the RI signal and laser light scattering signal overlapping at 90° (See, e.g., FIG. 8).

Based on GPC analysis, the number of conjugated FITC, FA, MTX, and glycidol molecules can be determined (See, e.g., FIG. 8: FITC: 5.8, FA: 5.7, MTX^{e}: 5-6, OH: 28-30). The number of conjugated molecules as determined by GPC was slightly higher than assumed; this is most probably due to the effect of citric acid in the eluent, which has varying effects dependent on the device in question. These values along with values obtained through analysis of NMR and UV spectra are utilized in combination to precisely determine the number of each conjugate molecules attached to the dendrimer.

Theoretical and defected chemical structures of the G5 PAMAM dendrimer are presented (See, e.g., FIG. 9). Side reactions such as bridging, as well as production of fewer arms per generation than theoretically expected, aid in producing a structure slightly different from the theoretical representation of the G5 PAMAM dendrimer. The defected chemical structure of a G5 PAMAM dendrimer exhibits missing arms from each generation, which can become problematic because they disturb the globular shape of the dendrimer, therefore affecting the number of functional molecules it is possible to attach and lessening the effects each functional molecule can have within the targeted cell(s).

### Example 5

### Characterization of Dendrimer Functional Groups

Acetylation of the dendrimer. Acetylation is the first requisite step in the synthesis of dendrimers. Partial acetylation is used to neutralize a fraction of the dendrimer surface from further reaction or intermolecular interaction within the biological system, therefore preventing non-specific interactions from occurring during synthesis and during drug delivery. Leaving a fraction of the surface amines non-acetylated allows for attachment of functional groups. Acetylation of the remaining amino groups results in increased water solubility (after FITC conjugation), allowing the dendrimer to disperse more freely within aqueous media with increased targeting specificity, giving it greater potential for use as a targeted delivery system as compared to many conventional mediums (Quintana et al., Pharm. Res. 19, 1310 (2002)).

Intensive dialysis, lyophilization and repeated membrane filtration using PBS and DI water were used to yield the purified, partially acetylated G5-Ac²(82) and G5-Ac³(82) PAMAM dendrimers (See, e.g., Majoros et al., Macromolec 36, 5526 (2003)). After conjugation of fluorescein isothiocyanate (FITC), and (FITC-FA) the dendrimer was fully acetylated again for an in vitro uptake study, following the same reaction sequence as found in (Wang, et al., Blood. 15, 3529 (2000)). Intensive dialysis, lyophilization and repeated membrane filtration were performed, yielding the fully acetylated G5-Ac¹(82)-FITC and G5-Ac¹(82)-FITC-FA PAMAM.

As the degree of acetylation rises, the diameter of the dendrimer decreases, demonstrating an inverse relationship between the degree of acetylation and dendrimer diameter (See, e.g., Majoros et al., Macromolec 36, 5526 (2003)). The lower number of primary amines available for protonation (at a higher degree of acetylation, as compared to a lower degree) leads to a structure less impacted by charge-charge interactions, therefore leading to a more compact structure. The molecular weight however, has a parallel relationship to the degree of acetylation, as molecular weight increases as the degree of acetylation rises.

The PAMAM dendrimer was further characterized by H¹-NMR and HPLC (See, e.g., FIGS. 10 (A) and (B), respectively), by monitoring the eluted fractions by UV detection at 210 nm. H¹-NMR spectrum for G5-Ac displays the following: the peak appearing at 4.71 ppm is representative of D₂O, the peak at 3.67 ppm is representative of the external standard dioxane, and the peak at 1.89 ppm represents the methyl protons of the acetamide. Peaks 2.34 ppm, 2.55 ppm, 2.74 ppm, 3.04 ppm, 3.21 ppm, and 3.39 ppm are representative of the protons present in the acetylated dendrimer.

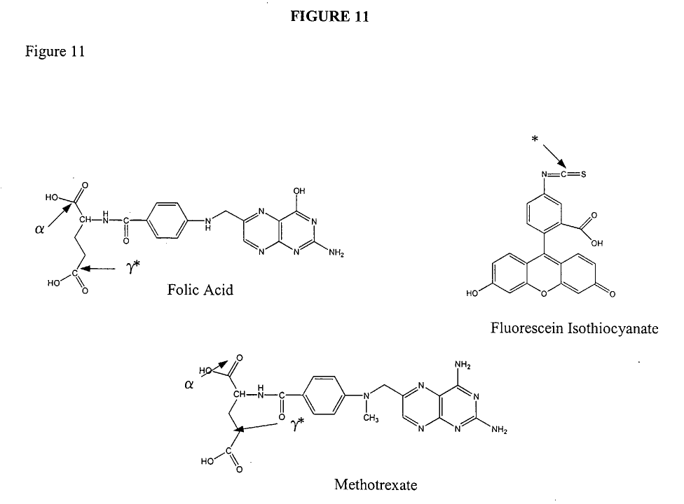

Structure of the functional groups. The structures of FITC, FA, and MTX are presented with the group to be attached to the dendrimer marked with an asterisk (See., e.g., FIG. 11, with the α- and γ-carboxyl groups labeled on both the FA and MTX molecules). When the γ-carboxylic group on FA is used for conjugation to the dendrimer, FA retains strong affinity towards its receptor, enabling FA to retain its ability to act as a targeting agent. Additionally, the γ-carboxylic group possesses higher reactivity during carbodiimide mediated coupling to amino groups as compared to the α-carboxyl group (See, e.g., Quintana, et al., Pharm. Res. 19, 1310 (2002)).

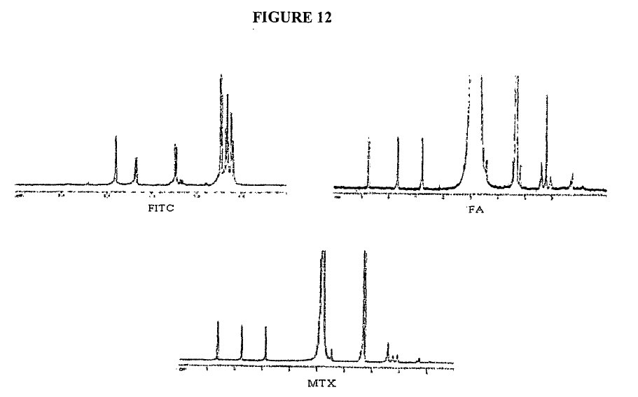

H¹-NMR of functional groups. In order to conclusively determine the numbers of each type of functional group attached to the dendrimer, the H-NMR of the functional groups themselves, and the H¹-NMR of the dendrimer conjugated to the functional groups must be compared. The H¹-NMR of the functional groups (See, for e.g., FIG. 12) the: FITC H¹-NMR—aromatic peaks: 7.9 ppm, 7.68 ppm, 7.23 ppm, 6.6 ppm, 6.65 ppm, 6.75 ppm; FA H¹-NMR—aromatic peaks: 8.73 ppm, 6.75 ppm, 7.65 ppm, D₂O at 4.85 ppm, CH₃OD at 3.3 ppm, aliphatic peaks at: 2.15 ppm, 2.2 ppm, 2.4 ppm; and MTX H¹-NMR—aromatic peaks: 8.65 ppm, 8.75 ppm, 7.85 ppm, D₂O at 4.8 ppm, CH₃OD at 3.35 ppm, aliphatic peaks at: 2.05 ppm, 2.25 ppm, 2.45 ppm.

### Example 6

### Conjugation of Functional Groups to Acetylated Dendrimer

Conjugation of fluorescein isothiocyanate to acetylated dendrimer. A partially acetylated G5-Ac³(82) PAMAM dendrimer was used for the conjugation of fluorescein isothiocyanate (FITC). The partially acetylated dendrimer was allowed to react with fluorescein isothiocyanate, and after intensive dialysis, lyophilization and repeated membrane filtration the G5-Ac³(82)-FITC product was yielded. The formed thiourea bond was stable during investigation of the devices.

Conjugation of folic acid to acetylated mono-functional dendrimer. Conjugation of folic acid to the partially acetylated mono-functional dendritic device was carried out via condensation between the γ-carboxyl group of folic acid and the primary amino groups of the dendrimer. This reaction mixture was added drop wise to a solution of DI water containing G5-Ac³(82)-FITC and was vigorously stirred for 2 days (under nitrogen atmosphere) to allow for the FA to fully conjugate to the G5-Ac³(82)-FITC. It is obvious that the α carboxyl group will participate in the condensation reaction, but its reactivity is much lower when compared to the γ carboxyl group. NMR was also used to confirm the number of FA molecules attached to the dendrimer. In the case that free FA is present within the sample, sharp peaks would appear in the spectrum. The ¹H NMR spectra of free FA (See, for e.g., FIG. 12) and G5-Ac³(82)-FITC-FA were taken. The broadening of the aromatic proton peaks in the G5-Ac³(82)-FITC-FA spectrum indicates the presence of a covalent bond between the FA and the dendrimer. Based on the integration values of the methyl protons in the acetamide groups and the aromatic protons in the FA, the number of attached FA molecules was calculated to be 4.5. The number of FA molecules (4.8), was determined by UV spectroscopy, utilizing the free FA concentration calibration curve.

Conjugation of MTX to acetylated two-functional dendrimer (via amide link). A control, MTX, tri-functional conjugate was synthesized from G5-Ac³(82)-FITC-FA. The similarity in structure of MTX, a commonly used anti-cancer drug, to FA allows for its attachment to G5-Ac³(82)-FITC-FA through the same condensation reaction used to attach FA to the primary amino groups. It was expected, from the molar ratio of the reactants, that five drug molecules would be attached per dendrimer. The ¹H NMR spectrum of the three-functional device was taken. The broadening of the aromatic proton peaks indicates the presence of a covalent bond between methotrexate and the dendrimer. Based on the integration values of the methyl protons in acetamide groups and the aromatic protons in the conjugated molecules, the number of attached methotrexate molecules was calculated to be five. MTX conjugation by an amide bond served as a control device for comparison of MTX conjugation through an ester bond. Attachment of methotrexate via an ester bond allows for relatively easier cleavage and release of the drug into the system as compared to linkage of MTX to the dendrimer by an amide bond.

Conjugation of glycidol to acetylated two-functional dendrimer. The conjugation of glycidol to the acetylated two-functional device was an important precursory step in order to attach MTX via an ester linkage and eliminate the remaining NH₂ to avoid any unwanted nonspecific targeting within the biological system. Conjugation of glycidol to the G5-Ac³(82)-FITC-FA converted all the remaining primary amino groups to alcohol groups, producing G5-Ac³(82)-FITC-FA-OH. For characterization purposes, conjugation of MTX to a glycidolated dendritic device containing FA or FITC produced G5-Ac²-FA-OH-MTX^{e1}* and G5-Ac³-FITC-OH-MTX^{e2}*(See, for e.g., FIGS. 13(A) and (B), the HPLC eluograms of each sample, respectively.).

### Example 7

### Characterization of MTX Conjugated to Acetylated and Glycidylated Two-Functional Dendrimer Via Ester Link

The H¹-NMR for G5-Ac²-FA-OH-MTX^{e} is shown (See, for e.g., FIG. 14). The peaks representative of the aromatic protons of the conjugated device are indistinguishable from the aromatic peaks found in the H¹-NMR of free FA and MTX. Aromatic protons appear doubly 6.59 ppm, 7.53 ppm, and singly at 8.37 ppm. Comparison of the H¹-NMR of free FA and free MTX with that of the conjugated device shows that the aromatic regions overlap almost identically, therefore making it impossible to determine the location of the aromatic protons. The number of attached molecules of FA and MTX also affects the distributions of the peaks. The peak appearing at 4.70 ppm represents the solvent D₂O, the peak appearing at 3.67 ppm is representative of the external standard dioxane, and the peak appearing at 1.89 ppm is representative of the methyl protons of the acetamide groups. Peaks 2.31 ppm, 2.52 ppm, 2.71 ppm, and 3.26 ppm are representative of protons of the dendrimer.

### Example 8

### UV Spectra Characterization of Dendrimers

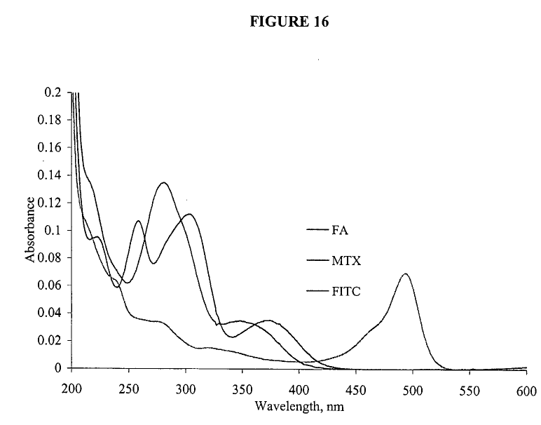

MTX conjugation via an ester linkage was tested for improved cleavage as compared to conjugation to the dendrimer via an amide linkage. The MTX is attached by use of EDC chemistry. The HPLC eluogram for G5-Ac-FITC-FA-OH-MTX at 305 nm is shown (See, for e.g., FIG. 15). The combined UV spectra for free FA, MTX and FITC can be compared to the for UV spectra of G5-Ac(82), mono-, bi- and tri-functional dendrimers (See, for e.g., FIGS. 16 and 17, respectively). UV spectra present defining peaks for FA at precisely 281 nm and 349 nm, for MTX on the order of 258 nm, 304 nm and 374 nm, and for FITC at 493 nm. The distinguishing peaks for FA, FITC and MTX visible (See, for e.g., FIG. 16) are dependent on the conjugation of each molecule to the dendrimer. Characterization of each device by comparison of UV spectra of free material and dendrimer-conjugated material was used to determine which function has been attached to the dendrimer.

### Example 9

### Cellular Uptake of Dendrimers

The fluorescence of the standard solutions of the conjugates G5-FI, G5-FITC-FA and G5-FITC-FA-MTX were measured using a spectrofluorimeter. A linear relationship between the dendrimer concentration and the fluorescence was observed at 10 to 1000 nM. The fluorescence of 100 nM solutions of G5-FITC, G5-FITC-FA and G5-FITC-FA-MTX were respectively 0.57, 0.23, and 0.11 spectrofluorimetric units. These differences in the fluorescence may be indicative of quenching due to the presence of FA and MTX on the dendrimer.

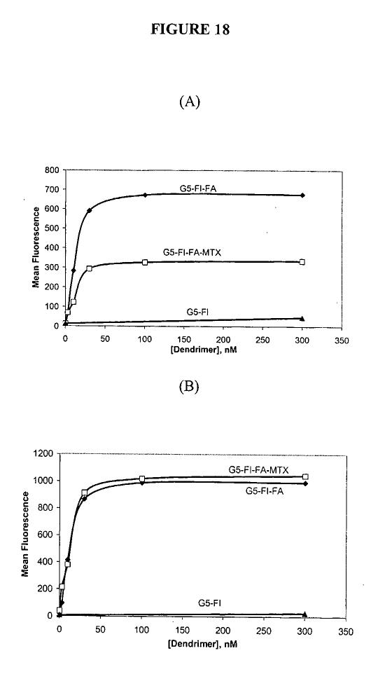

The cellular uptake of the dendrimers was measured in KB cells which express a high cell surface FA receptor (FAR). The FA-conjugated dendrimers bound to the cells in a dose-dependent fashion, with 50% binding at 10-15 nM for both the G5-FITC-FA and G5-FITC-FA-MTX, while the control dendrimer G5-FITC was not detected in the KB cells (See, e.g., FIG. 18A). Identical binding curves were obtained for the G5-FITC-FA and G5-FITC-FA-MTX when the fluorescence obtained was normalized for the quenching observed in the standard solutions of the dendrimers (See e.g., FIG. 18B). Analysis of the kinetics of the binding of the G5-FITC-FA-MTX (100 nM) showed that maximal binding was achieved within 30 minutes which is similar to reports for the binding of free folate.

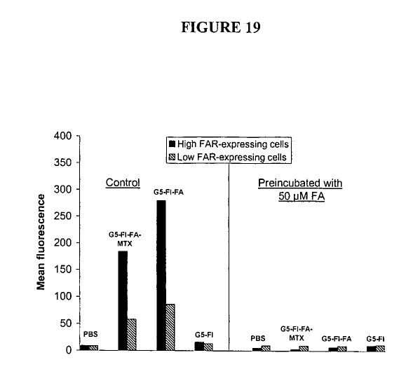

The effect of free FA on the uptake of the dendrimers was tested in KB cells that express both high and low FAR. The binding of the conjugates to the low FAR-expressing KB cells was 30% of that of the high FAR-expressing cells for both the G5-FITC-FA and G5-FITC-FA-MTX (See, for e.g., FIG. 19, left panel). 50 μM FA completely blocked the uptake of either targeted dendrimers (30 nM) in both the low- and high-FAR expressing cells (See, for e.g., FIG. 19, right panel). The binding and internalization of the dendrimers to KB cells was assessed by confocal microscopy. KB cells were incubated with 250 nM of the indicated dendrimers for 24 hours and confocal images were taken. Conjugates containing the targeting molecule FA internalized into KB cells within 24 h (See, e.g., FIG. 20). As compared to the cells treated with the control conjugates, the cells exposed to G5-FITC-FA-MTX were less adherent and rounded up, indicating cytotoxicity induced by the drug-conjugate.

### Example 10

### Functional Group Conjugated Dendrimers Inhibit Cell Growth

Because the binding of the conjugate to KB cells reaches maximal uptake within 1 h (Quintana et al, 2002. Pharmaceutical Research 19: 1310-1316.), the effect of the G5-FI-FA-MTX on cell growth was initially tested by pre-incubation of cells with the conjugate for 1 h, followed by incubation in a drug-free medium for 5 d. Under such conditions, the conjugate failed to show any growth-inhibitory effect in KB cells. When the cells were pre-incubated with dendrimers for 4 h, there was a modest decrease of about 10% in cell growth as determined by XTT assay. The cytotoxicity measurements were therefore done by incubation with the dendrimer for a minimum of 24 h, a pre-incubation time period shown to induce significant cytotoxicity.

Time course and dose dependent inhibition of cell growth. Previous studies have shown that MTX-induced cytotoxicity is detectable in vitro only if the medium is completely deprived of FA (See, e.g., Sobrero & Bertino, Int. J. Cell Cloning 4, 51 (1986)). The effect of the trifunctional dendrimers on cell growth was tested in cells incubated in a FA-deficient medium. Cells were treated with 300 nM conjugates (equivalent of 1500 nM MTX) or 1500 nM free MTX for 1-4 days, and cell proliferation was determined by estimation of cellular protein content. Cells were treated for 2 days with different concentrations of the conjugates or free MTX (the conjugate concentration is given as MTX equivalents, with 5 MTX per dendrimer molecule). KB cells which express high and low FAR were incubated with 30 nM of the dendrimers for 1 hr at 37° C., rinsed, and the fluorescence of cells was determined by flow cytometric analysis (See. e.g., FIG. 21, left panel). Pre-incubation with 50 μM free FA for 30 min totally prevents cellular binding and uptake of the polymer conjugates (See. e.g., FIG. 21, left panel).

The inhibition of cell growth induced by the conjugates was also tested by XTT assay which is based on the conversion of XTT to formazan by the active mitochondria of live cells (See, e.g., Roehm et al, J Immunol Methods 142, 257 (1991)). The G5-FITC or G5-FITC-FA were not growth-inhibitory for the cells at 1, 2 or 3 days, whereas the G5-FITC-FA-MTX and free MTX showed time-dependent cytotoxicity (See e.g., FIG. 22). Hence, the G5-FITC-FA-MTX and free MTX inhibited cell growth in a time- and dose-dependent fashion, whereas the control dendrimers failed to inhibit the cell growth (See, for e.g., FIGS. 21 and 22).

### Example 11

### Folic Acid Rescues Cells from Methotrexate Induce Cytotoxicity

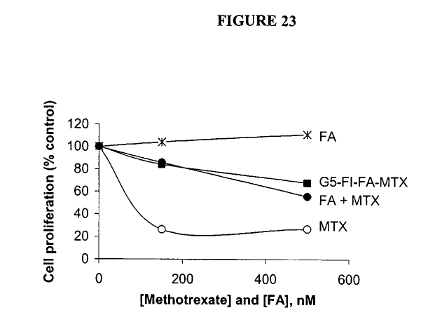

As growth inhibition induced by free MTX was higher than with the equimolar concentrations of MTX in the G5-FITC-FA-MTX below 1 μM (See, e.g., FIG. 21), it was tested whether the FA moiety in the G5-FITC-FA-MTX may be rescuing the cells from MTX-induced cytotoxicity. As the G5-FITC-FA-MTX preparation contained equimolar concentrations of MTX and FA, the effect of similar concentrations of free MTX and free FA on the inhibition of cell growth was determined. At equimolar concentrations of free FA and MTX, the FA reversed the inhibition of cell growth induced by MTX (See, e.g., FIG. 23). KB cells were treated with 150 or 500 nM MTX in the presence or absence of equimolar concentrations of free FA for 24 h. Cells were also treated with 30 and 100 nM G5-FI-FA-MTX (equivalent to 150 and 500 nM MTX) in parallel. The cells were rinsed to remove the drugs and incubated with fresh medium for an additional 6 d, and total cell protein was determined. The presence of 150 nM FA almost completely reversed the growth-arrest caused by 150 nM MTX. Moreover, the cytotoxicity induced by G5-FITC-FA-MTX (See, e.g., FIG. 23, filled square symbols) and equimolar combinations of PA and MTX (See, e.g., FIG. 23, filled circle symbols) was similar.

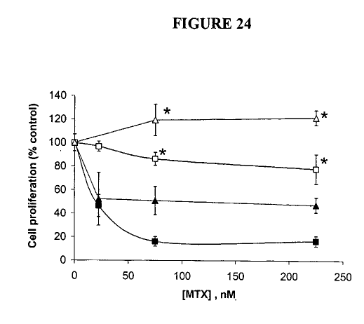

As free FA blocks the uptake of the dendrimers as well as rescues cells from MTX-induced cytotoxicity, the effect of pre-incubation of cells with excess FA on the anti-proliferative effect of G5-FITC-FA-MTX was tested. KB cells were exposed to different concentrations of the conjugate or free MTX for 24 h in the absence or presence of 50 μM FA. The incubation medium was removed, the cells were rinsed and incubated with fresh medium for 5 additional days in the absence of the drugs, and the XTT assay was performed (See, e.g., FIG. 24, □, ▪, represents cells treated with MTX; Δ, ▴, represents cells treated with G5-FITC-FA-MTX). Excess free FA not only blocked growth inhibition, but also increased cell growth 20% above that of the control cells (See, e.g., FIG. 24).

### Example 12

### Stability of Dendrimers

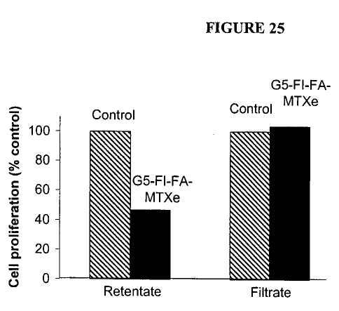

The stability of the dendrimer was tested in cell culture medium to check if MTX was released from the dendrimer prior to its entry into the cells. The G5-FITC-FA-MTX was incubated with cell culture medium for 1, 2, 4 and 24 h, and the incubation medium was filtered using a 10,000-MW cutoff ultrafiltration device. The effect of the retentate and the filtrate on the growth of the KB cells was tested. G5-FITC-FA-MTX was incubated with medium at 2 μM concentration for 24 h. The incubation medium was filtered through a Centricon 10K-MW cutoff filter. The retentate (adjusted to pre-filtration volume) and the filtrate were incubated with KB cells (at 200 nM conjugate, as determined from the concentration of the pre-filtration sample) for 2 days and the XTT assay was performed. Similar results were obtained for the retentate and filtrate obtained from the medium that had been pre-incubated with the dendrimers for 1, 2, and 4 hours. During the 24 h incubation time periods, the retentate was cytotoxic, whereas the filtrate failed to show any cytotoxicity (See, e.g., FIG. 25), indicating the lack of release of the free MTX from the conjugates. There was a slow release of the MTX after 24 h, reaching a maximum of 40-50% release in 1 week.

The anti-proliferative effect of the MTX-conjugates was compared to conjugates that lacked either the FA or the FITC molecule. KB cells were incubated with 30 nM of the conjugates (=150 nM effective MTX concentration) for 24 h and the incubation medium was removed. The cells were rinsed and incubated for an additional 5 d in fresh medium in the absence of the drugs, and the XTT assay was performed. The MTX-conjugated dendrimer that lacked FA failed to induce cytotoxicity, whereas the targeted dendrimer in the absence or presence of the dye molecule FITC induced cytotoxicity (See, e.g., FIG. 26).

### Example 13

### Use of Dendrimers to Target Tumors In Vivo

Compositions (e.g., multifunctional dendrimers) and methods of the present invention were used to determine therapeutic response in an animal model of cancer (e.g., human epithelial cancer).

Materials and reagents. All reagents were obtained from commercial sources. Folic acid, penicillin/streptomycin, fetal bovine serum, collagenase type IV, TX100, bis-benzimide, FITC, methotrexate, hydrogen peroxide, acetic anhydride, ethylenediamine, methanol, dimethylformamide, and DMSO were purchased from Sigma-Aldrich (St. Louis, Mo.). Trypsin-EDTA, Dulbecco's PBS, and RPMI 1640 (with or without folic acid) were from Invitrogen (Gaithersburg, Md.). “Solvable” solution and hionic fluor were from Packard Bioscience (Downers Grove, Ill.). OCT embedding medium was from Electron Microscopy Sciences (Fort Washington, Pa.), 2-methyl butane from Fisher Scientific (Pittsburgh, Pa.), and 6-carboxytetramethylrhodamine (6-TAMRA) and Prolong were from Molecular Probes, Inc. (Eugene, Oreg.). Tritium-labeled acetic anhydride (CH₃CO)₂O [³H] (100 mCi, 3.7 GBq) was purchased from ICN Biomedicals (Irvine, Calif.). Methotrexate for injection was from Bedford Laboratories (Bedford, Ohio). Folic acid was solubilized in saline, adjusted to pH 7.0 with 1 N NaOH, and filter sterilized for injections.

Synthesis and characterization of PAMAM dendrimer conjugates. A G5 PAMAM dendrimer was synthesized and purified from low molar mass contaminants as well as higher molar mass dimers or oligomers (See, e.g., Majoros et al., Macromolecules 36, 5529 (2003)). The number average molar mass of the dendrimer was determined to be 26,530 g/mol by size exclusion chromatography using multiangle laser light scattering, UV, and refractive index detectors. The average number of surface primary amine groups in the dendrimer was determined to be 110 using potentiometric titration along with the molar mass. The polydispersity index, defined as the ratio of weight average molar mass and number average molar mass for an ideal monodisperse sample, equals 1.0. The polydispersity index of G5 dendrimer was calculated to be 1.032, indicating very narrow distribution around the mean value and confirming the high purity of the G5 dendrimer. The surface amines of G5 PAMAM dendrimers were acetylated with acetic anhydride to reduce nonspecific binding of the dendrimer. The ratio between the acetic anhydride and the dendrimer was selected to achieve different acetylation levels from 50 to 80 and 100 primary amines. After purification, the acetylated dendrimer was conjugated to an imaging agent (e.g., FITC or 6-TAMRA) for detection and imaging. The imaging-conjugated (e.g., dye-conjugated) dendrimer was then allowed to react with an activated ester of a targeting agent (e.g., folic acid), and the purified product of this reaction was analyzed by ¹H nuclear magnetic resonance (NMR) to determine the number of conjugated targeting agents (e.g., folic acid molecules). Subsequently, a therapeutic agent (e.g., methotrexate) was conjugated via an ester bond (See, e.g., Quintana et al, Pharm Res 19, 1310 (2002)).

Radiolabeled compounds were synthesized from G5-(Ac)₅₀-(FA)₆ or G5-(Ac)₅₀ using tritiated acetic anhydride (Ac-3H) (See, e.g., Malik et al., J Control Release 65, 133 (2000); Nigavekar et al., Pharm Res 21, 476 (2004); Wilbur et al., Bioconjug Chem 9, 813 (1998)). The tritiated conjugates, G5-³H-FA and G5-³H, were fully acetylated. The specific activity of the G5-NHCOC-³H and G5-FA-NHCOC-³H conjugates were 10.27 and 38.63 mCi/g, respectively. The residual free tritium was <0.3% of the total activity.

The quality of the PAMAM dendrimer conjugates was tested using PAGE, ¹H NMR, ¹³C NMR, and mass spectroscopy. Capillary electrophoresis was used to confirm the purity and homogeneity of the final products.

The folic acid-targeted conjugates specifically contain the following molecules: G5-(Ac)₈₂-(FITC)₅-(FA)₅, G5-(Ac)₈₂-(6-TAMRA)₃-(FA)₄, G5-(Ac)₈₂-(FITC)₅-(FA)₅-MTX₅, and G5-(Ac)₅₀-(Ac-3H)₅₄-(FA)₆, which were identified with the acronyms G5-FI-FA, G5-6T-FA, G5-FI-FA-MTX, and G5-3H-FA, respectively. The nontargeted controls contained the following molecules: G5-(Ac)₈₂-(FITC)₅, G5-(Ac)₈₂-(6-TAMRA)₃, G5-(Ac)₈₂-(FITC)₅-MTX₅, and G5-(Ac)₅₀-(Ac-3H)₅₄, which were identified with the acronyms G5-FI, G5-6T, G5-FI-MTX, and G5-3H, respectively.

Recipient animal and tumor model. Immunodeficient, 6- to 8-weekold athymic nude female mice [Sim:(NCr) nu/nu fisol] were purchased from Simonsen Laboratories, Inc. (Gilroy, Calif.). Five- to 6-week-old Fox Chase severe combined immunodeficient (SCID; CB-17/lcrCrl-scidBR) female mice were purchased from the Charles River Laboratories (Wilmington, Mass.) and housed in a specific pathogen-free animal facility at the University of Michigan Medical Center in accordance with the regulations of the University's Committee on the Use and Care of Animals as well as with federal guidelines, including the Principles of Laboratory Animal Care. Animals were fed ad libitum with Laboratory Autoclavable Rodent Diet 5010 (PMI Nutrition International, St. Louis, Mo.). Three weeks before tumor cell injection, the food was changed to a folate-deficient diet (TestDiet, Richmond, Ind.). For urine and feces collection, animals were housed in metabolic rodent cages (Nalgene, Rochester, N.Y.).

Tumor cell line. The KB human cell line, which overexpresses the folate receptor (See, e.g., Turek et al, J Cell Sci 106, 423 (1993)), was purchased from the American Type Tissue Collection (Manassas, Va.) and maintained in vitro at 37° C., 5% CO₂ in folate-deficient RPMI 1640 supplemented with penicillin (100 units/mL), streptomycin (100 μg/mL), and 10% heat-inactivated fetal bovine serum. Before injection in the mice, the cells were harvested with trypsin-EDTA solution, washed, and resuspended in PBS. The cell suspension (5×10⁶ cells in 0.2 mL) was injected s.c. into one flank of each mouse using a 30-gauge needle. In the biodistribution studies, the tumors were allowed to grow for 2 weeks until reaching ˜0.9 cm³ in volume. The formula chosen to compute tumor volume was for a standard volume of an ellipsoid, where V= 4/3π (½ length×½ width×½ depth). With an assumption that width equals depth and k equals 3, the formula used was V=½×length×width². Targeted drug delivery using conjugate injections was started on the fourth day after implantation of the KB cells.

Biodistribution and excretion of tritiated dendrimer. Animals were injected via lateral tail vein with 0.5 mL PBS solution containing 174 μg G5-NHCOC-³H (1.8 ACi) or 200 μg G5-FA-NHCOC-3H (7.7 CCi). Both tritiumlabeled conjugates were delivered at equimolar concentrations of the modified dendrimer. At 5 minutes, 2 hours, 1 day, 4 days, and 7 days postinjection, the animals were euthanized and samples of tumor, heart, lung, liver, spleen, pancreas, kidney, and brain were taken. A third group of mice received a bolus of 80 μg free folic acid 5 minutes before injection with 200 μg G5-³H-FA. This 181 nmol concentration of free folic acid yields ˜150 μmol/L concentration in the blood compared with radiolabeled targeted dendrimer (G5-³H-FA), which yields ˜5 μmol/L concentration in the blood and is based on the 1.2 mL blood volume of a 20 g mouse. The mice were euthanized at 5 minutes, 1 day, and 4 days following injection, and tissues were harvested as above. Blood was collected at each time point via cardiac puncture. Each group included three to five mice. Urine and feces samples were collected at 2, 4, 8 and 12 hours and 1, 2, 3, and 4 days.

Radioactive tissue samples were prepared as described in Nigavekar et al, Pharm Res 21, 476 (2004). The tritium content was measured in a liquid scintillation counter (LS 6500, Beckman Coulter, Fullerton, Calif.). The values of measured radioactivity were adjusted for the counting efficiency of the instrument and used to derive radioactivity (1 μCi=2.22×10⁶ dpm) per sample. These values were then normalized by tissue weight and the specific radioactivity of the conjugates was reported as a percentage of the injected dosage (% ID/g). The excreted radioactivity (dendrimer) via urine and feces was reported as a percentage of the injected dosage (% ID).

Biodistribution of fluorescent dendrimer conjugates. Mice were injected via lateral tail vein with 0.5 mL saline solution containing 0.2 mg G5-6T or G5-6T-FA conjugates. At 15 hours and up to 4 days postinjection, the animals were euthanized and samples of tumor were taken and immediately frozen for sectioning and imaging. Flow cytometry analysis was done with single-cell suspension isolated from tumor. Tumor was crushed, cell suspension filtered through 70 μm nylon mesh (Becton Dickinson, Franklin Lakes, N.J.), and washed with in PBS. Samples were analyzed using an EPICS XL flow cytometer (Coulter, Miami, Fla.). As determined by prior propidium iodine staining, only live cells were gated for analysis. Data were reported as the mean channel fluorescence of the cell population.

For confocal microscope imaging, tissue was dissected, embedded in OCT, and frozen in 2-methyl-butane in a dry ice bath. Sections (15 μm) were cut on a cryostat, thaw mounted onto slides, and stored at −80° C. until stained. After staining, the slides were fixed in 4% paraformaldehyde, rinsed in phosphate buffer (0.1 mol/L; pH 7.2), and mounted in Prolong. The images were acquired using a Zeiss 510 metalaser scanning confocal microscope equipped with a ×40 Plan-Apo 1.2 numerical aperture (water immersion) objective with a correction collar. The confocal image was recorded as 512×512×48 pixels with a scale of 0.45×0.45×0.37 μm per pixel. Each image cube was optically cut into 48 sections, and the sections that cut through the nucleus and cytoplasm were presented.

Delivery of targeted nanoparticle therapeutic. Twice weekly, SCID mice with s.c. KB xenografts, starting on day 4 after tumor implantation, received via the tail vein an injection of either targeted or nontargeted conjugate containing methotrexate, a conjugate without methotrexate, free methotrexate, or saline as a control. The compounds were delivered in a 0.2 mL volume of saline per 20 g of mouse. The single dose of methotrexate delivered each time equaled 0.33 mg/kg. The higher doses of 1.67 and 3.33 mg/kg free methotrexate were also tested. The conjugates were delivered at equimolar concentration of methotrexate calculated based on the number of methotrexate molecules present in a nanoparticle. The conjugate without methotrexate was delivered at equimolar concentration of dendrimer. In the initial trial, six groups of mice with five mice in each group received up to 15 injections. In the follow-up trial, mice received up to 28 injections dependent on their survival. The body weights of the mice were monitored throughout the experiment as an indication of adverse effects of the drug. Histopathology of multiple organs was done at the termination of each trial and each time mouse had to be euthanized due to toxic effects or tumor burden. Tissues from lung, heart, liver, pancreas, spleen, kidney, and tumor were analyzed. Additionally, cells were isolated from tumors, stained with targeted fluorescein-labeled conjugate, and tested for the presence of folic acid receptors using flow cytometer.

Statistical methods. Means, SD, and SE of the data were calculated. Differences between the experimental groups and the control groups were tested using Student's-Newman-Keuls' test and Ps<0.05 were considered significant.

Biodistribution of tritiated dendrimers. The biodistribution and elimination of tritiated G5-³H-FA was first examined to test its ability to target the folate receptor-positive human KB tumor xenografts established in immunodeficient nude mice. The mice were maintained on a folate-deficient diet for the duration of the experiment to minimize the circulating levels of folic acid (See, e.g., Mathias et al., J Nucl Med 29, 1579 (1998)). The free folic acid level achieved in the serum of the mice before the experiment approximated human serum levels (See, Belz et al., Anal Biochem 265, 157 (1998); Nelson et al., Anal Biochem 325, 41 (2004)). Mice were evaluated at various time points (5 minutes to 7 days) following i.v. administration of the conjugates. Two groups of mice received either control nontargeted tritiated G5-³H dendrimer or targeted tritiated G5-³H-FA conjugate (FIGS. 27A and B). The conjugates were cleared rapidly from the blood via the kidneys during the first day postinjection, with the G5-³H decreasing from 23.4% D/g tissue at 5 minutes to 1.8% ID/g at 24 hours (FIG. 27A). The blood concentration of G5-³H-FA decreased from 29.1% ID/g at 5 minutes to 0.2% ID/g at 24 hours (FIG. 27B). In several organs, such as the lung, the tissue distribution showed a trend similar to blood concentrations with G5-³H decreasing from 9.7% ID/g at 5 minutes to 1.6% ID/g at 24 hours and G5-³H-FA decreasing from 9.6% ID/g at 5 minutes to 1.7% ID/g at 24 hours. Due to the high vascularity of the lung, conjugate levels measured at early time points likely reflect blood concentrations. Similar patterns of clearance were observed for the heart, pancreas, and spleen. These organs are known not to express folate receptor and do not show significant differences between the nontargeted and the targeted dendrimers. The concentrations of both G5-³H and G5-³H-FA in the brain were low at all time points, suggesting that the polymer conjugates did not cross the blood-brain barrier (FIGS. 27A and B). Although the kidney is the major clearance organ for these dendrimers, it is also known to express high levels of the folate receptor on its tubules. The level of nontargeted G5-³H in the kidney decreased rapidly and was maintained at a moderate level over the next several days (FIG. 27A). In contrast, the level of G5-³H-FA increased slightly over the first 24 hours most likely due to folate receptor present on the kidney tubules. This was followed by a decrease over the next several days as the compound was cleared through the kidney (FIG. 27B).

Both G5-³H and G5-³H-FA were rapidly excreted, primarily through the kidney, within 24 hours following injection. Incremental excretion of both compounds appeared entirely consistent with kidney retention of the conjugates (FIGS. 27A and B). Although both targeted and nontargeted conjugates also appeared in feces, it was in very low amounts. Whether any material was actually excreted in the feces was difficult to determine due to minor urine contamination of the feces. The cumulative clearance of the targeted G5-³H-FA over the first 4 days was lower than that of G5-³H, which may reflect retention of G5-³H-FA within tissues expressing folate receptors. The liver and KB tumor cells are known to express high levels of folate receptor. In these tissues, the concentrations of nontargeted G5-³H decreased rapidly with clearance of the dendrimer from the blood; the concentrations were maintained at a low level over the remaining days that the tissues were studied (FIG. 27A). In contrast, in both the liver and tumor, the targeted G5-³H-FA content increases over the first 4 days (FIG. 27B). This occurs during a time when blood levels of radioactive conjugate are low, suggesting specific uptake against a concentration gradient of dendrimer in these tissues, as opposed to the simple trapping of dendrimer through the vasculature.

The specificity of targeted drug delivery was further addressed in a group of mice receiving 181 nmol free folic acid before injection with G5-³H-FA (FIG. 27C). At 4 days after injection, significant attenuation in radioactivity related to the blocking of folate receptor with free folic acid was observed in tumor tissue that does not have the ability to excrete the dendrimer (FIG. 27C). This suggests that the difference in tumor concentrations between the targeted and the nontargeted polymer conjugates is due to the specific uptake of these molecules through the folate receptor overexpressed in the tumor. Distribution in all other tissues was not significantly altered by the delivery of free folic acid before the injection of the targeted conjugate.

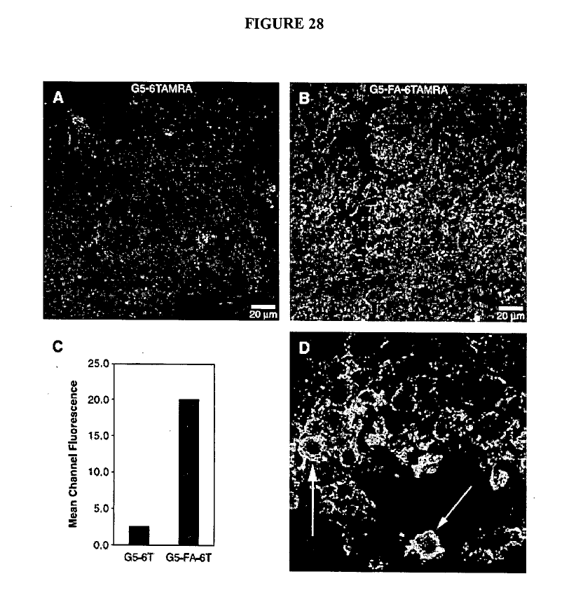

Targeting and internalization of fluorescent dendrimer conjugate. To further confirm and localize the dendrimer nanoparticles within tumor tissue, dendrimers conjugated with 6-TAMRA were employed. Confocal microscopy images were obtained of tumor samples at 15 hours following i.v. injection of the targeted G5-6T-FA and the nontargeted G5-6T conjugates (FIG. 28). The tumor tissue showed a significant number of fluorescent cells with targeted dye-conjugated dendrimer G5-6T-FA (FIG. 28B) compared with those with nontargeted dendrimer (FIG. 28A). Flow cytometry analysis of a single-cell suspension isolated from the same tumors showed higher mean channel fluorescence for tumor cells from mice receiving G5-6T-FA (FIG. 28C).

Confocal microscopy also showed that the conjugate is present in the tumors, attached to and internalized by many of the tumor cells (FIG. 28D). The optical overlapping sections were taken of the tissue slides from apical through medial to basal section. The medial section of tumor cells presented herein show fluorescence throughout the cytosol from the 6T of the conjugate, with the cell and nucleus boundary clearly visible (FIG. 28D).

Toxicity of dendrimer conjugates. All mice were observed for the duration of the studies for signs of dehydration, inability to eat or drink, weakness, or change in activity level. No gross toxicity, either acutely or chronically up to 99 days, was observed regardless of whether the dendrimer conjugate contained methotrexate. The weight was monitored throughout the experiment and no loss of weight was observed; in fact, the animals gained weight. At each time point, a gross examination and histopathology of the liver, spleen, kidney, lung, and heart were done. No morphologic abnormalities were observed on the histopathology examination. No in vivo toxicity was noted in any animal group following the dendrimer injection.

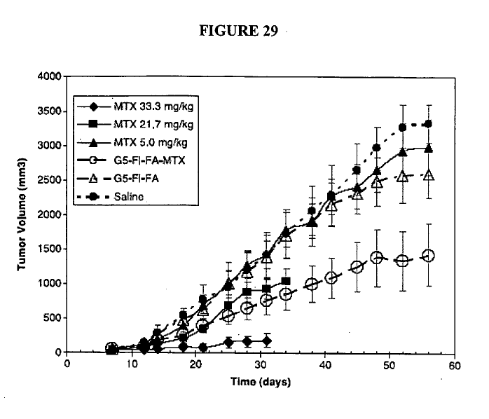

Targeted drug delivery to tumor cells through the folate receptor. The efficacy of different doses of conjugates was tested on SCID CB-17 mice bearing s.c. human KB xenografts and was compared with equivalent and higher doses of free methotrexate. Mice were maintained on the folic acid-deficient diet for 3 weeks before injection of the KB tumor cells to achieve circulating levels of folic acid that approach those in human serum and to prevent down-regulation of folate receptors on tumor xenografts (See, Mathias et al., J Nucl Med 29, 1579 (1998)). Six groups of SCID mice with five mice in each group were injected s.c. on one flank with 5×10⁶ KB cells in 0.2 mL PBS suspension. The highest total dose of G5-FI-FA-MTX therapeutic used equals 55.0 mg/kg and is equivalent to a 5.0 mg/kg total cumulative dose of free methotrexate (FIG. 29). The therapeutic dose of the conjugate was compared with three cumulative doses of free methotrexate equivalent to 33.3, 21.7, and 5.0 mg/kg accumulated in 10 to 15 injections based on mouse survival. Saline and the conjugate without methotrexate (G5-FI-FA) were used as controls.

The body weights of the mice were monitored throughout the experiment as an indication of adverse effects of the drug, and the changes of body weight showed acute and chronic toxicity in the highest and in the second highest cumulative doses of free methotrexate equal to 33.3 and 21.7 mg/kg, respectively. Although the two doses of free drug were affecting tumor growth, both became lethal by days 32 to 36 of the trial (FIG. 29). The remaining experimental groups had very uniform body weight fluctuations nonindicative of toxicity when compared with control groups with saline or conjugate without methotrexate. For the highest cumulative doses of free methotrexate used, histopathology analysis of the liver revealed advanced liver lesions, collections of inflammatory cells, and periportal inflammation. In contrast, neither the total accumulated dose of therapeutic conjugate equivalent to 5.0 mg/kg free methotrexate nor free methotrexate at the same dose were toxic (FIG. 29). Importantly, the therapeutic dose of conjugate that was equal to the lowest dose of free methotrexate used was as equally effective as the second highest dose of free methotrexate (21.7 mg/kg in 13 injections), whereas the free drug at this concentration had no effect on tumor growth (FIG. 29). The conjugate without methotrexate (G5-FI-FA) also had no therapeutic effect when compared with control injections of saline (FIG. 29). The liver slides from mice receiving the conjugate (G5-FI-FA-MTX) showed occasional periportal lymphocytes, indicating inflammation and single-cell necrosis that did not differ from that of control animals injected with saline.

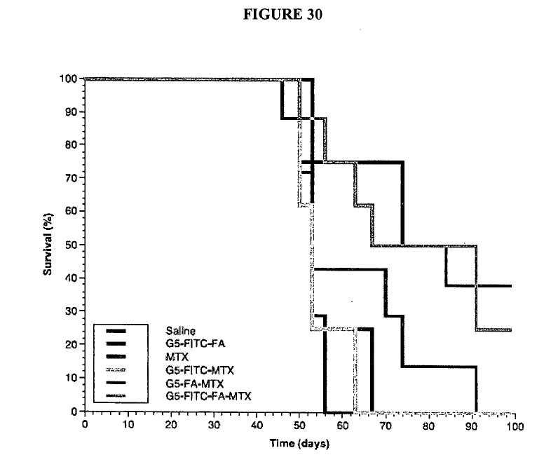

During a second 99-day trial, there was a statistically significant (P<0.05) slower growth of tumors that were treated with G5-FI-FA-MTX or G5-FA-MTX conjugate without FITC compared with those treated with nontargeted G5-FI-MTX conjugate, free methotrexate, or saline. The equivalent dose of methotrexate delivered with both targeted conjugates to the surviving mice was higher than the dose of free methotrexate because all of the mice receiving free methotrexate died by day 66 of the trial (FIG. 30). The survival of mice from groups receiving G5-FI-FA-MTX or G5-FA-MTX conjugate indicate that tumor growth based on the end-point volume of 4 cm3 can be delayed by at least 30 days (FIG. 30). This value indicates the antitumor effectiveness of the conjugate because it mimics clinical end-points and requires observation of the mice throughout the progression of the disease. Furthermore, a complete cure was obtained in one mouse treated with G5-FA-MTX conjugate at day 39 of the trial. The tumor in this mouse was not palpable for the next 20 days up to the 60th day of the trial. At the termination of the trial, there were three (of eight) survivors receiving G5-FA-MTX and two (of eight) survivors receiving G5-FI-FA-MTX. There were no mice surviving in the group receiving free methotrexate or in any other control group. Thus, in some embodiments, the present invention provides a composition comprising a dendrimer comprising a targeting agent, a therapeutic agent and an imaging agent. In preferred embodiments, the dendrimer is used for delivery, in a target specific manner, of a therapeutic agent (e.g., methotrexate) to tumor cells in vivo. The effective dose of conjugate was not toxic based on weight change and the histopathology examination that was done. At the termination of both trials, histopathology examination did not reveal signs of toxicity in the heart and myopathy did not develop. Acute tubular necrosis in the kidneys was not observed in these animals. Analysis of tumor slides showed viable tumors with mild necrosis in the control and saline-injected animals, whereas the therapeutic conjugate caused severe to significant necrosis in tumors compared with an equivalent dose of free methotrexate. At the termination of the trial, tumor cells were evaluated for possible up-regulation of folic acid receptor in tumor compared with KB cells due to a long-term folic acid-depleted diet of mice. Flow cytometry analysis of tumor cells after staining with targeted fluorescein-labeled conjugate revealed that cells remained folic acid receptor positive but at two to five times lower level compared with original KB cell line.

### Example 14

### PAMAM-Dendrimer—RGD4C Peptide Conjugate Synthesis

Drug targeting is critical for effective cancer chemotherapy. Targeted delivery enhances chemotherapeutic effect and spares normal tissues from the toxic side effects of these powerful drugs. Antiangiogenic therapy prevents neovascularization by inhibiting proliferation, migration and differentiation of endothelial cells (See, e.g., Los and Voest,

Semin. Oncol., 2001, 28, 93) The identification of molecular markers that can differentiate newly formed capillaries from their mature counterparts paved the way for targeted delivery of cytotoxic agents to the tumor vasculature (See, e.g., Baillie et al., Br. J. Cancer, 1995, 72, 257; Ruoslahti, Nat. Rev. Cancer, 2002, 2, 83; Arap et al., Science, 1998, 279, 377). The αᵥβ₃ integrin is one of the most specific of these unique markers.

The αᵥβ₃ integrin is found on the luminal surface of the endothelial cells only during angiogenesis. This marker can be recognized by targeting agents that are restricted to the vascular space during angiogenesis (See, e.g., Brooks et al., Science, 1994, 264, 569; Cleaver and Melton, Nat. Med,. 2003, 9, 661. High affinity αᵥβ₃ selective ligands, Arg-Gly-Asp (RGD) have been identified by phage display studies (Pasqualini et al., Nat. Biotech., 1997, 15, 542). The doubly cyclized peptide (RGD4C, containing two disulfide linkages via four cysteine residues) and a conformationally restrained RGD binds to αᵥβ₃ more avidly than peptides with a single disulfide bridge or linear peptides. There has been growing interest in the synthesis of polymer-RGD conjugates for gene delivery (See, e.g., Kunath et al., J. Gene. Med., 2003, 5, 588-599), tumor targeting (See, e.g., Mitra et al., J. Controlled Release, 2005, 102, 191) and imaging applications (See, e.g., Chen et al., J. Nucl. Med., 2004, 45, 1776).

In some embodiments, the present invention provides the synthesis of RGD4C conjugated to fluorescently labeled generation 5 dendrimer. Additionally the present invention provides the binding properties and cellular uptake of these conjugates.

Amine terminated dendrimers are reported to bind to the cells in a non-specific manner owing to positive charge on the surface. In order to improve targeting efficacy and reduce the non specific interactions, amine terminated G5 dendrimers were partially surface modified with acetic anhydride (75%×molar excess) in the presence of triethylamine as base (See e.g., Majoros et al., Macromolecules, 2003, 36, 5526. 4). The conjugate was purified by dialysis against PBS buffer initially and then against water. The use of 75 molar excess of acetic anhydride leaves some amine groups for further modification and prevents problems arising out of aggregation, intermolecular interaction and decreased solubility.

The degree of acetylation and purity of acetylated G5 dendrimer (G5-Ac) can be monitored using ¹H NMR spectroscopy. For detection of conjugates by flow cytometry or confocal microscopy a detectable probe (e.g., a fluorescent probe) can be used. For example, Alexa Fluor 488 (AF) can be used as a fluorescent label. The partially acetylated dendrimer was reacted with a 5 molar excess of Alexafluor-NHS ester as described in manufacturer's protocol to give fluorescently labeled conjugate (G5-Ac-AF). This conjugate was purified by gel filtration and subsequent dialysis. The number of dye molecules was estimated to be ˜3 per dendrimer by ¹H NMR and UV-vis spectroscopy as described in manufacturer's protocol (Molecular Probes):

The RGD peptide used in some embodiments of the present invention (RGD4C) has a conformationally restrained RGD sequence that binds specifically with high affinity to αᵥβ₃. The RGD binding site in the heterodimeric αᵥβ₃ integrin is located in a cleft between the two subunits. In order to keep the binding portion of the peptide exposed to the target site, an ε-Aca (acylhexanoic acid) spacer was used to conjugate the peptide to the dendrimer. A protonated NH₂ terminus of the RGD-4C peptide is not essential for biological activity therefore. Thus, in some embodiments, the NH₂ terminus is capped with an acetyl group (See, e.g., de Groot et al., Mol. Cancer. Therap., 2002, 1, 901).

An active ester of the peptide was prepared by using EDC in a DMF/DMSO solvent mixture in presence of HOBt, and then this was added dropwise to the aqueous solution of the G5-Ac-AF. The reaction times are 2 h and 3 days, respectively. The amidation occurs predominantly on the acylhexanoic acid linker carboxylate group (e.g., A model reaction with 1.1 eq. allyl amine in DMSO gave the mono amidated product in 67% purity (HPLC). ESI-MS m/z 1282 [M+H]⁺). The partially acetylated PAMAM dendrimer conjugated with AlexaFluor and RGD peptide, G5-Ac-AF-RGD was purified by membrane filtration and dialysis. The ¹H NMR of the conjugate shows overlapping signals in the aromatic region for both the AlexaFluor and phenyl ring of peptide apart from the expected aliphatic signals for the dendrimer. The number of peptides was calculated to be 2-3 peptides per dendrimer based on MALDI-TOF mass spectroscopy.

MALDI-TOF MS has been widely used technique for characterization of surface functionalization of heterogeneously functionalized dendrimers (See, e.g., Woller et al., J. Am. Chem. Soc., 2003, 125, 8820-8826).

Mass spectra were recorded on a Waters TOfspec-2E, run in delayed extraction mode, using the high mass PAD detector and calibrated with BSA in sinapinic acid. To determine the functionalization of the dendrimer with peptide (m/z 29650 [M+H]⁺) of the starting material was subtracted from the (m/z 32770 [M+H]⁺) of the product.

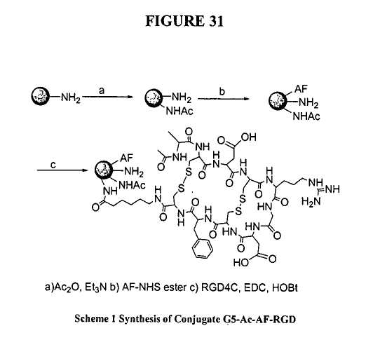

A schematic depicting the above described synthesis of G5-Ac-AF-RGD is shown in FIG. 31.

### Example 15

### In Vitro Targeting Efficacy of PAMAM-Dendrimer—RGD4C Peptide Conjugate

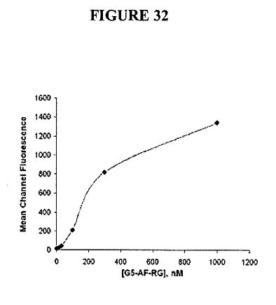

The cellular uptake of dendrimer-RGD4C conjugate was measured in Human umbilical vein endothelial cells (HUVEC) that express a high cell surface αᵥβ₃ receptor. In brief, HUVEC cells were cultured in RPMI medium supplemented with endothelial cell growth factor. The cells were treated with different concentrations of G5-Ac-AF-RGD conjugate and the uptake was monitored by flow cytometry. As shown in FIG. 32, flow cytometric analysis showed a dose-dependent and saturable binding to the HUVEC cells.

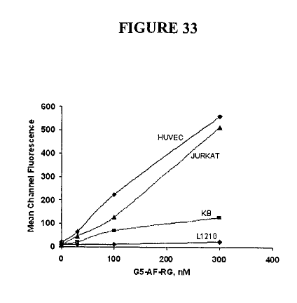

The binding of this conjugate to several different cell lines with varying levels of integrin receptor expression was also tested using flow cytometry (See, FIG. 33). The conjugate showed different binding affinities to various cell lines with HUVEC cells binding to the conjugate most effectively, followed by Jurkat cells. The human lymphocyte cell line Jurkat has previously been reported to have a large number of integrin receptors and was able to bind to RGD 4C peptide (See, e.g., Assa-Munt et al., Biochemistry, 2001, 40, 2373). The L1210 mouse lymphocyte line failed to bind the conjugate, whereas the KB cells showed only moderate binding.

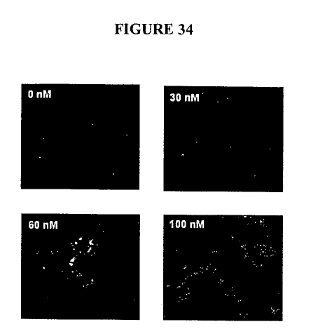

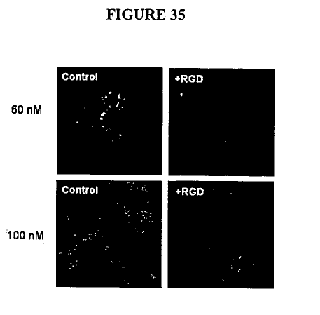

It is evident that the conjugate of the present invention shows variable specificities for cell lines having different levels of cell surface integrin receptor expression. The binding seen by flow cytometry was confirmed by confocal microscopic analysis. HUVEC cells treated with G5-AF-RGD4C (0, 30, 60, 100 nm) concentrations were washed and fixed with p-formaldehyde, the nuclei were counterstained with DAPI. It is evident from the appearance of fluorescence in confocal microscopic images in FIG. 34 that the uptake increases with the increasing concentration of the conjugate. The addition of free peptide inhibited the uptake of the conjugate by HUVEC cells to a significant level indicating receptor mediated uptake of the conjugate (See, FIG. 35).

In order to ascertain if polyvalent interaction shows stronger binding when compared to monovalent interaction, the binding affinity of G5-Ac-AF-RGD4C conjugate and RGD4C peptide were monitored on human integrin αᵥβ₃ purified protein (Chemicon International, Inc. Temecula, Calif.) using a BIAcore instrument (BIAcore AB, Uppsala, Sweden). The obtained data for both analytes was analyzed by global fitting to a bivalent binding model using the BIAevaluation 3.2 software (BIAcore AB). The equilibrium dissociation constants (K_{D}) were calculated from the ratio of the dissociation and association rate constants (k_{off}/kₒₙ). The binding of the free RGD4C peptide to the human integrin αᵥβ₃ was very rapid in reaching a maximum binding of 10 RU. On the contrary, the binding of the G5-Ac-AF-RGD4C conjugate was less rapid, reaching a maximum binding of approximately 1500 RU. Both analytes showed different off-rates. The free RGD4C peptide rapidly dissociated from the ligand during the washing time with running buffer. The nanodevice dissociation was approximately 522 times slower as compared to the free peptide. Thus, the present invention provides a multifunctional dendrimer wherein multiple peptide conjugation events on a single dendrimer exert a synergistic effect on binding efficacy.

Thus, the present invention provides PAMAM-dendrimer RGD4C peptide conjugates. In some embodiments, the dendrimer is taken up by cells expressing αᵥβ₃ receptors. Thus, in some preferred embodiments, the dendrimer conjugate is used to direct imaging agents and/or chemotherapeutics to angiogenic tumor vasculature.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising a dendrimer, said dendrimer comprising a partially acetylated generation 5 (G5) polyamideamine (PAMAM), polypropylamine (POPAM), or PAMAM-POPAM dendrimer, said dendrimer comprising two or more reactive sites for conjugation of a functional group.

2. The composition of claim 1, wherein said dendrimer comprises two or more functional groups, wherein said functional groups are selected from the group consisting of a therapeutic agent, a targeting agent, an imaging agent, and a biological monitoring agent.

3. The composition of claim 2, wherein at least one of said functional groups is conjugated to said dendrimers via an ester bond.

4. The composition of claim 2, wherein said therapeutic agent comprises methotrexate.

5. The composition of claim 2, wherein said targeting agent comprises folic acid.

6. The composition of claim 2, wherein said targeting agent comprises an RGD peptide.

7. The composition of claim 2, wherein said imaging agent comprises a fluorescing agent.

8. The composition of claim 7, wherein said fluorescing agent comprises fluorescein isothiocyanate.

9. The composition of claim 7, wherein said fluorescing agent comprises 6-TAMARA.

10. The composition of claim 4, wherein said methotrexate is conjugated to said dendrimer via an ester bond.

11. The composition of claim 1, wherein said dendrimer comprises between 2 and 20 reaction sites.

12. The composition of claim 2, wherein said dendrimer is conjugated to said functional groups.

13. The composition of claim 12, wherein said conjugation comprises covalent bonds, ionic bonds, metallic bonds, hydrogen bonds, Van der Waals bonds, ester bonds or amide bonds.

14. The composition of claim 2, wherein said therapeutic agent comprises a chemotherapeutic agent, an anti-oncogenic agent, an anti-vascularizing agent, a tumor suppressor agent, an anti-microbial agent, or an expression construct comprising a nucleic acid encoding a therapeutic protein.

15. The composition of claim 14, wherein said therapeutic agent is protected with a protecting group.

16. The composition of claim 15, wherein said protecting group is selected from the group consisting of photo-labile protecting group, a radio-labile protecting group, and an enzyme-labile protecting group.

17. The composition of claim 1, wherein said dendrimer comprises a protected core diamine.

18. The composition of claim 1, wherein said reactive sites comprise primary amine groups.

19. A composition comprising a dendrimer, said dendrimer comprising a partially acetylated G5 PAMAM, POPAM, or PAMAM-POPAM dendrimer, said dendrimer further comprising one or more functional groups, said one or more functional groups selected from the group consisting of a therapeutic agent, a targeting agent, and an imaging agent.

20. The composition of claim 19, wherein said therapeutic agent comprises an anti-oncogenic agent.

21. The composition of claim 19, wherein said therapeutic agent comprises a chemotherapeutic agent.

22. The composition of claim 19, wherein said therapeutic agent comprises methotrexate.

23. The composition of claim 19, wherein said therapeutic agent comprises tritium.

24. The composition of claim 19, wherein said targeting agent comprises folic acid.

25. The composition of claim 19, wherein said targeting agent comprises an RGD peptide.

26. The composition of claim 19, wherein said imaging agent comprises a fluorescing agent.

27. The composition of claim 26, wherein said fluorescing agent comprises fluorescein isothiocyanate.

28. The composition of claim 26, wherein said fluorescing agent comprises 6-TAMARA.

29. The composition of claim 22, wherein said methotrexate is conjugated to said dendrimer via an ester bond.

30. The composition of claim 19, wherein said therapeutic agent is selected from the group consisting of a chemotherapeutic agent, an anti-oncogenic agent, an anti-vascularizing agent, a tumor suppressor agent, an anti-microbial agent, and an expression construct comprising a nucleic acid encoding a therapeutic protein.

31. The composition of claim 19, wherein said therapeutic agent is protected with a protecting group.

32. The composition of claim 31, wherein said protecting group is selected from the group consisting of a photo-labile protecting group, a radio-labile protecting group, and an enzyme-labile protecting group.

33. The composition of claim 21, wherein said chemotherapeutic agent is selected from the group consisting of platinum complex, verapamil, podophylltoxin, carboplatin, procarbazine, mechloroethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, bleomycin, etoposide, tamoxifen, paclitaxel, taxol, transplatinum, 5-fluorouracil, vincristin, vinblastin, and methotrexate.

34. The composition of claim 20, wherein said anti-oncogenic agent comprises an antisense nucleic acid.

35. The composition of claim 34, wherein said antisense nucleic acid comprises a sequence complementary to an RNA of an oncogene.

36. The composition of claim 35, wherein said oncogene is selected from the group consisting of abl, Bcl-2, Bcl-xL, erb, fms, gsp, hst, jun, myc, neu, raf, ras, ret, src, and trk.

37. The composition of claim 30, wherein said nucleic acid encodes a protein selected from the group consisting of a tumor suppressor, a cytokine, a receptor, an inducer of apoptosis, and a differentiating agent.

38. The composition of claim 37, wherein said tumor suppressor is selected from the group consisting of BRCA1, BRCA2, C-CAM, p16, p21, p53, p73, Rb, and p27.

39. The composition of claim 37, wherein said cytokine is selected from the group consisting of GMCSF, IL-1, IL-2, IL-3, IL-4, IL-5, IL-6, IL-7, IL-8, IL-9, IL-10, IL-11, IL-12, IL-13, IL-14, IL-15, IFN-β, IFN-γ, and TNF.

40. The composition of claim 37, wherein said receptor is selected from the group consisting of CFTR, EGFR, estrogen receptor, IL-2 receptor, and VEGFR.

41. The composition of claim 37, wherein said inducer of apoptosis is selected from the group consisting of AdE1B, Bad, Bak, Bax, Bid, Bik, Bim, Harakid, and ICE-CED3 protease.

42. The composition of claim 19, wherein said therapeutic agent comprises a short half-life radioisotope.

43. The composition of claim 19, wherein said imaging agent comprises a radioactive label selected from the group consisting of ¹⁴C, ³⁶C1, ⁵⁷Co, ⁵⁸Co, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, Tc-99m, and ¹⁷⁵Yb.

44. The composition of claim 19, wherein said targeting agent is selected from the group consisting of an antibody, a receptor ligand, a hormone, a vitamin, and an antigen.

45. The composition of claim 44, wherein said antibody is specific for a disease specific antigen.

46. The composition of claim 45, wherein said disease specific antigen comprises a tumor specific antigen.

47. The composition of claim 44, wherein said receptor ligand is selected from the group consisting of a ligand for CFTR, a ligand for FGFR, a ligand for estrogen receptor, a ligand for FGR2, a ligand for folate receptor, a ligand for IL-2 receptor, a glycoprotein, a ligand for EGFR, and a ligand for VEGFR.

48. The composition of claim 44, wherein said receptor ligand is folic acid.

49. The composition of claim 44, wherein said receptor ligand is an RGD peptide.

50. A method of treating a disease comprising administering to a subject suffering from or susceptible to said disease a therapeutically effective amount of the composition of claim 19.

51. The method of claim 50, wherein said disease is a neoplastic disease.

52. The method of claim 51, wherein said neoplastic disease is selected from the group consisting of leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

53. A method of altering tumor growth in a subject, comprising:
a) providing a composition comprising a dendrimer, said dendrimer comprising a partially acetylated G5 PAMAM, POPAM, or PAMAM-POPAM dendrimer, said dendrimer further comprising one or more functional groups, said one or more functional groups selected from the group consisting of a therapeutic agent, a targeting agent, and an imaging agent; and
b) administering said composition to said subject under conditions such that said tumor growth is altered.

54. The method of claim 53, wherein said altering comprises inhibiting tumor growth in said subject.

55. The method of claim 53, wherein said altering comprises reducing the size of said tumor in said subject.

56. The method of claim 53, wherein said composition comprising a dendrimer is co-administered with a chemotherapeutic agent or anti-oncogenic agent.

57. The method of claim 53 wherein said altering tumor growth sensitizes said tumor to chemotherapeutic or anti-oncogenic treatment.

58. The method of claim 53, wherein said therapeutic agent comprises an anti-oncogenic agent.

59. The method of claim 53, wherein said therapeutic agent comprises a chemotherapeutic agent.

60. The method of claim 53, wherein said therapeutic agent comprises methotrexate.

61. The method of claim 53, wherein said therapeutic agent comprises tritium.

62. The method of claim 53, wherein said targeting agent comprises folic acid.

63. The method of claim 53, wherein said targeting agent comprises an RGD peptide.

64. The method of claim 53, wherein said imaging agent comprises a fluorescing agent.

65. The method of claim 53, wherein said fluorescing agent comprises fluorescein isothiocyanate.

66. The method of claim 53, wherein said fluorescing agent comprises 6-TAMARA.

67. The method of claim 53, wherein said methotrexate is conjugated to said dendrimer via an ester bond.

68. The method of claim 53, wherein said therapeutic agent is selected from the group consisting of a chemotherapeutic agent, an anti-oncogenic agent, an anti-vascularizing agent, a tumor suppressor agent, an anti-microbial agent, and an expression construct comprising a nucleic acid encoding a therapeutic protein.

69. The method of claim 53, wherein said therapeutic agent is protected with a protecting group.

70. The method of claim 69, wherein said protecting group is selected from the group consisting of a photo-labile protecting group, a radio-labile protecting group, and an enzyme-labile protecting group.

71. The method of claim 59, wherein said chemotherapeutic agent is selected from the group consisting of platinum complex, verapamil, podophylltoxin, carboplatin, procarbazine, mechloroethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, bleomycin, etoposide, tamoxifen, paclitaxel, taxol, transplatinum, 5-fluorouracil, vincristin, vinblastin, and methotrexate.

72. The method of claim 58, wherein said anti-oncogenic agent comprises an antisense nucleic acid.

73. The method of claim 72, wherein said antisense nucleic acid comprises a sequence complementary to an RNA of an oncogene.

74. The method of claim 73, wherein said oncogene is selected from the group consisting of abl, Bcl-2, Bcl-xL, erb, fins, gsp, hst, jun, myc, neu, raf, ras, ret, src, and trk.

75. The method of claim 68, wherein said nucleic acid encodes a protein selected from the group consisting of a tumor suppressor, a cytokine, a receptor, an inducer of apoptosis, and a differentiating agent.

76. The method of claim 75, wherein said tumor suppressor is selected from the group consisting of BRCA1, BRCA2, C-CAM, p16, p21, p53, p73, Rb, and p27.

77. The method of claim 75, wherein said cytokine is selected from the group consisting of GMCSF, IL-1, IL-2, IL-3, IL-4, IL-5, IL-6, IL-7, IL-8, IL-9, IL-10, IL-11, IL-12, IL-13, IL-14, IL-15, IFN-β, IFN-γ, and TNF.

78. The method of claim 75, wherein said receptor is selected from the group consisting of CFTR, EGFR, estrogen receptor, IL-2 receptor, and VEGFR.

79. The method of claim 75, wherein said inducer of apoptosis is selected from the group consisting of AdE1B, Bad, Bak, Bax, Bid, Bik, Bim, Harakid, and ICE-CED3 protease.

80. The method of claim 53, wherein said therapeutic agent comprises a short half-life radioisotope.

81. The method of claim 53, wherein said imaging agent comprises a radioactive label selected from the group consisting of ¹⁴C, ³⁶Cl, ⁵⁷Co, ⁵⁸Co, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, Tc-99m, and ¹⁷⁵Yb.

82. The method of claim 53, wherein said targeting agent is selected from the group consisting of an antibody, a receptor ligand, a hormone, a vitamin, and an antigen.

83. The method of claim 82, wherein said antibody is specific for a disease specific antigen.

84. The method of claim 83, wherein said disease specific antigen comprises a tumor specific antigen.

85. The method of claim 82, wherein said receptor ligand is selected from the group consisting of a ligand for CFTR, a ligand for FGFR, a ligand for estrogen receptor, a ligand for FGR2, a ligand for folate receptor, a ligand for IL-2 receptor, a glycoprotein, a ligand for EGFR, and a ligand for VEGFR.

86. The method of claim 82, wherein said receptor ligand is folic acid.

87. The method of claim 82, wherein said receptor ligand is an RGD peptide.

88. The method of claim 53, wherein said tumor is associated with a neoplastic disease.

89. The method of claim 88, wherein said neoplastic disease is selected from the group consisting of leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.


SEQUENCE LISTING (1 sequence)

SEQ ID NO: 1 - protein, 7 aa, Artificial Sequence
  Synthetic
YEVDGWK